(19) United States
(12) Patent Application Publication
(10) Pub. No.: US 2026/0234699 A1
(43) Pub. Date: Aug. 13, 2026

(54) TISSUE RESIDENT MEMORY CELL PROFILES, AND USES THEREOF IN INFLAMMATORY AND AUTOIMMUNE DISEASES

(71) Applicants: Simon Eschwiler, La Jolla, CA (US); Syed Hassan Arshad, Southampton, Hampshire (GB); Ramesh J. Kurukulaaratchy, Southampton, Hampshire (GB); La Jolla Institute for Immunology, La Jolla, CA (US); University of Southampton, Southampton, Hampshire (GB)

(72) Inventors: Simon Eschwiler, La Jolla, CA (US); Syed Hassan Arshad, Southampton, Hampshire (GB); Ramesh J. Kurukulaaratchy, Southampton, Hampshire (GB); Pandurangan Vijayanand, La Jolla, CA (US); Grégory Seumois, La Jolla, CA (US)

(21) Appl. No.: 18/701,170

(22) PCT Filed: Oct. 17, 2022
(86) PCT No.: PCT/US2022/046892

§ 371 Date: Apr. 12, 2024

Related U.S. Application Data
(60) Provisional application No. 63/256,496, filed on Oct. 15, 2021.

(30) Foreign Application Priority Data
Oct. 14, 2022 (WO) PCT/US2022/046798

(51) Int. Cl. C12Q 1/6809; A61K 38/00; A61K 45/06; A61P 37/00; C07K 16/00

(52) U.S. Cl. CPC C12Q 1/6809; A61K 38/00; A61K 45/06; A61P 37/00; C07K 16/00; C12Q 2600/106; C12Q 2600/112; C12Q 2600/118; C12Q 2600/158; G01N 2800/24; G01N 2800/52

(57) ABSTRACT

This disclosure provides methods of treating autoimmune disease or conditions, inflammatory disease, and/or aberrant immune responses, including asthma and fibrotic disease, in a subject by modulating a population of T-cells that exhibits higher or lower than baseline expression of the one or more genes disclosed herein. In other aspects, methods are provided to diagnose such disease and determine prognosis of such patients. Also provided are methods to identify markers associated with the isolated and/or purified cell populations that elicit a more positive prognosis.

### CROSS-REFERENCE TO RELATED APPLICATIONS

This application is a U.S. National Phase Application under 35 U.S.C. 371 of International Application No. PCT/US2022/046892, filed Oct. 17, 2022, which claims priority under 35 U.S.C. § 119 (e), and under the Paris Convention to U.S. Provisional Application No. 63/256,496, filed Oct. 15, 2021, and PCT/US2022/046798, filed Oct. 14, 2022, the contents of each of which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under grant number R01 HL114093 awarded by the National Institutes of Health/NIAID. The government has certain rights in the invention.

### BACKGROUND

Asthma is one of the most common chronic diseases affecting children and adults (1-4). The classical symptoms of asthma like wheeze, cough, and breathlessness, are triggered by inflammation and remodeling of the airways that results in airway hyperreactivity and obstruction (2, 5, 6). The airway inflammation in asthmatic patients is considered to be driven by type 2 cytokine-producing CD4⁺ T cells (T_{H}2) that play a central role in orchestrating recruitment and activation of innate immune cells such as eosinophils, basophils, and mast cells (2, 3, 5-7). Suppressing airway inflammation with corticosteroids remains the mainstay of treatment for most patients with asthma (8-10). However, many patients with severe forms of asthma fail to respond well to corticosteroids and suffer from persistent symptoms and recurrent life-threatening exacerbations (8). Moreover, the recently approved biological agents blocking type 2 cytokines or immunoglobulin (Ig) E are neither uniformly effective nor do they reverse disease pathogenesis in severe asthmatics (3, 11-13). These observations raise the possibility that other immune cells may contribute to airway inflammation and remodeling in severe asthmatics.

Here, to define the immune cell subsets and their properties associated with severe asthma and corticosteroid resistance, and by extension, autoimmune diseases, allergic diseases, and fibrotic diseases, the inventors performed single-cell RNA-seq assays from purified immune cells isolated from the airways of patients with severe and mild asthma. Disclosed herein, inter alia, are solutions to these and other problems in the art.

### SUMMARY OF THE DISCLOSURE

To address the above identified limitations in the art, this disclosure provides methods of treating autoimmune disease, inflammatory disease, and/or aberrant immune responses, including asthma, or eliciting an anti-inflammatory response in a subject in need thereof, the methods comprising, or consisting essentially of, or consisting of administering to the subject an agent to block the activity of a population of T-cells that exhibits higher or lower than baseline expression of one or more select genes. In alternative aspects, the disclosure provides methods of administering an agent capable of modulating the expression, activity, or proliferation of a population of T-cells that exhibits higher or lower than baseline expression of one or more genes. In one aspect, this method comprises, or consists essentially of, or yet further consists of administering to the subject an effective amount of an active agent that induces higher or lower than baseline expression of one or more genes or product thereof. In particular embodiments, the one or more gene is AMICA1 (also known as JAML). In alternative embodiments, the gene product is the Junction Adhesion Molecule Like protein (JAML). In certain embodiments, the agent is an antibody, a small molecule, a protein, a peptide, a ligand mimetic or a nucleic acid capable of modulating the activity or expression of JAML or JAML-expressing cells. In other embodiments, the agent is an antibody capable of inhibiting JAML or JAML-expressing cells.

For the disclosed methods, in one aspect, the one or more genes are set forth herein, or set forth on the accompanying Figures. In another aspect, the one or more genes are selected from or alternatively comprise, consist of, or consist essentially of CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1. In some aspects, the one or more genes comprise, consist of, or consist essentially of AMICA1.

Also provided herein are methods to reduce or inhibit an immune response and treat conditions requiring selective immunotherapy, comprising, or consisting essentially of, or yet further consisting of, contacting a target cell with the cells or compositions as described herein. The contacting can be performed in vitro, or alternatively in vivo, thereby reducing or inhibiting an immune response and to treat conditions requiring selective immunotherapy to a subject such as for example, a human patient.

In one aspect, this disclosure provides a method of treating asthma or an autoimmune or fibrotic disease in a subject comprising, consisting of, or consisting essentially of administering to the subject an effective amount of an agent to block the activity of a population of T-cells that exhibit higher than or lower than baseline expression of one or more genes selected from or alternatively comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1, thereby treating asthma or the autoimmune or fibrotic disease in the subject.

In one aspect, this disclosure provides a method of treating asthma or an autoimmune or fibrotic disease in a subject comprising, consisting of, or consisting essentially of administering to the subject an effective amount of an agent that induces higher than or lower than baseline expression of one or more genes selected from or alternatively comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in T-cells, thereby treating asthma or the autoimmune or fibrotic disease in the subject.

In one aspect, this disclosure provides a method of treating asthma or an autoimmune or fibrotic disease in a subject or sample comprising, consisting of, or consisting essentially of administering an effective amount of one or more of an agent that induces or inhibits in T-cells activity of one or more proteins encoded by genes selected from or alternatively comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 to the subject, thereby treating asthma or the autoimmune or fibrotic disease in the subject. In some aspects, the one or more proteins encoded comprise, consist of, or consist essentially of JAML. In certain embodiments, the agent is an antibody capable of inhibiting JAML.

In some aspects, the active agent comprises, consists of, or consists essentially of an antibody, a small molecule, a protein, a peptide, a ligand mimetic or a nucleic acid.

In some aspects of the methods disclosed herein, the immune cells comprise, consist of, or consist essentially of T cells. In other aspects, the T cells comprise, consist of, or consist essentially of CD4+ T-cells or CD8+ T-cells. In some aspects, the T cells comprise, consist of, or consist essentially of tissue resident memory (TRM) cells. In certain aspects, the T cells or TRM cells are cytotoxic T cells. In some aspects, the T cells are autologous to the subject being treated. In one aspect the T cells are mammalian T cells, e.g., human T cells and the species of the cells is identical to the species being treated.

As provided herein, the condition, disease, or disorder capable of being treated by the aspects disclosed herein is an autoimmune or asthma disease, disorder, or condition, a proinflammatory condition, and allergic disease, fibrotic disease, or an aberrant immune response. In certain embodiments, an autoimmune or asthma disease, disorder, or condition, a proinflammatory condition, and allergic disease, fibrotic disease, or an aberrant immune response comprises, consists of, or consists essentially of polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, nonalcoholic fatty liver disease, primary biliary cholangitis, autoimmune hepatitis, autoimmune kidney disease, chronic obstructive pulmonary disease (COPD), Castleman's disease, asthma (mild intermittent, mild persistent, moderate persistent, or severe persistent), allergic asthma (mild intermittent, mild persistent, moderate persistent, or severe persistent), allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenia purpura, pernicious anemia, sensorineural hearing loss, idiopathic bilateral progressive sensorineural hearing loss, autoimmune polyglandular syndrome type I or type II, immune infertility and immune-mediated infertility.

In other aspects, provided are one or more methods of diagnosing autoimmune disease or conditions, inflammatory disease, and/or aberrant immune responses, including asthma, identifying a subject likely to benefit from or respond to treatment, (including but not limited to immunotherapy (including anti-autoimmune disease or conditions, inflammatory disease, and/or aberrant immune responses, including asthma, or anti-asthma immunotherapy)), determining the effectiveness of treatment, and/or determining a prognosis of a subject having autoimmune disease or conditions, inflammatory disease, and/or aberrant immune responses, including asthma. The one or more methods comprise, or alternatively consist essentially of, or yet further consist of, detecting or measuring the population or amount of TRMs, or a sub-population of TRMs expressing high levels of one or more of the genes set forth herein, or set forth on the accompanying Figures, in the subject or in a sample isolated from the subject. In certain embodiments, a lower amount of TRMs or lower amount of the sub-population of TRMs expressing high levels of one or more of the genes set forth herein, or set forth on the accompanying Figures, in the subject or sample indicates that the subject is likely to benefit from or respond to treatment, that the treatment likely to be effective in the subject, or that the subject is likely to proceed have a positive clinical response. In certain embodiments, a higher amount of TRMs or higher amount of the sub-population of TRMs expressing high levels of one or more of the genes set forth herein, or set forth on the accompanying Figures, in the subject or sample indicates that the subject is less likely to benefit from or respond to treatment, that the treatment is likely not as effective in the subject as other therapies, or that the subject has a poor prognosis with available therapies.

In yet another aspect, this disclosure provides a method of diagnosing asthma or an autoimmune or fibrotic disease in a subject, comprising, consisting of, or consisting essentially of contacting a sample isolated from the subject with an agent that detects the presence of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in the sample isolated from the subject, wherein the presence of the one or more genes at higher or lower than baseline expression levels is a diagnostic indicator of asthma or the autoimmune or fibrotic disease or wherein the absence of the one or more genes at higher or lower than baseline expression levels is not diagnostic indicator of asthma or the autoimmune or fibrotic disease.

In one aspect, this disclosure provides a method of diagnosing asthma or an autoimmune or fibrotic disease in a subject comprising, consisting of, or consisting essentially of contacting tissue-resident memory cells (TRMs) isolated from the subject or a sample isolated from the subject, with an antibody or agent that recognizes and binds one or more proteins encoded by a gene comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 to determine the frequency of TRMs expressing these proteins, wherein a high frequency of TRMs expressing these proteins is diagnostic of asthma or the autoimmune or fibrotic disease.

In one aspect, this disclosure provides a method of determining the density of tissue-resident memory cells (TRMs) in a sample isolated from a subject comprising, consisting of, or consisting essentially of measuring expression of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in the sample, wherein higher or lower than baseline expression indicates higher density of TRMs in the sample thereof.

In one aspect, this disclosure provides a method of determining prognosis of a subject having an autoimmune or fibrotic disease comprising, consisting of, or consisting essentially of measuring the density of tissue-resident memory cells (TRM) in a sample isolated from the subject, wherein a low density of TRM indicates a more positive prognosis or wherein a high density or TRM indicates a more negative prognosis optionally wherein the more negative prognosis comprises a decreased probability in the reduction of symptoms of the autoimmune or fibrotic disease and a lack of low density TRM indicates a more positive prognosis, optionally increased probability in the reduction of symptoms of the autoimmune or fibrotic disease.

In one aspect, this disclosure provides a method of determining prognosis of a subject suffering from an autoimmune or fibrotic disease comprising, consisting of, or consisting essentially of contacting tissue-resident memory cells (TRMs) isolated from the subject with one or more of: an antibody or agent that recognizes and binds one or more proteins encoded by a gene comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1, wherein a low density of TRMs indicates a more positive prognosis or wherein a high density of TRMs indicates a more negative prognosis, optionally wherein the more negative prognosis comprises a decreased probability in the reduction of symptoms of the autoimmune or fibrotic disease, and wherein the more positive prognosis comprises an increased probability in the reduction of symptoms of the autoimmune or fibrotic disease.

In one aspect, this disclosure provides method of determining prognosis of a subject having an autoimmune or fibrotic disease comprising, consisting of, or consisting essentially of contacting tissue-resident memory cells (TRMs) isolated from the subject with an antibody or agent that recognizes and binds one or more proteins encoded by a genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs expressing these proteins, wherein a low density of TRMs indicates a more positive prognosis or wherein a high density of TRMs indicates a more negative prognosis, optionally wherein the more negative prognosis comprises, consists of, or consists essentially of a decreased probability in the reduction of symptoms of the autoimmune or fibrotic disease, and wherein the more positive prognosis comprises, consists of, or consists essentially of an increased probability in the reduction of symptoms of the autoimmune or fibrotic disease.

In one aspect, this disclosure provides a method of determining prognosis of a subject having an autoimmune or fibrotic disease comprising, consisting of, or consisting essentially of contacting tissue-resident memory cells (TRMs) isolated from the subject with an antibody or agent that recognizes and binds CD103 to determine the frequency of CD103+ TRMs or an antibody that recognizes and binds a protein encoded by a genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs expressing the protein, wherein a low density of TRMs indicates a more positive prognosis or wherein a high density of TRMs indicates a more negative prognosis, optionally wherein the more negative prognosis comprises, consists of, or consists essentially of a decreased probability in the reduction of symptoms of the autoimmune disease or fibrotic, and wherein the more positive prognosis comprises, consists of, or consists essentially of an increased probability in the reduction of symptoms of the autoimmune or fibrotic disease.

In one aspect, this disclosure provides a method of determining the responsiveness of a subject having asthma or an autoimmune or fibrotic disease to immunotherapy comprising, consisting of, or consisting essentially of contacting tissue-resident memory cells (TRMs) isolated from the subject with an antibody or agent that recognizes and binds one or more proteins encoded by a genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs in the subject, wherein a high frequency of TRMs indicates lack of responsiveness to immunotherapy.

In one aspect, this disclosure provides a method of determining the responsiveness of a subject having asthma or an autoimmune or fibrotic disease to immunotherapy comprising, consisting of, or consisting essentially of contacting tissue-resident memory cells (TRMs) isolated from the subject with an antibody or agent that recognizes and binds one or more proteins encoded by a genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 and, to determine the frequency of TRMs expressing these proteins, wherein a low frequency of TRMs expressing these proteins indicates responsiveness to immunotherapy.

In one aspect, this disclosure provides a method of determining prognosis of a subject having an autoimmune disease or fibrotic comprising, consisting of, or consisting essentially of measuring the density of CD103 or proteins encoded by one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in a sample isolated from the subject, wherein a low density of TRM indicates a more positive prognosis or wherein a high density or TRM indicates a more negative prognosis optionally wherein the more negative prognosis comprises, consists of, or consists essentially of a decreased probability in the reduction of symptoms of the autoimmune or fibrotic disease and a lack of low density TRM indicates a more positive prognosis, optionally increased probability in the reduction of symptoms of the autoimmune or fibrotic disease.

In one aspect, this disclosure provides a method of identifying a subject that will or is likely to respond to asthma therapy or an autoimmune or fibrotic disease therapy, comprising, consisting of, or consisting essentially of contacting a sample isolated from the subject with an agent that detects the presence of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 in the sample, wherein the presence of the one or more genes at higher or lower than baseline expression levels indicates that the subject is likely to respond to the asthma therapy or the autoimmune or fibrotic disease therapy.

In some aspects of this disclosure, baseline expression comprises, consists of, or consists essentially normalized mean gene expression. In yet another aspect, higher than baseline expression of the one or more genes is at least about a 2-fold increase in expression relative to baseline expression and/or lower than baseline expression of the one or more genes is at least about a 2-fold decrease in expression relative to baseline expression. In some aspects, the methods further comprise, consist of, or consist essentially of administering an asthma therapy or an autoimmune or fibrotic disease therapy to the subject. In some aspects the asthma therapy or an autoimmune or fibrotic disease therapy comprises, consists of, or consists essentially of one or more of hormonal therapy, immunotherapy, bronchodilators, corticosteroids, monoclonal antibodies, and/or administering to the subject an effective amount of an agent as disclosed herein. In some aspects, the samples are labeled with an agent, detectable label, or tag. In some aspects, the detectable label or tag comprises, consists of, or consists essentially of radioisotope, a metal, horseradish peroxidase, alkaline phosphatase, avidin or biotin. In some aspects, the agent comprises, consists of, or consists essentially of a polypeptide that binds to an expression product encoded by the gene, or a polynucleotide that hybridizes to a nucleic acid sequence encoding all or a portion of the gene. In some aspects, the polypeptide comprises, consist of, or consists essentially of an antibody, an antigen binding fragment thereof, or a receptor that binds to the gene. In some aspects the antibody comprises, consists of, or consists essentially of antibody is an IgG, IgA, IgM, IgE or IgD, or a subclass thereof. In some aspects, the IgG comprises, consists of an IgG1, IgG2, IgG3 or IgG4. In some aspects, the agent is contacted with the sample in conditions under which it can bind to the gene it targets. In some aspects, the methods provided herein comprise, consist of, or consist essentially of detection by immunohistochemistry (IHC), in-situ hybridization (ISH), ELISA, immunoprecipitation, immunofluorescence, chemiluminescence, radioactivity, X-ray, nucleic acid hybridization, protein-protein interaction, immunoprecipitation, flow cytometry, Western blotting, polymerase chain reaction, DNA transcription, Northern blotting and/or Southern blotting. In some aspects, the sample comprises, consists, or consists essentially of cells, tissue, an organ biopsy, an epithelial tissue, a lung, respiratory or airway tissue or organ, a circulatory tissue or organ, a skin tissue, bone tissue, muscle tissue, head, neck, brain, skin, bone and/or blood sample.

In yet another aspect, this disclosure provides an isolated T-cell exhibiting higher than or lower than baseline expression of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1.

In certain aspects, the T cells as disclosed herein comprise, consist of, or consist essentially of CD8+ T cells or CD4+ T-cells. In some aspects, the T cells comprise, consist of, or consist essentially of tissue-resident memory (TRM) cells. In certain aspects, the TRMs are TRMs expressing high levels of one or more of the genes set forth herein, or set forth on the accompanying Figures. In certain embodiments, the one or more genes are selected from: CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1. In a further aspect, the T cells are mammalian cells, e.g., human T cells.

Also disclosed herein is an agent or antibody that targets a cell exhibiting higher than or lower than baseline expression of one or more genes set forth herein, or set forth in the accompanying Figures. The one or more comprise, consist of, or consist essentially of CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1. In one aspect, the baseline expression is normalized mean gene expression. In another aspect, the higher than baseline expression is at least about a 2-fold increase in expression relative to baseline expression and/or lower than baseline expression is at least about a 2-fold decrease in expression relative to baseline expression. In a further aspect, the cells are tissue-resident memory cells (T_{RM}) or CD4⁺ T-cells. In one particular embodiment, the agent is an antibody, a small molecule, a protein, a peptide, a ligand mimetic or a nucleic acid.

In some aspects, the antigen specific cells comprise, consist of, or consist essentially of T cells. In one aspect, the T cells comprise, consist of, or consist essentially of CD4+ T-cells or CD8+ T-cells. In some aspects, the T cells comprise, consist of, or consist essentially of tissue resident memory (TRM) cells. In some aspects the T cells are CD4⁺ T_{RM} cells. In certain aspects, the T cells or TRM cells are cytotoxic.

In some aspects, the subject suffers from an autoimmune or fibrotic disorder or asthma. In some aspects, the autoimmune or fibrotic disorder or asthma is characterized in that T cells of the subject, once bound to an antigen, express higher or lower than baseline expression levels of the one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1. In certain embodiments, the agents disclosed herein target these cell populations expressing higher or lower than baseline expression levels of the one or more genes within the subject, leading to the depletion of the cells expressing higher or lower than baseline expression levels of the one or more genes. In certain embodiments, the cells express higher than baselines expression levels of the one or more genes. In some aspects, the one or more genes comprise, consist of, or consist essentially of AMICA1.

In yet another aspect, disclosed herein is a method of treating asthma or an autoimmune or fibrotic disorder in a subject, comprising, consisting of, or consisting essentially of administering an isolated T cell or population of isolated T cells comprising, consisting of, or consisting essentially of an antibody or antigen binding fragment that targets one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1. In some aspects, administration of the isolated T cell or population of T cells depletes cells expressing the one or more genes in the subject. In some aspects, the cells expressing the one or more genes comprise, consist of, or consist essentially of CD4+ T-cells or CD8+ T-cells. In some aspects, the cells expressing the one or more genes comprise, consist of, or consist essentially of tissue resident memory (TRM) cells. In some aspects the T cells are CD4⁺ or CD8+ T_{RM} cells. In certain aspects, the T cells or TRM cells are cytotoxic.

Further provided herein is a method of diagnosing a subject for autoimmune or fibrotic disease or conditions, inflammatory disease, and/or aberrant immune responses, including asthma, comprising, or consisting essentially of, or yet further consisting of contacting a sample isolated from the subject with an agent that detects the presence of one or more genes set forth set forth herein, or set forth in the accompanying Figures, wherein the presence of the one or more genes at higher or lower than baseline expression levels is diagnostic of autoimmune or fibrotic disease or conditions, inflammatory disease, and/or aberrant immune responses, including asthma. In one aspect, the method comprises, or consists essentially of, or yet further consists of contacting tissue-resident memory cells (TRMs) isolated from the subject with an antibody or agent, or one or more antibody or agent, that recognizes one or more genes selected from the group of CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, AND UMAP1, to determine the frequency of TRMs expressing the one or more genes recognized/bound from the sample, wherein a high frequency of one or more of these TRMs is diagnostic of autoimmune or fibrotic disease or conditions, inflammatory disease, and/or aberrant immune responses, including asthma.

In another aspect, the method of diagnosing autoimmune or fibrotic disease or conditions, inflammatory disease, and/or aberrant immune responses, including asthma, in a subject comprises, or consists essentially of, or yet further consists of contacting tissue-resident memory cells (TRMs) isolated from the subject with an antibody or agent that recognizes and binds one or more proteins encoded by a gene set forth herein, or set forth in the accompanying Figures (including but not limited CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, UMAP1) to determine the frequency of TRMs expressing these proteins, wherein a high frequency of TRMs expressing these proteins is diagnostic of autoimmune or fibrotic disease or conditions, inflammatory disease, and/or aberrant immune responses, including asthma.

Additionally, disclosed herein is a method of determining the density of tissue-resident memory cells (TRMs) in a sample isolated from the subject likely to contain these cells, the method comprising, or consisting essentially of, or yet further consisting of measuring expression of one or more gene selected from the group set forth herein, or set forth in the accompanying Figures (including but not limited CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, UMAP1) in the sample thereof, wherein higher or lower than baseline expression indicates higher density of TRMs in the sample thereof.

Further provided herein is a method of determining prognosis of a subject having autoimmune or fibrotic disease or conditions, inflammatory disease, and/or aberrant immune responses, including asthma, comprising, or consisting essentially of, or yet further consisting of measuring the density of tissue-resident memory cells (TRM) in a sample isolated from the patient, wherein a low density of TRM indicates a more positive prognosis. In one aspect, the method of prognosis of a subject having autoimmune or fibrotic disease or conditions, inflammatory disease, and/or aberrant immune responses, including asthma, comprises, or consists essentially of, or yet further consists of contacting tissue-resident memory cells (TRMs) isolated from the subject with an antibody or agent that recognizes and binds one or more genes selected from the group set forth herein, or set forth in the accompanying Figures, (including but not limited CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, UMAP1) to determine the frequency of TRMs expressing the one or more genes recognized/bound from the sample, wherein a high frequency of one or more of these TRMs indicates a more positive prognosis. In another aspect, the method of prognosis of a subject having autoimmune or fibrotic disease or conditions, inflammatory disease, and/or aberrant immune responses, including asthma, comprises, or consists essentially of, or yet further consists of contacting tissue-resident memory cells (TRMs) of the sample with an antibody or agent that recognizes and binds one or more proteins encoded by a gene set forth in the genes set forth herein, or set forth in the accompanying Figures (including but not limited CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, UMAP1) to determine the frequency of TRMs expressing these proteins, wherein a low frequency of TRMs expressing these proteins indicates a more positive prognosis.

In yet a further aspect, the method of determining prognosis of a subject having autoimmune or fibrotic disease or conditions, inflammatory disease, and/or aberrant immune responses, including asthma, comprises, or consists essentially of, or yet further consists of contacting tissue-resident memory cells (TRMS) isolated from the subject, with an antibody or agent that recognizes and binds CD103 to determine the frequency of CD103⁺ T_{RMS} or an antibody or agent that recognizes and binds a protein encoded by a gene set forth in the genes set forth herein, or set forth in the accompanying Figures (including but not limited CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, UMAP1) to determine the frequency of TRMs expressing the protein, wherein a low frequency of TRMS expressing the protein indicates a more positive prognosis. In a separate aspect, the method of determining prognosis of a subject having autoimmune or fibrotic disease or conditions, inflammatory disease, and/or aberrant immune responses, including asthma, comprises, or consists essentially of, or yet further consists of measuring the density of CD103 or proteins encoded by one or more gene set forth in the genes set forth herein, or set forth in the accompanying Figures (including but not limited CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, UMAP1) in the sample, wherein a low density of proteins indicates a more positive prognosis.

The sample can be contacted with an agent, optionally including a detectable label or tag. In one aspect, the detectable label or tag can comprise, or consist essentially of, or yet further consist of a radioisotope, a metal, horseradish peroxidase, alkaline phosphatase, avidin or biotin. In another aspect, the agent can comprise, or consist essentially of, or yet further consist of a polypeptide that binds to an expression product encoded by the gene, or a polynucleotide that hybridizes to a nucleic acid sequence encoding all or a portion of the gene. The polypeptide may comprise, or consist essentially of, or yet further consist of an antibody, an antigen binding fragment thereof, or a receptor that binds to the gene. In one aspect, the antibody is an IgG, IgA, IgM, IgE or IgD, or a subclass thereof. In another aspect, the IgG antibody is an IgG₁, IgG₂, IgG₃ or IgG₄. The antigen binding fragment can be a Fab, Fab′, F(ab′)₂, Fv, Fd, single-chain Fvs (scFv), disulfide-linked Fvs (sdFv) or V_{L} or V_{H}. In one aspect, the agent is contacted with the sample in conditions under which it can bind to the gene it targets.

The methods of this disclosure the method comprise, or consist essentially of, or yet further consist of detection by immunohistochemistry (IHC), in-situ hybridization (ISH), ELISA, immunoprecipitation, immunofluorescence, chemiluminescence, radioactivity, X-ray, nucleic acid hybridization, protein-protein interaction, immunoprecipitation, flow cytometry, Western blotting, polymerase chain reaction, DNA transcription, Northern blotting and/or Southern blotting. The sample may comprise, or consist essentially of, or yet further consist of cells, tissue, an organ biopsy, an epithelial tissue, a lung, respiratory or airway tissue or organ, a circulatory tissue or organ, a skin tissue, bone tissue, muscle tissue, head, neck, brain, skin, bone and/or blood sample.

Finally, provided herein is a kit comprising, or consisting essentially of, or yet further consisting of one or more of the isolated T-cells and/or the composition of this disclosure and instructions for use. In one aspect, the instruction for use provide directions to conduct any of the methods described herein.

### BRIEF DESCRIPTION OF THE DRAWINGS

FIGS. 1A to 1H show a non-limiting example of single-cell transcriptomic analysis revealing heterogeneity among airway CD4⁺ T cells. (A), Study overview. (B), Uniform manifold approximation and projection (UMAP) visualization of Seurat-based clustering analysis of 27,771 single-cell transcriptomes of ex vivo sorted CD4⁺ T cells obtained from 9 mild and 16 severe asthmatic patients. Each dot represents a cell and is shaded based on cluster type. Proportion of cells in each cluster is shown (parenthesis). (C), Heatmap of row-wise z-score-normalized mean expression of significantly enriched transcripts in each cluster. (D), Row-wise z-score-normalized mean expression and percent of expressing cells (size scale) plot for a selection of marker genes in each cluster. (E), UMAP shows TRM signature score (gray scale) for each cell. Clusters are delineated by lines. (F), GSEA between CD103⁺ T_{RM} cluster (top) and CD103⁻ T_{RM} cluster (bottom) versus all non-T_{RM} clusters using published TRM signature gene list. (G), Violin plot displays normalized expression of ITGAE (CD103) in TRM clusters (CD103⁺ T_{RM} and CD103⁻ T_{RM}) compared to T_{CM} cluster. (H), Violin plots show normalized expression of CD103 and CD69 expression in T_{RM} clusters compared to T_{CM} cluster (analysis done for 6 severe asthmatic patients).

FIGS. 2A to 2E show a non-limiting example of how a CD103⁺ T_{RM} cluster is increased in the airways of male severe asthmatics. (A), UMAP visualization of Seurat clustering analysis shown in FIG. 1B, distributed between 4 groups of patients based on sex and disease severity (equal cell numbers). Pie charts show cell proportions for each cluster per group. Cells are shaded based on cluster type. (B), Dot plots show proportions of CD103⁺ T_{RM} (top) and CD103 TRM (bottom) clusters for the 4 clinical groups (MA-mild asthma; SA=severe asthma). Each dot is data from a single subject. Data are mean±/−SEM; Mann-Whitney U test was used to compute significance for comparisons. **P<0.01; ns, non-significant. (C), Correlation plots between proportions of cells in CD103⁺ T_{RM} (%) (top) and CD103− TRM (bottom) cluster with clinical features (asthma severity score and 100%-post-bronchodilator FEV1/FVC %). Dots are shaded based on disease group. Spearman correlation coefficient r and the exact P value are shown. (D), Representative example of T_{RM} populations gated by flow cytometry from one donor (left) and dot plot (right) showing proportions of CD103⁺ T_{RM} cells in males (n=17) and females (n=13) per disease (mean±/−SEM, Mann-Whitney U test, *P<0.05; ***P<0.001). (E), Correlation plots between proportions of CD103⁺ T_{RM} cells (%) and clinical features (asthma severity score and 100%−post-BD FEV1/FVC %) in males and females. Dots are shaded based on disease group.

FIGS. 3A to 3G shows a non-limiting example of how a CD103⁺ T_{RM} subset displays features linked to cytotoxicity and TCR activation. (A), Volcano plot shows false discovery rate (FDR) (−log₁₀ adjusted P-value) and log₂ (fold change) in expression for genes differentially expressed in CD103⁺ T_{RM} versus CD103⁻ T_{RM} clusters. Dots are shaded according to the mean of expression (log₂) and sized based on the difference is the percentage of cells expressing the given gene, both derived from the group in which the gene is up-regulated. Gray dotted lines represent the statistical threshold values: log₂ (fold change)>0.25 and −log₁₀(FDR>1.3 (adjusted P-value<0.05). (B), Violin plots show normalized expression for genes up-regulated in CD103⁺ T_{RM} cluster. Shading code represents the fraction of cells expressing the indicated gene in each cluster. (C), GSEA shows enrichment of genes linked to cytotoxicity between TRM clusters (left); and between the CD103⁺ T_{RM} cluster from male and female severe asthmatics (right). q, false discovery rate. (D), Ingenuity pathway analysis (IPA) network of cytotoxicity-linked genes with increased expression (adjusted P-value<0.01 and log₂ (fold change)>0.5) in CD103⁺ T_{RM} cluster compared to CD103⁻ T_{RM} clusters. Arrows represent type of interaction between molecules and network biology, molecules are shaded based on their relative expression, and shaped based on molecule function. (E), IPA shows top 10 pathways enriched for genes with increased expression in CD103⁺ T_{RM} cluster compared to CD103-TRM cluster. Numbers show matching genes from dataset and IPA gene lists. (F), GSEA shows enrichment of genes linked to TCR signaling in CD103⁺ T_{RM} cluster compared to CD103⁻ T_{RM} clusters. q, false discovery rate. (G), Violin plots show normalized expression for genes up-regulated in CD103⁺ T_{RM} cluster. Shading code represents the fraction of cells expressing the given gene in each cluster.

FIGS. 4A to 4D shows a non-limiting example of how molecules that restrain T cell activation and effector functions are reduced in severe asthmatics. (A), Scatter plot shows the log₂ (fold change) expression of genes between severe and mild asthma in males (x-axis) and females (y-axis). Equal numbers of cells were used per group. Dotted lines indicate the statistical threshold value of fold change for gene filtering (adjusted P-value<0.05 and log₂ (fold change)>0.25). (B), Plot shows row-wise z-score-normalized mean expression and percent of expressing cells (size scale) for indicated genes in each cluster per disease. (C), GSEA plot shows enrichment of genes linked to CAMP immunoregulation pathway in cells from severe compared to mild asthmatic, in males (left) and females (right). (D), Violin plots show normalized expression for genes down-regulated in severe asthma in the T_{FH} (top) and TREG (bottom) cluster. Shading code represents the fraction of cells expressing the indicated gene in each cluster.

FIGS. 5A to 5C shows a non-limiting example of pro-inflammatory cytokines being expressed by airway CD4⁺ T cells from severe asthmatics. (A), UMAP visualization of 27,583 single-cell transcriptomes of sorted stimulated CD4⁺ T cells obtained from 14 mild and 16 severe asthmatic patients (top) and single-cell TRM signature score (bottom). Equal numbers of cells are shown in each group (n=20,518). UMAPs (left) show expression level of GZMB, CCL3, CCL4, IL13, IL4, IFNG, IL17A, IL21, and TNF transcripts in mild and severe asthma upon stimulation. Violin plots (right) show expression level in resting and stimulation condition from mild and severe asthmatic patients. Shading indicates percentage of cells expressing the indicated transcript. (B), Violin plots show expression for chemokine and cytokine genes in filtered GZMB-expressing CD4⁺ T cells. (C), Scatter plots show co-expression of CCL3 and IFNG with other cytokine genes transcripts in GZMB-expressing CD4⁺ T cells in resting and stimulation condition. Percentage of co-expressing cells is indicated (top right). Each dot represents one cell. Cells are shaded based on density value. Dotted lines indicate threshold of Seurat normalize gene expression (>0).

FIGS. 6A to 6I shows a non-limiting example of single-cell clustering analysis using Seurat. (A), Scatter plot represents cumulative standardized variance for genes (n=11,055) expressed in more than 0.1% of the CD4⁺ T cells (n=27,771) and with a UMI mean>0.01. Genes are ranked from highest to lowest standardized variance values. Genes below the dotted line were selected (n=809) as part of the highest variable genes explaining 15% of the cumulative standardized variance. Genes are shaded based on the UMI mean expression given by Seurat. (B), Scatter plot represents the distribution of genes ordered based on their UMI mean expression values in function of their individual standardized variance values. Genes shaded darker are the 809 highest variable genes with a UMI mean expression value>0.01. (C), Scatter plot shows the standardized variation for first 40 principal components (PCs) using the 809 most variable genes. Vertical lines indicate the number of PCs selected for clustering analysis in respect of Seurat methodology. (D), Violin plot shows distribution of number of genes per cell (thresholds: lower=200, upper-6,000) for each cluster. Shades are based on cluster-type. (E), Violin plot shows distribution of number of UMI per cell (threshold<30,000) for each cluster. Shades are based on cluster-type. (F), Violin plot shows distribution of the percentage of mitochondrial genes detected per cell (threshold<15%) for each cluster. Shades are based on cluster-type. (G), Heatmap with normalized enrichment score between each cluster with the rest of the cells. Shading scale corresponds to normalized enrichment scores for each gene list and clusters. Gray indicates no statistical significance (adjusted P-value>0.05). (H), GSEA for apoptosis signature in TAPOPTOSIS cluster versus all clusters. An enrichment of more than 0.5 was threshold value of exclusion of cluster from analysis. (I), Violin plots show normalized Seurat gene expression for cluster specific genes compared to an aggregation of remaining cells.

FIGS. 7A to 7E shows a non-limiting example of correlation of single-cell cluster proportions with clinical features. (A), Dot plot shows the percentage of CD103⁺ T_{RM} cells for each donor separated in mild and severe asthma groups. *P<0.05. (B), Dot plots show the proportions of T_{CM}, T_{REG}, T_{FH}, T_{IFNR}, T_{CYCLE}, and T_{CTL} for each donor, grouped per disease severity and sex. (A, B), Horizontal line, mean; error bar, SEM. Mann-Whitney U test was used to compute significance between disease severity. *P<0.05. Each dot is data from a single subject. (C), Heatmap shows Spearman correlation indices calculated between cluster proportions, grouped by sex, with clinical features. Shade code is based on correlation coefficient r and the exact P-values were computed using Spearman correlation. (D), Scatter plots show correlation between CD103⁺ T_{RM} proportions in each donor and specific clinical features (age at bronchoscopy and age of asthma diagnosis) in males (triangles) and females (circles). (E), Correlation scatter plots between percentage of CD103⁺ T_{RM} cells and percentage of T_{REG} cells (left) for each patient as well as percentage of T_{REG} cells for each patient and post-BD FEV1/FVC (right), in males and females. (D, E), Dots shaped based on patient sex, and Shaded based on diseases severity status. Correlation coefficient r and exact P value were computed using Spearman correlation.

FIGS. 8A to 8D shows a non-limiting example of flow cytometry gating strategy to isolate CD4⁺ T cells and subsets. (A), Flow cytometry gating strategy to sort live (Propidium iodide (PI), singlets (Width vs Area forward scatter (FSC)), lymphocyte size (Side scatter vs Forward scatter), CD3⁺ CD45⁺, CD4⁺ CD8⁻, CXCR5⁻, CD25⁻ CD127⁺, CD69^{+/-} CD103^{+/-}. (B), Dot plots showing percentage of lymphocytes, CD3⁺CD45⁺, CD4⁺, T_{FH}, T_{REG}, T_{EFF}, and CD103⁻ T_{RM} subsets per patient distributed between sex (shapes) and diseases groups. Horizontal line, mean; error bars, SEM. Mann-Whitney U test was used to compute significance for comparisons. *P<0.05; **P<0.01. (C), Normalized stacked bar charts represent proportions of the different CD4⁺ T cell clusters per donor grouped as mild and severe asthma. Sex is indicated on top of each bar (triangle-male, circle-female). Shades correspond to cluster type. (D), Pie charts represent average proportions of CD4⁺ T cell subsets in mild and severe asthma patient separated by sex (MA=mild asthma, SA=severe asthma). Shades correspond to cluster type.

FIGS. 9A to 9C shows a non-limiting example of expression of differentially expressed genes in CD103⁺ T_{RM}. (A), Heatmap of sorted bulk RNA-seq samples shows row-wise z-scored expression of 210 differentially expressed genes between CD103⁺ T_{RM}, CD103⁻ T_{RM}, and non-T_{RM} subsets. Adjusted P-value<0.05 and log₂ (fold change)>1. (B), Dot plots show normalized expression for example genes differentially up-regulated in CD103⁺ T_{RM} cells linked to tissue residency, cytotoxicity, and inflammation. Horizontal line, mean; error bar, SEM. (C), Scatter plots show log₂ (fold change) of gene expression between mild and severe asthma in male (x-axis) and female (y-axis) patients in each cluster. Dotted lines indicate the statistical threshold values of fold change for gene filtering.

FIGS. 10A to 10G shows a non-limiting example of single cell analysis of CD4⁺ T cells upon stimulation. (A), Scatter plot shows cumulative standardized variance of highly expressed (UMI mean>0.01) genes (n=11,287) ordered based on increasing individual standardized variance, within the stimulated CD4⁺ T cells (n=27,583). Genes are ranked from highest to lowest standardized variance values. Genes below the dotted lines represent the number of highest variable genes explaining 15% of the cumulative standardized variance (n=375). Genes are Shaded based on the UMI mean expression given by Seurat. (B), Scatter plot represents the distribution of genes ordered based on their UMI mean expression values in function of their individual standardized variance values. Gray dots show the most variable genes with a UMI mean expression value>0.01 representing 15% of the variance. (C), Violin plots show distribution of number of genes per cell (thresholds: lower=200, upper=6,000), number of UMI per cell (threshold=30,000), and percentage of mitochondrial genes per cell (<15%) for all stimulated CD4⁺ cells. (D), Volcano plot shows statistical significance [−log₁₀ (adjusted P value)] in function of the fold change (log₂) in expression between severe versus mild asthma cell after stimulation. Gray dotted lines represent the threshold value for fold change [Y-axis, log₂ (|fold change|)>0.25] and significance [x-axis, −log₁₀(adjusted P value)>2]. FDR, false discovery rate. (E), UMAPs (left) show Seurat-normalized expression level of CCL5, CCL20, CSF2, TNFSF14, and XCL1 transcripts in mild and severe asthma for all stimulated cells. Violin plots (right) show distributions of normalized expression [log₂ (CPM+1)] for given transcripts in resting and stimulation cells separated between disease status. Shade indicates percentage of cells expressing indicated transcript. (F), Volcano plot shows statistical significance (−log₁₀ adjusted P value) in function of the log₂-fold change in expression for differentially expressed genes when comparing expression between bulk RNA-seq stimulation versus resting within the CD103⁺ T_{RM} cells. Dots are shaded according to the average of expression (log₂) and sized on the basis of the fraction of cells expressing the given gene, both derived from the group in which the gene is up-regulated. Equal numbers of cells were sampled in each group. Gray dotted lines represent the threshold value for fold change [y-axis, log₂ (|fold change|)>1] and significance [x-axis, −log₁₀ (adjusted P value)>2]. FDR, false discovery rate. (G), Dot plots of genes differentially upregulated in stimulated CD103⁺ T_{RM} cells. Horizontal line, mean; error bar, SEM.

FIGS. 11A to 11C shows JAML expression is induced by cis-regulatory interactions between the CD3D and JAML promoters. (A), JAML expression (TPM) in resting and anti-CD3 and anti-CD28-stimulated CD4⁺ and CD8⁺ T cells from donors (n=104) from a published bulk RNA-seq dataset (Schmiedel et al., 2018). (B), ATAC-seq, ChIP-seq tracks and HiChIP interactions for the extended JAML and CD3 gene loci in indicated cell populations, the black arrow indicates the activation-induced intronic region. (C), CD3D expression (TPM) in resting and anti-CD3 and anti-CD28-stimulated CD4⁺ and CD8⁺ T cells from donors (n=104) from a published bulk RNA-seq dataset (Schmiedel et al., 2018).

FIG. 12 shows TCR signaling induces JAML expression in murine CD8+ T cells. ATAC-seq, ATAC-seq, ChIP-seq tracks and HiChIP interactions for the extended JAML and CD3 gene loci in indicated cell populations pertaining to (FIG. 11B).

FIGS. 13A to 13C shows TCR signaling induces JAML expression in human CD8+ T cells. FIG. 13A, FIG. 13B, Flow-cytometric analysis of anti-CD3 stimulated (A) or of anti-CD3+anti-CD28 or anti-CD3+anti-CXADR stimulated (B) CD4+ and CD8+ T cells, depicted is the expression of early activation markers CD69, CD25, 4-1BB and PD-1, data are shown as mean of duplicates from 4 individual donors (B). FIG. 13C shows Flow-cytometric analysis of anti-CD3+anti-CD28 or anti-CD3+anti-CXADR stimulated CD8+ T cells, depicted is the percentage of proliferated (Cell trace violet (CTV−)) cells.

FIGS. 14A to 14D shows JAML is functional in αβ T cells and is induced by TCR signaling. FIG. 14A, FIG. 14B, Flow-cytometric analysis of CD8+ T cells stimulated with anti-CD3+anti-CXADR, depicted is the expression of early activation markers CD69, CD25, 4-1BB and PD-1 (A) and secretion of pro-inflammatory cytokines interferon-g and tumor-necrosis factor-a (B). Depicted are the results for n=2 technical replicates (A,B). All data are representative of at least two independent experiments. (C), PCR analysis of JAML expression, depicted is the relative fold-change between the negative control guide RNA and the JAML targeting guide RNA. (D), Sanger-sequencing of CD8+ T cells, depicted is the wildtype allele (top, CRISPR targeting irrelevant gene sequence) and the CRISPR-modified allele (bottom, CRISPR targeting depicted JAML gene sequence).

FIGS. 15A to 15C shows JAML is highly expressed by CD8+ TILs in a murine melanoma model. (A), Representative histogram plots of in vitro stimulated CD8+ T cells showing the expression levels of JAML in CD8+ T cells treated as indicated. (B), Flow-cytometric analysis of murine CD8+ T cells stimulated with 0.1 ug/ml anti-CD3+indicated concentrations of anti-JAML, depicted is the expression of early activation markers CD69, CD25, PD-1 and 4-1BB, depicted are the results for n=2 technical replicates. (C), Flow-cytometric analysis of murine CD8+ T cells stimulated with 0.5 μg/ml of anti-CD3+indicated concentrations of anti-JAML, depicted is the expression of early activation markers CD69, CD25, PD-1 and 4-1BB, depicted are the results for n=3 technical replicates.

FIG. 16A shows cell types in skin tissue in atopic dermatitis. FIG. 16B shows JAML expression in T cells in skin tissue atopic dermatitis.

FIG. 17A shows cell types in esophagus tissue of patients with eosinophilic esophagitis. FIG. 17B, shows JAML expression in T cells from esophagus tissue of patients with eosinophilic esophagitis.

FIG. 18A shows cell types in colonic tissue of patients with ulcerative colitis. FIG. 18B shows JAML expression in T cells from colonic tissue of patients with ulcerative colitis

FIG. 19A shows cell types in ileum tissue of patients with Crohn's disease. FIG. 19B shows JAML expression in T cells from ileum tissue of patients with Crohn's disease.

FIG. 20 shows JAML expression in T cells in psoriatic arthritis. Clonally expanded T cells in synovial tissue express TRM markers (ZNF683, HLA-DR). These cells express high levels of JAML, which are increased in the joint when compared to levels expressed in blood.

FIG. 21A shows CD4 T cell types in synovial fluid samples from patients with juvenile arthritis. FIG. 21B shows JAML expression in CD4 T cells from synovial fluid of patients with juvenile arthritis.

FIG. 22A shows CD8 T cell types in synovial fluid samples from patients with juvenile arthritis. FIG. 22B shows JAML expression in CD8 T cells from synovial fluid of patients with juvenile arthritis.

FIG. 23A shows T cell types in synovial tissue samples from patients with rheumatoid arthritis. FIG. 23B shows JAML expression in T cells from synovial tissue of patients with rheumatoid arthritis

FIG. 24A shows T cell types in synovial tissue samples from patients with rheumatoid arthritis. FIG. 24B shows JAML expression in T cells from synovial tissue of patients with rheumatoid arthritis

FIG. 25A shows immune cell types in blood of patients with systemic lupus erythematosus. FIG. 25B shows JAML expression in T cells from patients with systemic lupus erythematosus

FIG. 26A shows immune cell types in blood of patients with Kawasaki disease. FIG. 26B shows JAML expression in T cells from patients with Kawasaki disease.

FIG. 27A shows cell types in lung tissue samples from patients with in pulmonary fibrosis. FIG. 27B shows JAML expression in T cells from lung tissue samples from pulmonary fibrosis.

FIG. 28A shows cell types in lung tissue samples from patients with pulmonary fibrosis. FIG. 28B shows JAML expression in T cells from lung tissue samples of patients with pulmonary fibrosis.

FIG. 29A shows cell types in lung tissue from patients with lung scleroderma. FIG. 29B shows JAML expression in T cells from patients with lung scleroderma.

FIG. 30A shows cell types in skin tissue from patients with scleroderma. FIG. 30B shows JAML expression in T cells from patients with skin scleroderma.

FIG. 31A shows cell types in liver tissue from patients with primary sclerosing cholangitis. FIG. 31B shows JAML expression in T cells from liver tissue of patients with primary sclerosing cholangitis.

FIG. 32A shows cell types in lung tissue from patients with in chronic obstructive pulmonary disease. FIG. 32B shows JAML expression in T cells from lung tissue of patients with chronic obstructive pulmonary disease.

FIG. 33A shows cell types in CSF samples from patients with Alzheimer's disease. FIG. 33B shows JAML expression in T cells from patients with Alzheimer's disease.

FIG. 34A to 34E shows CD8 TRM cells is the most abundant subset of T cells in severe asthma airway biopsies: (A) Study overview. (B) Uniform manifold approximation and projection (UMAP) visualization of Seurat-based clustering analysis of ≈25,000 single-cell transcriptomes of live cells obtained from dispersed broncho-biopsies collected from mild and severe asthmatic patients. Each dot represents a cell and is shaded based on cluster type. Proportion of cells in each cluster is shown as a cumulative bar chart on the right. (C) Heatmap of row-wise z-score-normalized mean expression of significantly enriched transcripts in each cluster. (D) Row-wise z-score-normalized mean expression and percent of expressing cells (size scale) plot for a selection of marker genes in each cluster. (E) UMAP shows normalized expression (Log 2 [CPM+1]) for 6 example genes characterizing cell type biology.

FIG. 35A to 35G shows CD8 T_{RM} cells in severe asthma are cytotoxic, pro-inflammatory, pro-fibrotic, and TCR expanded: (A) UMAP representation for only the cell surface marker CD8B⁺ cells selected by Cellular Indexing of Transcriptomes and Epitopes by Sequencing (CITE-seq) analysis (protein). (Top) Protein expression of T_{RM} cell surface markers (CD69 and CD103). (Bottom) Plots showing mRNA normalized expression (log₂ [CPM+1]) of 6 genes: 4 constitutively expressed T_{RM} markers genes (ITGAE coding for CD103, CD69, ITGA1 coding for the integrin CD49a, ZNF683 coding for the transcription factor HOBIT, AMICA1 coding for a T_{RM} activation co-stimulatory signal, and CCL5, a gene usually induced post CD8+ T cells activation with pro-inflammatory function coding for CCL5/RANTES). (B) Volcano plot shows statistical significance (False discovery rate, FDR, Y-axis −Log₁₀ adjP value) in function of the log₂-fold change in expression (x-axis) for all differentially expressed genes when comparing all single cells between disease groups (mild asthma on the left and severe asthma on the right. Main genes are indicated in larger font size. Dots are shaded according to the average of expression (log₂) and sized based on the fraction of cells expressing the given gene, both derived from the group in which the gene is up regulated. Equal numbers of cells were sampled in each group. Genes are shown with fold change [y-axis, log₂ (|fold change|)>1] and significance [x-axis, −log₁₀(adjusted P value)>2]. (C-D) Violin plots show normalized expression (log₂ [CPM+1]) for genes upregulated in severe asthma. Shaded code represents the fraction of cells expressing the indicated gene in both disease groups (MA=Mild asthma, SA=Severe asthma). The mean (central black line), median (black dot), standard deviation (box), and 5-95% percentile range (whiskers) are shown. (E) Dot plot showing percentage of GZMB⁺ AREG⁺ CD8⁺ T cells from total CD3⁺ T cells per patient distributed between diseases groups. Bars represent the mean and t-lines represent SEM (**, P<0.01; Kruskal-Wallis one-way test followed by Dunn's post-hoc test). (F) Scatter plots show co-expression, normalized expression [log₂ (CPM+1)], for AMICA1 transcripts (coding for the T_{RM} specific co-stimulatory molecule) with cytotoxic (GZMB, PRF1, GNLY) and pro-inflammatory (CCL4, CCL5) genes in non-stimulatory condition between disease groups (Mild, left; Severe, right). Percentage of co-expressing cells is indicated (top right). Each dot represents one cell. Cells are shaded based on density value. Dotted lines indicate threshold of Seurat normalize gene expression (>0). (G) Scatter plots show co-expression of score density for a list of published cytotoxic and T_{RM} genes between both disease groups (mild and severe asthma). Percentage of co-expressing cells is indicated (top right). Each dot represents one cell. Cells are shaded based on density value. Dotted lines indicate threshold of Seurat normalize gene expression (>0).

FIG. 36A to 36D: (A) UMAPs (left) show protein expression level of CD8B, CD69, CD103 and (right) RNA normalized expression of transcripts coding for molecules listed on the left (CD8B, CD69, ITGAE) in all biopsy cells. Shade scale indicates normalized level of expression (log₂ [CPM+1]). Left scale for protein by CITE-seq, and right scale for mRNA expression. (B) Scatter plots show co-expression, normalized expression [log₂ (CPM+1)], of GZMB with other cytotoxic (PRF1, GNLY) and pro-inflammatory (CCL4, CCL5) genes in non-stimulatory condition. Percentage of co-expressing cells is indicated (top right). Each dot represents one cell. Cells are shaded based on density value. Dotted lines indicate threshold of Seurat normalize gene expression (>0). (C) Scatter plots show co-expression, normalized expression [log₂ (CPM+1)], of the pro-fibrotic AREG gene with cytotoxic (GZMB, PRF1, GNLY) and pro-inflammatory (CCL4, CCL5) genes in non-stimulatory condition. Percentage of co-expressing cells is indicated (top right). Each dot represents one cell. Cells are shaded based on density value. Dotted lines indicate threshold of Seurat normalize gene expression (>0). (D) Violin plots show normalized expression (log₂ [CPM+1]) for genes downregulated in severe asthma. Shaded code represents the fraction of cells expressing the indicated gene in each cluster.

FIG. 37A to 37D shows “Luminal” CD8+T_{RM} cells in severe asthma display more cytotoxic, inflammatory, and innate like molecular features: (A) Volcano plot shows statistical significance (Y-axis False discovery rate, FDR, −Log₁₀ adjP value) in function of the Log₂-fold change in expression (x-axis) for differentially expressed genes when comparing CD8+ T cells, isolated from BAL, between disease groups (mild asthma on the left and severe asthma on the right). Few genes are shown. Dots are shaded according to the average of expression (log₂) and sized based on the fraction of cells expressing the given gene, both derived from the group in which the gene is up regulated. Equal numbers of cells were sampled in each group. Genes are shown with fold change [y-axis, log₂ (|fold change|)>1] and significance [x-axis, −log₁₀(adjusted P value)>2]. (B) Gene set enrichment analysis (GSEA) for multiple lists of published signature gene lists corresponding to molecular signaling pathways (cytotoxicity, glucocorticoids response, TCR signaling, survival/apoptosis, and immunomodulatory signaling) between disease groups of CD8⁺ T cells. RES=Relative enrichment score. (C) Violin plots show normalized expression (log₂ [CPM+1]), for differentially expressed genes between both disease groups (enriched in severe asthma, left; depleted in severe asthma, right). Shade code represents the fraction of cells expressing the indicated gene in each cluster. (D) Dot plots showing protein concentration for 2 pro-inflammatory molecules (CCL3 and CCL4) normalized to the total amount of protein from individual BAL supernatant. Data are separated based on disease groups (MA: Mild asthma, SA: Severe asthma; Luminex, multiplexed enzyme-linked immunosorbent assay). Bars represent the mean and t-lines represent SEM (*, P<0.05; Kruskal-Wallis one-way test followed by Dunn's post-hoc test).

FIG. 38A to 38G shows disease-specific heterogeneity in CD8 T cells from BAL of severe asthmatics: (A) UMAP visualization of Seurat-based clustering analysis of single-cell transcriptomes of CD8⁺ sorted T cells obtained from cellular fraction of broncho-alveolar lavages (BAL) collected from mild and severe asthmatic patients. Each dot represents a cell and is shaded based on cluster type. On the lower right corner are listed the name of clusters based on main biology revealed by analysis of Seurat top cluster differentiated genes. (B) Violin plots show normalized expression (log₂ [CPM+1]) for transcripts specifically enriched or depleted in T_{RM} cells for each cluster identified. Shade code represents the fraction of cells expressing the indicated gene in each cluster. Shaded bar on top of violins represents the different clusters listed in FIG. 4A. (C) Cumulative bar charts show the change of cluster proportions between both disease groups (MA=mild asthma; Sa=Severe asthma). Shade code corresponds to the cluster ID code shown in FIG. 4A. (D) Dot plots show donor specific proportion for clusters significantly enriched or depleted in severe asthma. Each dot is the proportion for a donor, bars represent the mean, and t-lines represent SEM (*, P<0.05; **, P<0.01, Kruskal-Wallis one-way test; ns=non-significant). (E) Heatmap of row-wise z-score-normalized mean expression of significantly enriched transcripts in each of the 9 T_{RM} enriched clusters. Shaded bar on top of violins represents the different clusters listed in FIG. 4A. (F) Row-wise z-score-normalized mean expression and percent of expressing cells (size scale) plot for a selection of marker genes in each cluster. Shaded bar on top of violins represents the different clusters listed in FIG. 4A. (G) Gene set enrichment analysis (GSEA) for multiple lists of published signature genes corresponding to molecular signaling pathways (cell cycle, cytotoxicity, glucocorticoids (CS) response, cell fitness/survival, type I & II signaling pathway, and intrinsic immunomodulatory signaling) between cluster enriched for a given list and all other clusters or another specific cluster. RES=Relative enrichment score.

FIG. 39A to 39E shows UMAP visualization of single-cell transcriptomes of sorted stimulated CD8⁺ T cells from broncho-alveolar lavages obtained from (A) mild (MA, black dots) and severe (SA, gray dots) asthmatic patients and (B) single-cell T_{RM} signature score. (D) UMAPs (left) show normalized expression level (Log₂ [CPM+1]) of GZMB, CCL3, CCL4, IL6ST, IFNG, TNF, and XCL1 transcripts between mild (left) and severe (right) asthma upon 2 hours ex vivo stimulation of BAL cells. Equal numbers of cells are shown in each group. (E) Violin plots show normalized expression level (Log₂ [CPM+1]) of GZMB, CCL3, CCL4, IL6ST, IFNG, TNF, and XCL1 transcripts in resting (left) and stimulation (right) conditions from mild (1^{st} and 3^{rd} violins) and severe (2^{nd} and 4^{th} violins) asthmatic patients. Shade indicates percentage of cells expressing the indicated transcript.

### DETAILED DESCRIPTION

Throughout this disclosure, various publications, patents and published patent specifications are referenced by an identifying citation or by an Arabic numeral, the complete bibliographic citation for which is found preceding the claims. The disclosures of these publications, patents and published patent specifications are hereby incorporated by reference into the present disclosure to more fully describe the state of the art to which this disclosure pertains.

The practice of the present disclosure employs, unless otherwise indicated, techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996))

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions (i.e., stringent hybridization conditions).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “X” or “X” as provided herein includes any of the recombinant or naturally-occurring forms of X, also known as X, or variants or homologs thereof that maintain X activity (e.g. within at least 50%, 80%, 90%, 95%, 96%, 97%, 98%, 99% or 100% activity compared to X). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring X protein polypeptide. In embodiments, X protein is the protein as identified by the UniProt reference number X, or a variant, homolog or functional fragment thereof. In aspects, X includes the amino acid sequence of SEQ ID NO:X. In aspects, X has the amino acid sequence of X. In aspects, X has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:X.

Antibodies are large, complex molecules (molecular weight of ~150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F (ab)′₂, a dimer of Fab which itself is a light chain joined to V_{H}-C_{H1} by a disulfide bond. The F (ab)′₂ may be reduced under mild conditions to break the disulfide linkage in the hinge region, thereby converting the F (ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The epitope of a mAb is the region of its antigen to which the mAb binds. Two antibodies bind to the same or overlapping epitope if each competitively inhibits (blocks) binding of the other to the antigen. That is, a 1×, 5×, 10, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “multimer” refers to a complex comprising multiple monomers (e.g. a protein complex) associated by noncovalent bonds. The monomers be substantially identical monomers, or the monomers may be different. In embodiments, the multimer is a dimer, a trimer, a tetramer, or a pentamer. Thus, a trimer comprises three monomers associated by noncovalent bonds.

A “ligand” refers to an agent, e.g., a polypeptide or other molecule, capable of binding to a receptor or antibody, antibody variant, antibody region or fragment thereof.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The terms “virus” or “virus particle” are used according to their plain ordinary meaning in the biological arts and refer to a particle including a viral genome (e.g. DNA, RNA, single strand, double strand), a protective coat of proteins (e.g. capsid) and associated proteins, and in the case of enveloped viruses (e.g. herpesvirus), an envelope including lipids and optionally components of host cell membranes, and/or viral proteins. In embodiments, the virus is an Arenavirus.

The terms “multiplicity of infection” or “MOI” are used according to its plain ordinary meaning in Virology and refers to the ratio of components to the target (e.g., cell) in a given area. In embodiments, the area is assumed to be homogenous.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods. Thus, a recombinant protein refers to a protein made by introducing a cell with a nucleic acid that is not typically found in the cell (e.g. non-native DNA). The cells containing the non-native nucleic acid may then transcribe and translate the protein.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “bind” and “bound” as used herein is used in accordance with its plain and ordinary meaning and refers to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc.;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

As defined herein, the term “inhibition”, “inhibit”, “inhibiting” and the like in reference to cell proliferation (e.g., cancer cell proliferation) means negatively affecting (e.g., decreasing proliferation) or killing the cell. In embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein.

The term “associated” or “associated with” in the context of a substance or substance activity or function associated with a disease (e.g., arenavirus infection) means that the disease is caused by (in whole or in part), or a symptom of the disease is caused by (in whole or in part) the substance or substance activity or function. Alternatively, the substance may be an indicator of the disease. Thus, an associated substance may serve as a means of targeting disease tissue.

A “therapeutic agent” as referred to herein, is a composition useful in treating or preventing a disease such as a viral infection (e.g. Lassa fever). In embodiments, the therapeutic agent is an anti-viral agent. “Anti-viral agent” is used in accordance with its plain ordinary meaning and refers to a composition (e.g. compound, drug, antagonist, inhibitor, modulator) having anti-viral properties or the ability to inhibit viral infection. In embodiments, an anti-viral agent targets a viral protein. In embodiments, an anti-viral agent inhibits viral entry into a host cell. In embodiments, an anti-viral agent inhibits replication of viral components. In embodiments, an anti-viral inhibits release of viral particles. In embodiments, an anti-viral inhibits assembly of viral particles.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

In certain non-limiting embodiments, the condition, disease, or disorder capable of being treated by the aspects disclosed herein is an autoimmune or fibrotic disorder, disease, or condition, proinflammatory condition, or an aberrant immune response. In certain embodiments, the autoimmune or fibrotic disorder, disease, or condition, proinflammatory condition, or an aberrant immune response is selected from the group consisting of polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, nonalcoholic fatty liver disease, primary biliary cholangitis, autoimmune hepatitis, autoimmune kidney disease, chronic obstructive pulmonary disease (COPD), Castleman's disease, asthma (mild intermittent, mild persistent, moderate persistent, or severe persistent), allergic asthma (mild intermittent, mild persistent, moderate persistent, or severe persistent), allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenia purpura, pernicious anemia, sensorineural hearing loss, idiopathic bilateral progressive sensorineural hearing loss, autoimmune polyglandular syndrome type I or type II, immune infertility and immune-mediated infertility. The term “prevent” refers to a decrease in the occurrence of a disease or disease symptoms in a patient. As indicated above, the prevention may be complete (no detectable symptoms) or partial, such that fewer symptoms are observed than would likely occur absent treatment. In one aspect, the term “treatment” excludes “prevention.”

As used herein, a “symptom” of a disease includes any clinical or laboratory manifestation associated with the disease, and is not limited to what a subject can feel or observe.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from binding assays or cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

“Co-administer” it is meant that a composition described herein is

administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols. The preparations may also be combined with inhaled mucolytics (e.g., rhDNase, as known in the art) or with inhaled bronchodilators (short or long acting beta agonists, short or long acting anticholinergics), inhaled corticosteroids, or inhaled antibiotics to improve the efficacy of these drugs by providing additive or synergistic effects. The compositions of the present invention can be delivered transdermally, by a topical route, formulated as applicator sticks, solutions, suspensions, emulsions, gels, creams, ointments, nanoparticles, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In embodiments, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus). In the context of cancer vaccine, the vaccine composition can provide mRNA encoding certain peptides that are associated with cancer, e.g. peptides that are substantially exclusively or highly expressed in cancer cells as compared to normal cells. The subject, after vaccination with the cancer vaccine composition, can have immunity against the peptides that are associated with cancer and kill the cancer cells with specificity.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the agents provided herein including embodiments thereof, augments the agent's immune response. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### MODES OF CARRYING OUT THE DISCLOSURE

Disclosed herein are T-cells exhibiting higher than or lower than baseline expression of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1. In a further aspect, the T cells are mammalian cells, e.g., human T cells.

In certain aspects, the T cells as disclosed herein comprise, consist of, or consist essentially of CD8+ T cells or CD4+ T-cells. In some aspects, the T cells comprise, consist of, or consist essentially of tissue-resident memory (TRM) cells. In certain aspects, the TRMs are TRMs expressing high levels of one or more of the genes set forth herein, or set forth on the accompanying Figures. In certain embodiments, the one or more genes are selected from: CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1.

Disclosed herein are methods of decreasing, reducing, or inhibiting the activity of an antigen specific cells, or cells that are bound to an antigen. In some aspects, the antigen specific cell expresses higher than baselines expression of one or more genes, or proteins associated with or expressed by one or more genes, comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1. In some aspects, the one or more genes comprise, consist of, or consist essentially of AMICA1.

In some aspects, the antigen specific cells comprise, consist of, or consist essentially of T cells. In one aspect, the T cells comprise, consist of, or consist essentially of CD4+ T-cells or CD8+ T-cells. In some aspects, the T cells comprise, consist of, or consist essentially of tissue resident memory (TRM) cells. In some aspects the T cells are CD4+ or CD8+ TRM cells. In certain aspects, the T cells or TRM cells are cytotoxic.

In some aspects, the methods deplete the T cells expressing the one or more genes expressed at higher than baseline levels of expression within the subject. In some aspects, the subject suffers from an autoimmune or fibrotic disorder or asthma. In some aspects, the autoimmune or fibrotic disorder or asthma is characterized in that T cells of the subject bind to an antigen and express a higher than baseline level of the one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1. In aspects disclosed herein, agents including but not limited to antibodies or antigen binding fragments, decrease, reduce, or inhibit the activity of, or deplete, the T cells expressing the one or more genes at a higher than baseline level within the subject, leading to the depletion of the cells.

In some aspects, the higher than baseline expression of the one or more genes is at least about a 1-fold increase in expression relative to baseline expression and/or lower than baseline expression of the one or more genes is at least about a 1-fold decrease in expression relative to baseline expression. In some aspects, the higher than baseline expression of the one or more genes is at least about a 2-fold increase in expression relative to baseline expression and/or lower than baseline expression of the one or more genes is at least about a 2-fold decrease in expression relative to baseline expression. Expression can be reduced or increased by at least about 2 or more, or about 3, or about 4, or about 5, or about 6, or about 7, or about 8, or about 9, or about 10, or about 11, or about 12, or about 13, or about 14, or about 15 fold as compared to a comparative wild-type cell. One of skill in the art can monitor expression of the genes using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

In another aspect, the antibody is an IgG, IgA, IgM, IgE or IgD, or a subclass thereof. The antibody can also be an IgG selected from the group of IgG1, IgG2, IgG3 or IgG4. Furthermore, the antigen binding fragment can be selected from the group of a Fab, Fab′, F(ab′)₂, Fv, Fd, single-chain Fvs (scFv), disulfide-linked Fvs (sdFv) or VL or VH.

### Compositions, Methods of Treatment, Diagnosis and Prognosis

For the disclosed methods, in one aspect, the one or more genes set forth herein, or set forth on the accompanying Figures. In another aspect, the one or more genes comprise, consist of, or consist essentially of CD103, CD69, ITGAE, CD69, ITGA1, AMICA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1. In some aspects, the one or more genes comprise, consist of, or consist essentially of AMICA1.

Also provided herein are methods to reduce or inhibit an immune response and treat conditions requiring selective immunotherapy, comprising, or consisting essentially of, or yet further consisting of, contacting a target cell with the compositions as described herein. The contacting can be performed in vitro, or alternatively in vivo, thereby providing immunotherapy to a subject such as for example, a human patient.

In one aspect, this disclosure provides a method of treating asthma or an autoimmune or fibrotic disease in a subject comprising, consisting of, or consisting essentially of administering to the subject an effective amount of an agent that induces higher than or lower than baseline expression of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in T-cells, thereby treating asthma or the autoimmune or fibrotic disease in the subject.

In one aspect, this disclosure provides a method of treating asthma or an autoimmune or fibrotic disease in a subject or sample comprising, consisting of, or consisting essentially of administering an effective amount of one or more of an agent that induces or inhibits in T-cells activity of one or more proteins encoded by genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 to the subject, thereby treating asthma or the autoimmune or fibrotic disease in the subject.

In some aspects, the active agent comprises, consists of, or consists essentially of an antibody, a small molecule, a protein, a peptide, a ligand mimetic or a nucleic acid. In certain aspects, the agent is capable of modulating the activity or expression of AMICA1. In certain other aspects, the agent is capable of blocking or inhibiting the activity of the protein expressed by AMICA1, JAML.

In some aspects of the methods disclosed herein, the T cells comprise, consist of, or consist essentially of CD4+ T-cells or CD8+ T-cells. In some aspects, the T cells comprise, consist of, or consist essentially of tissue resident memory (TRM) cells. In certain aspects, the T cells or TRM cells are cytotoxic. In some aspects, the T cells are autologous to the subject being treated.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials.

For the above methods, an effective amount is administered, and administration of the cell or population serves to attenuate any symptom or prevent additional symptoms from arising. When administration is for the purposes of preventing or reducing the likelihood of asthma or the recurrence of the autoimmune or fibrotic disorder, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

The methods provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies.

In other aspects, provided are one or more methods of diagnosing autoimmune disease or conditions, inflammatory disease, and/or aberrant immune responses, including asthma, identifying a subject likely to benefit from or respond to treatment, (including but not limited to immunotherapy (including anti-autoimmune disease or conditions, inflammatory disease, and/or aberrant immune responses, including asthma, or anti-asthma immunotherapy)), determining the effectiveness of treatment, and/or determining a prognosis of a subject having autoimmune disease or conditions, inflammatory disease, and/or aberrant immune responses, including asthma. The one or more methods comprise, or alternatively consist essentially of, or yet further consist of, detecting or measuring the population or amount of TRMs, or a sub-population of TRMs expressing high levels of one or more of the genes set forth herein, or set forth on the accompanying Figures, in the subject or in a sample isolated from the subject. In certain embodiments, a lower amount of TRMs or lower amount of the sub-population of TRMs expressing high levels of one or more of the genes set forth herein, or set forth on the accompanying Figures, in the subject or sample indicates that the subject is likely to benefit from or respond to treatment, that the treatment likely to be effective in the subject, or that the subject is likely to proceed have a positive clinical response. In certain embodiments, a higher amount of TRMs or higher amount of the sub-population of TRMs expressing high levels of one or more of the genes set forth herein, or set forth on the accompanying Figures, in the subject or sample indicates that the subject is less likely to benefit from or respond to treatment, that the treatment is likely not as effective in the subject as other therapies, or that the subject has a poor prognosis with available therapies.

In yet another aspect, this disclosure provides a method of diagnosing asthma or an autoimmune or fibrotic disease in a subject, comprising, consisting of, or consisting essentially of contacting a sample isolated from the subject with an agent that detects the presence of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in the sample isolated from the subject, wherein the presence of the one or more genes at higher or lower than baseline expression levels is a diagnostic indicator of asthma or the autoimmune or fibrotic disease or wherein the absence of the one or more genes at higher or lower than baseline expression levels is not diagnostic indicator of asthma or the autoimmune or fibrotic disease.

In one aspect, this disclosure provides a method of diagnosing asthma or an autoimmune or fibrotic disease in a subject comprising, consisting of, or consisting essentially of contacting tissue-resident memory cells (TRMs) isolated from the subject or a sample isolated from the subject, with an antibody or agent that recognizes and binds one or more proteins encoded by a gene comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 to determine the frequency of TRMs expressing these proteins, wherein a high frequency of TRMs expressing these proteins is diagnostic of asthma or the autoimmune or fibrotic disease.

In one aspect, this disclosure provides a method of determining the density of tissue-resident memory cells (TRMs) in a sample isolated from a subject comprising, consisting of, or consisting essentially of measuring expression of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in the sample, wherein higher or lower than baseline expression indicates higher density of TRMs in the sample thereof.

In one aspect, this disclosure provides a method of determining prognosis of a subject having an autoimmune or fibrotic e disease comprising, consisting of, or consisting essentially of measuring the density of tissue-resident memory cells (TRM) in a sample isolated from the subject, wherein a low density of TRM indicates a more positive prognosis or wherein a high density or TRM indicates a more negative prognosis optionally wherein the more negative prognosis comprises a decreased probability in the reduction of symptoms of the autoimmune or fibrotic disease and a lack of low density TRM indicates a more positive prognosis, optionally increased probability in the reduction of symptoms of the autoimmune or fibrotic disease.

In one aspect, this disclosure provides a method of determining prognosis of a subject suffering from an autoimmune or fibrotic disease comprising, consisting of, or consisting essentially of contacting tissue-resident memory cells (TRMs) isolated from the subject with one or more of: an antibody or agent that recognizes and binds one or more proteins encoded by a gene comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1, wherein a low density of TRMs indicates a more positive prognosis or wherein a high density of TRMs indicates a more negative prognosis, optionally wherein the more negative prognosis comprises a decreased probability in the reduction of symptoms of the autoimmune or fibrotic disease, and wherein the more positive prognosis comprises an increased probability in the reduction of symptoms of the autoimmune or fibrotic disease.

In one aspect, this disclosure provides method of determining prognosis of a subject having an autoimmune or fibrotic disease comprising, consisting of, or consisting essentially of contacting tissue-resident memory cells (TRMs) isolated from the subject with an antibody or agent that recognizes and binds one or more proteins encoded by a genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs expressing these proteins, wherein a low density of TRMs indicates a more positive prognosis or wherein a high density of TRMs indicates a more negative prognosis, optionally wherein the more negative prognosis comprises, consists of, or consists essentially of a decreased probability in the reduction of symptoms of the autoimmune or fibrotic disease, and wherein the more positive prognosis comprises, consists of, or consists essentially of an increased probability in the reduction of symptoms of the autoimmune or fibrotic disease.

In one aspect, this disclosure provides a method of determining prognosis of a subject having an autoimmune or fibrotic disease comprising, consisting of, or consisting essentially of contacting tissue-resident memory cells (TRMs) isolated from the subject with an antibody or agent that recognizes and binds CD103 to determine the frequency of CD103+TRMs or an antibody that recognizes and binds a protein encoded by a genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs expressing the protein, wherein a low density of TRMs indicates a more positive prognosis or wherein a high density of TRMs indicates a more negative prognosis, optionally wherein the more negative prognosis comprises, consists of, or consists essentially of a decreased probability in the reduction of symptoms of the autoimmune or fibrotic disease, and wherein the more positive prognosis comprises, consists of, or consists essentially of an increased probability in the reduction of symptoms of the autoimmune or fibrotic disease.

In one aspect, this disclosure provides a method of determining the responsiveness of a subject having asthma or an autoimmune or fibrotic disease to immunotherapy comprising, consisting of, or consisting essentially of contacting tissue-resident memory cells (TRMs) isolated from the subject with an antibody or agent that recognizes and binds one or more proteins encoded by a genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs in the subject, wherein a high frequency of TRMs indicates lack of responsiveness to immunotherapy.

In one aspect, this disclosure provides a method of determining the responsiveness of a subject having asthma or an autoimmune disease to immunotherapy comprising, consisting of, or consisting essentially of contacting tissue-resident memory cells (TRMs) isolated from the subject with an antibody or agent that recognizes and binds one or more proteins encoded by a genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 and, to determine the frequency of TRMs expressing these proteins, wherein a low frequency of TRMs expressing these proteins indicates responsiveness to immunotherapy.

In one aspect, this disclosure provides a method of determining prognosis of a subject having an autoimmune or fibrotic disease comprising, consisting of, or consisting essentially of measuring the density of CD103 or proteins encoded by one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in a sample isolated from the subject, wherein a low density of TRM indicates a more positive prognosis or wherein a high density or TRM indicates a more negative prognosis optionally wherein the more negative prognosis comprises, consists of, or consists essentially of a decreased probability in the reduction of symptoms of the autoimmune or fibrotic disease and a lack of low density TRM indicates a more positive prognosis, optionally increased probability in the reduction of symptoms of the autoimmune or fibrotic disease.

In one aspect, this disclosure provides a method of identifying a subject that will or is likely to respond to asthma therapy or an autoimmune or fibrotic disease therapy, comprising, consisting of, or consisting essentially of contacting a sample isolated from the subject with an agent that detects the presence of one or more genes comprising, consisting of, or consisting essentially of AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 in the sample, wherein the presence of the one or more genes at higher or lower than baseline expression levels indicates that the subject is likely to respond to the asthma therapy or the autoimmune or fibrotic disease therapy.

The T-cells, population of T-cells, active agent and/or compositions provided herein may be administered either alone or in combination with diluents, known anti-cancer therapeutics, and/or with other components such as cytokines or other cell populations that are immunostimulatory. They may be administered as a first line therapy, a second line therapy, a third line therapy, or further therapy. Non-limiting examples of additional therapies include chemotherapeutics or biologics. Appropriate treatment regimens will be determined by the treating physician or veterinarian

The cells of this disclosure can be a mammalian cell, humans, non-human primate cells (e.g., apes, gibbons, chimpanzees, orangutans, monkeys, macaques, and the like), domestic animal cells (e.g., dogs and cats), horses, cows, goats, sheep, pigs, mouse, rat, rabbit, guinea pig).

The methods are useful to treat subjects such as humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). The cells can be autologous or allogeneic and can be the same or different species than the subject being treated. A mammal can be any age or at any stage of development (e.g., an adult, teen, child, infant, or a mammal in utero). A mammal can be male or female. In certain embodiments the subject has or is suspected of having an autoimmune or fibrotic disease or disorder. In one aspect, the animal is treated as an animal model for a particular patient or tumor type, or can be used to assay combination therapies.

### Kits

Finally, provided herein is a kit comprising, or consisting essentially of, or yet further consisting of one or more of the isolated T-cells and/or the composition of this disclosure and instructions for use. In one particular aspect, the present disclosure provides kits for performing the methods of this disclosure as well as instructions for carrying out the methods of the present disclosure.

The kits are useful for diagnosing an autoimmune or fibrotic disorder or asthma in a subject from a biological sample taken from the subject e.g., any bodily fluid including, but not limited to, e.g., sputum, serum, plasma, lymph, cystic fluid, urine, stool, cerebrospinal fluid, acitic fluid or blood and including biopsy samples of body tissue. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### EXAMPLES

Severe asthma (SA) is characterized by persistent airway inflammation, airway hyper-responsiveness (AHR) and remodeling¹⁻³. It poses a substantial health, economic, and societal burden causing significant morbidity, poor quality of life, and even death⁴⁻⁶. The evolving understanding of severe asthma as a clinically diverse collection of phenotypes and endotypes, alongside the stratified prescription of novel biological asthma therapies reflect underlying pathophysiological and molecular heterogeneity^{7,8} For instance, phenotypes stratified by sex and age of asthma onset have shown clinical importance⁹. The immune cellular compartment of the asthmatic airways has been shown to drive airway inflammation and be responsible for the pathophysiology of disease¹⁰. The persistent inflammation driven pathophysiology leads to active remodeling of the structural cells in the airways thus developing chronic disease. Although asthma has been associated with a type 2 (T2) immune response, referred to as “T2 disease”, such as CD4⁺ T helper (T_{H}) 2 cells¹¹ and type 2 innate lymphoid cells (ILC2)^{12,13}, the complexity of the cellular and molecular pathways involved in asthmatic airways have mired current therapeutics especially in the treatment-unresponsive severe disease patients¹⁴. Indeed, despite usage of high dose of corticosteroids, and biologics against T2 inflammation, limited treatment efficacy can be observed amongst a fraction of severe asthmatic patients¹⁵⁻¹⁹. Non-T_{H}2 driven asthma contributes to a significant proportion of patients who respond poorly to therapeutics as well²⁰. This emphasizes the difficult to control, spectrum of severe asthma disease and the need to investigate the treatment unresponsive pathological immune mechanisms persisting in severe asthma, usually resulting in years of poorly managed disease.

To reveal the pathophysiological differences in associated asthma disease, invasive surgical lung biopsies have been conducted, however with limited studies on live patients focusing on the distal lung regions of severe asthmatic patients²¹. To address this limitation, the inventors sought to define the immune and structural/remodeling differences in the distal airway compartment of mild and severe asthma patients with a history of treatment, from collected lavage and endobronchial biopsies. To provide biological insight on the possible mechanistic drivers of disease severity, the inventors undertook an unbiased transcriptomic analysis of immune and structural cells isolated from human endobronchial biopsies of the inflamed conducting airway mucosal tissue, along with bronchoalveolar lavage cellular infiltrate from patients with mild and severe asthma. From this approach, a comprehensive description of both immune and structural airway cells and their possible contributions to severity of disease was developed.

Patients with severe uncontrolled asthma represent a distinct endotype with persistent airway inflammation and remodeling that is refractory to corticosteroid treatment. To determine T cell subsets and effector molecules that drive pathogenesis of severe asthma, the inventors performed single-cell transcriptome analysis of >50,000 airway CD4⁺ T cells isolated from bronchoalveolar lavage (BAL) samples from patients with mild and severe asthma. Striking heterogeneity was observed in the nature of CD4⁺ T cells present in asthmatics' airways with tissue-resident memory (T_{RM}) cells making a dominant contribution. Notably, in severe asthmatics a subset of CD4⁺ T_{RM} cells (CD103-expressing) was significantly increased, comprising nearly 65% of all CD4+ T cells in the airways of male patients with severe asthma when compared to mild asthma (13%). This subset was enriched for transcripts linked to T cell receptor (TCR) activation (HLA-DRB1, HLA-DPA1, CD40LG) and cytotoxicity (GZMB, GZMH), and following stimulation expressed high levels of transcripts encoding for pro-inflammatory non-T_{H}2 cytokines (CCL3, CCL4, CCL5, TNF, LIGHT) that could fuel persistent airway inflammation and remodeling.

These findings indicate the need to look beyond the traditional T2 model of severe asthma to better understand the heterogeneity of this disease.

### Example 1: Single-Cell Transcriptomic Analysis Reveals Heterogeneity Among Airway CD4⁺ T Cells in Asthma

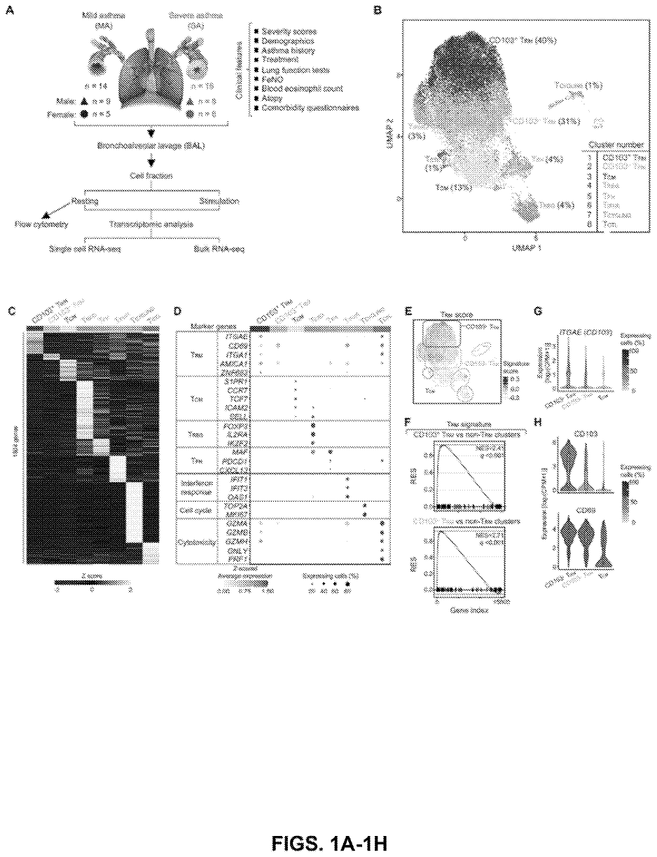

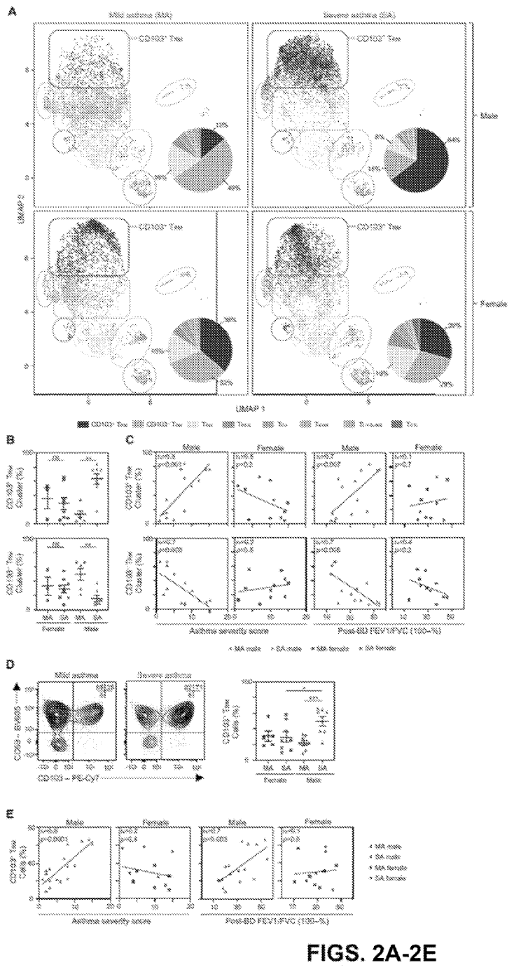

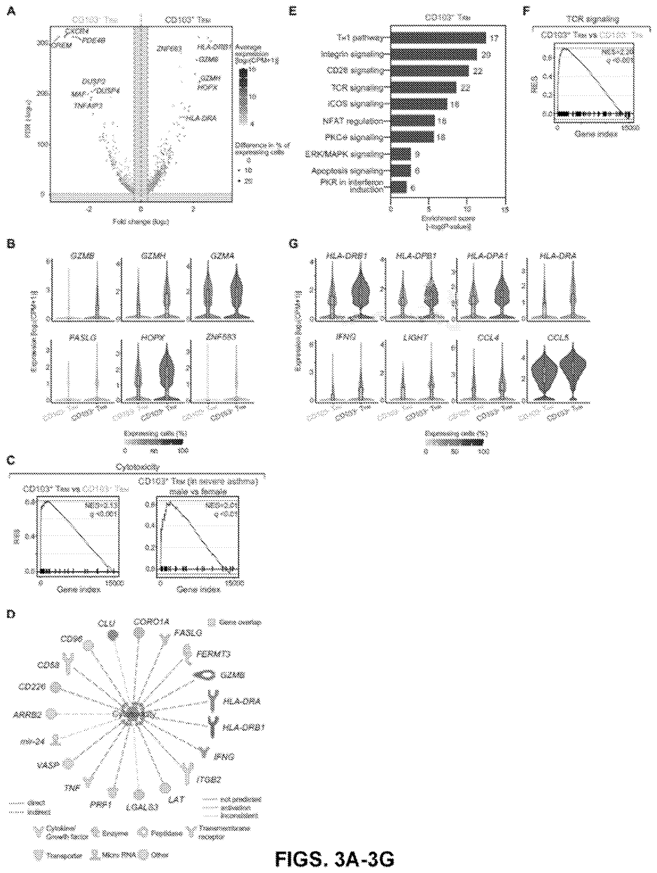

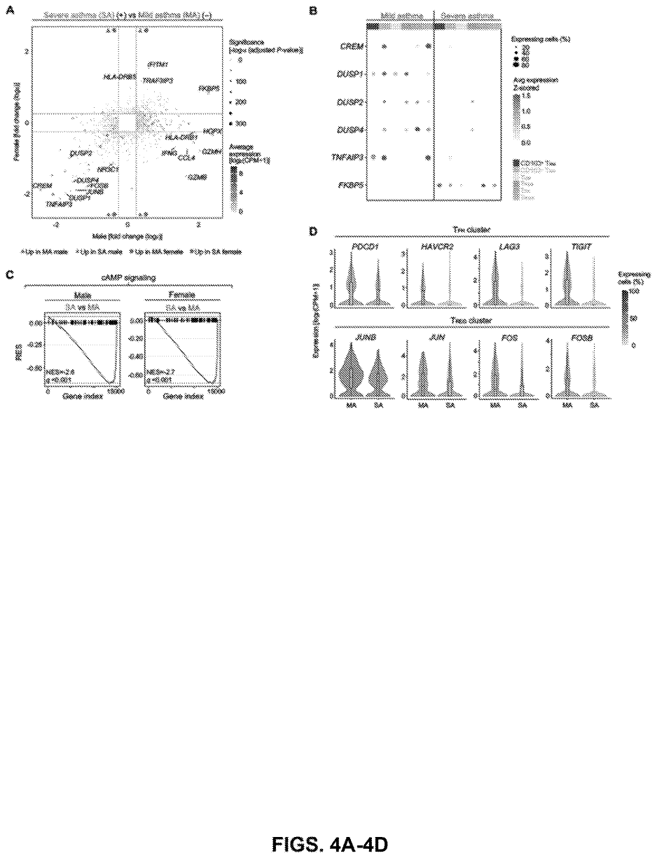

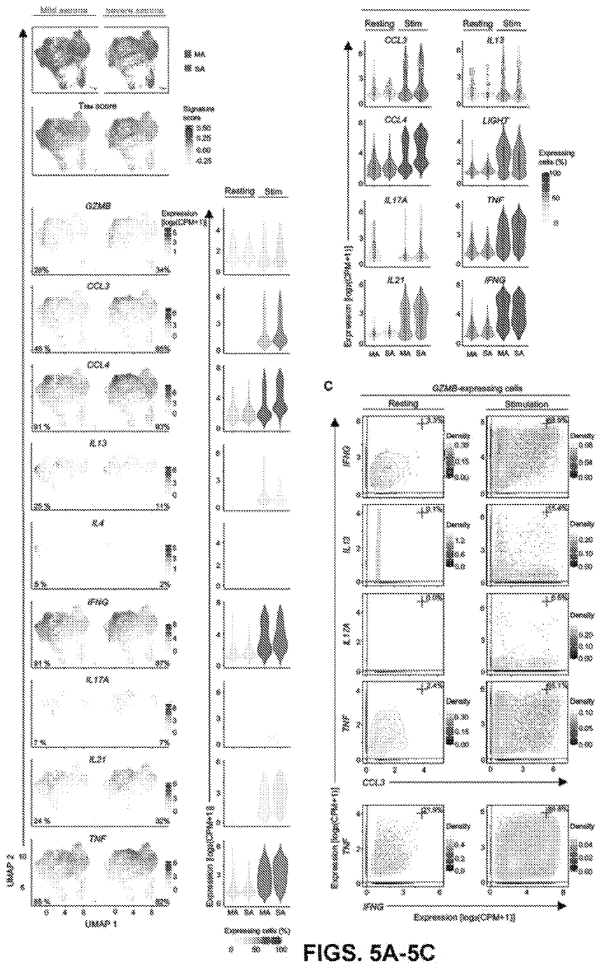

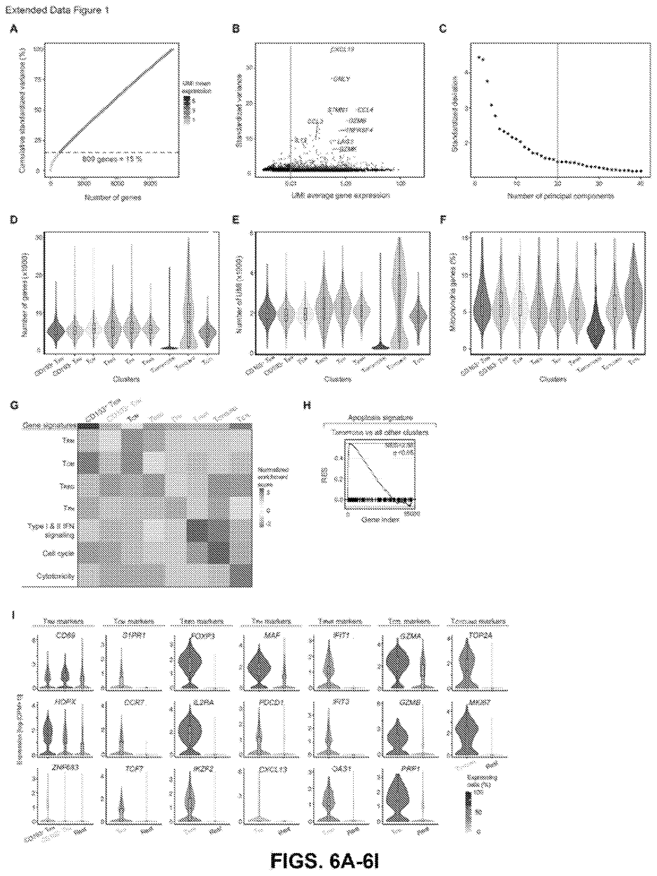

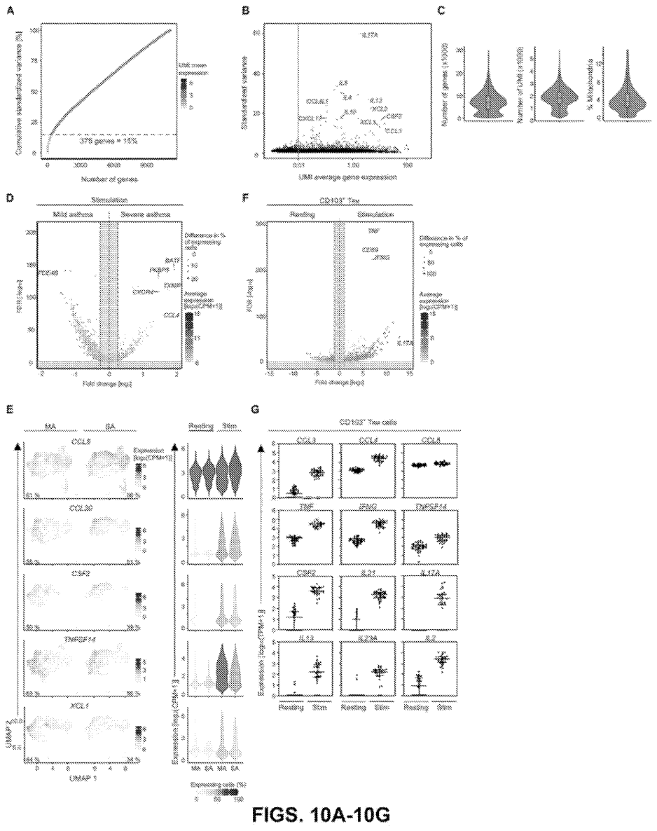

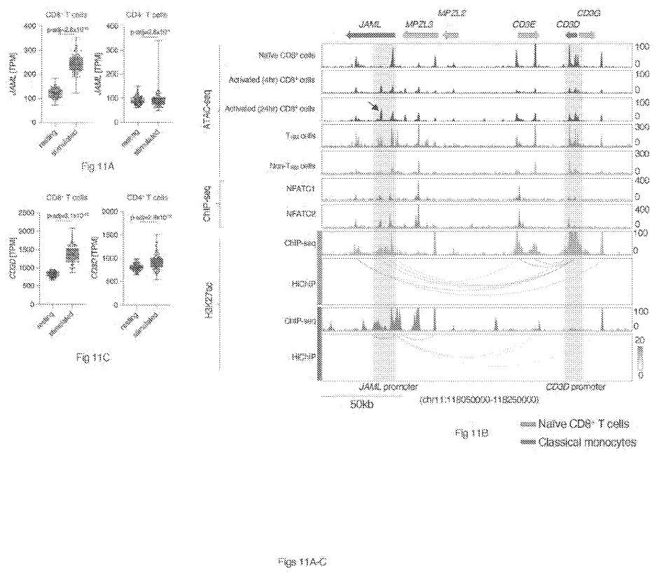

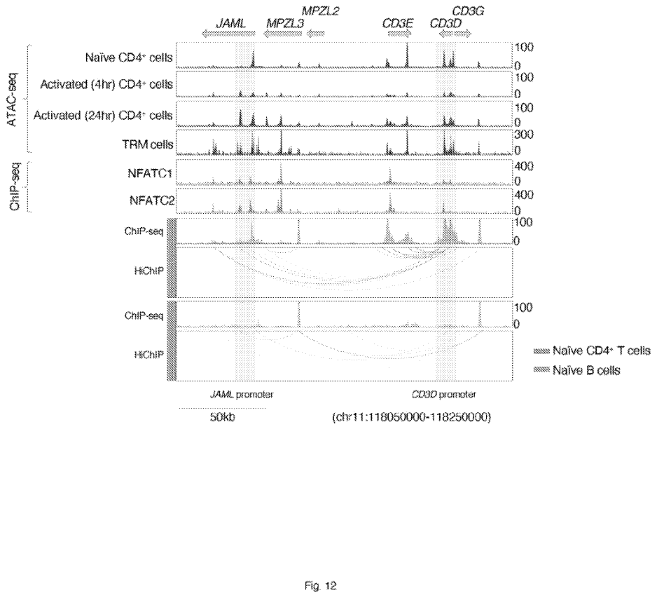

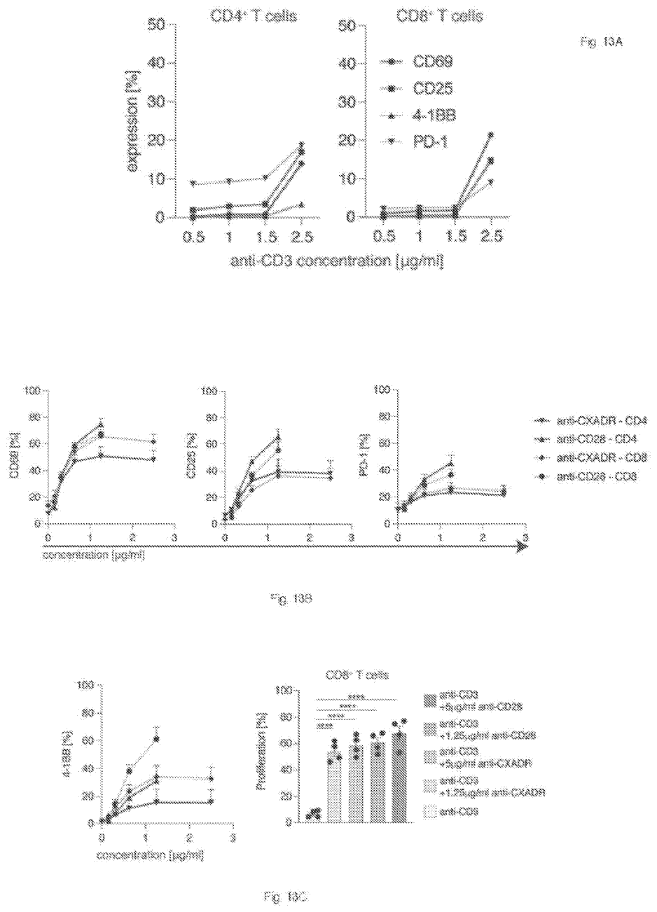

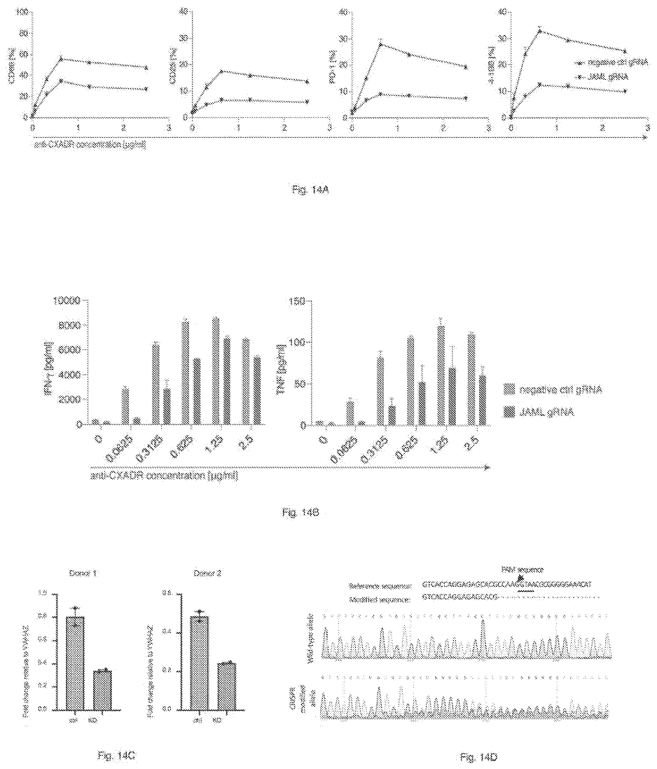

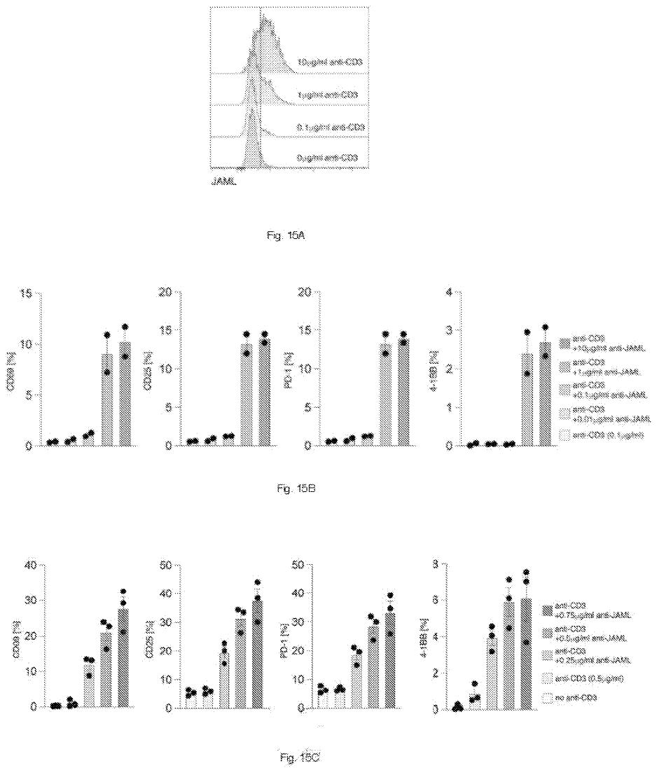

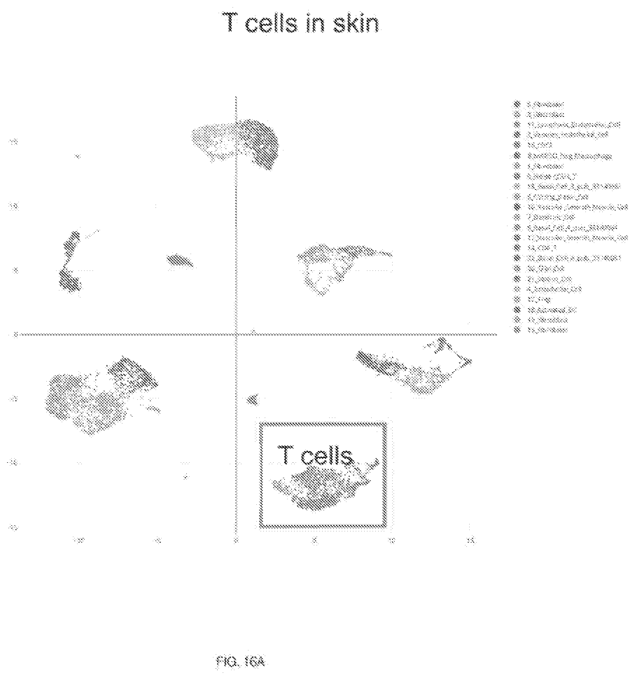

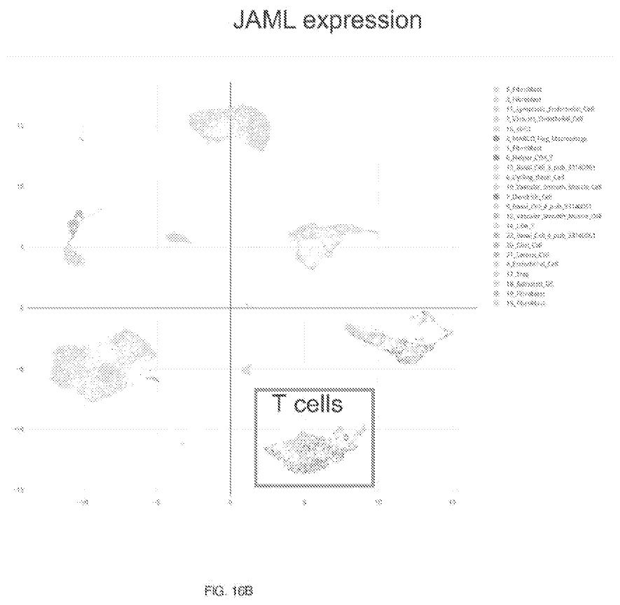

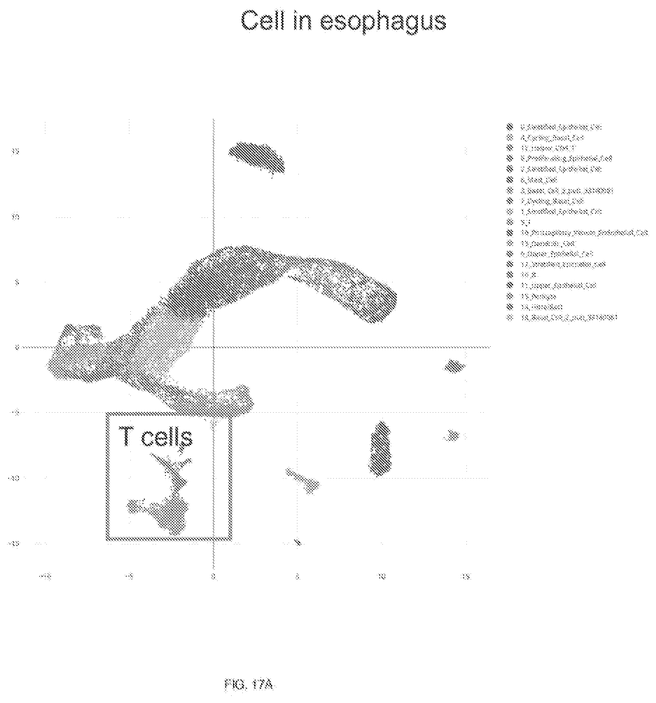

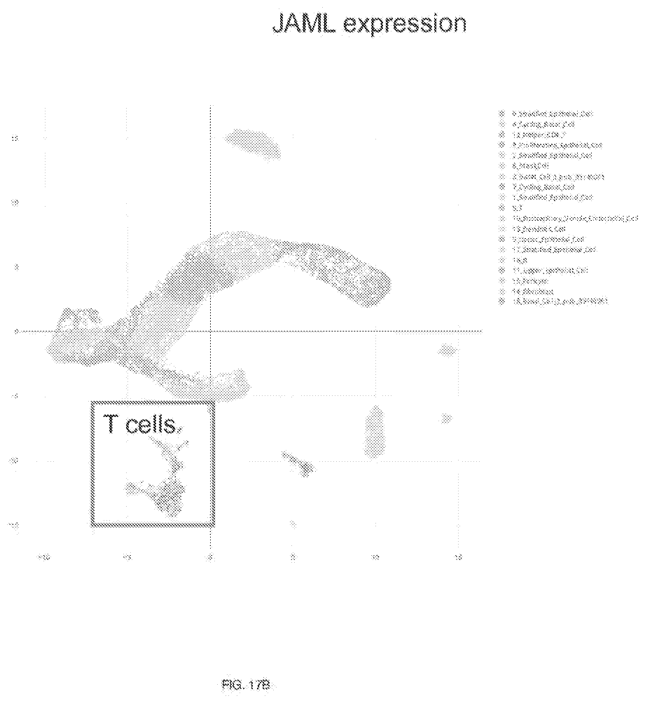

Patients with severe asthma (n=16, 50% male) were enrolled from the Wessex AsThma CoHort of difficult asthma (WATCH) study(31), which has extensively characterized patients with severe asthma requiring ‘high dose’ and/or ‘continuous or frequent use of oral corticosteroid’, in accordance with global initiative of asthma (GINA) management step 4 and 5(32, 33) (FIG. 1A). As controls, the inventors enrolled subjects with mild asthma (n=14, 64% male) as the inventors' aim was to define the airway CD4⁺ T cell subsets specifically associated with asthma disease severity. The inventors isolated and purified CD4⁺ T cells (n>40,000) from bronchoalveolar lavage (BAL) specimens and performed single-cell RNA-seq assay using the oil droplet-based platform (10× Genomics). After exclusion of doublets and low-quality transcriptomes, clustering analysis of airway CD4⁺ T cells (n=27,771 cells; median ~1,304 cells/donor) revealed 8 transcriptionally distinct subsets (see Methods, FIG. 1B, FIG. 6A-F).

The molecular identity for each cluster was established based on enrichment analysis of marker and signature genes linked to established CD4⁺ T cell subsets (FIGS. 1C and D, FIGS. 6G and H,). The two largest clusters (cluster 1 and 2), representing 71% of all CD4⁺ T cells analyzed, were significantly enriched for T_{RM} signature genes(30, 34, 35) (FIGS. 1E and F, and FIGS. 6G and I), and thus were defined as T_{RM} cells. Notably, both T_{RM} clusters expressed high levels of the T_{RM} marker gene CD69(35) (FIG. 6I), whereas T_{RM} cells in cluster 1 expressed higher levels of another T_{RM} marker gene, ITGAE (FIG. 1G), which encodes for the alpha chain of the integrin CD103, a transmembrane protein required for the adhesion of T cells to E-cadherin expressed by epithelial cells(34, 36, 37). By labeling BAL cells with anti-CD69 and anti-CD103 DNA-barcoded antibodies prior to performing single-cell RNA-sequencing, the inventors confirmed that CD69 expression was increased in both T_{RM} clusters, whereas CD103 surface expression was mainly observed in cluster 1, indicating enrichment of CD103-expressing T_{RM} cells in this cluster (FIG. 1H); henceforth, for simplicity, the inventors refer to cluster 1 and 2 as CD103⁺ T_{RM} and CD103⁺ T_{RM} clusters, respectively.

The third largest cluster (cluster 3=13% of airway CD4⁺ T cells) showed significant negative enrichment of T_{RM} signature genes and was characterized by higher expression of CCR7 and SELL transcripts, which encode for cell-surface markers linked to central memory T cells (T_{CM}, FIG. 1D, and FIGS. 6G and I). In addition, those cells were expressing transcripts linked to tissue egression (SIPR1, ICAM2)(34, 38, 39), and stemness (TCF7)(40) (FIG. 1D and FIG. 61I); thus cluster 3 represented T_{CM} cells. Cluster 4 (4%) and cluster 5 (4%) cells were enriched for transcripts encoding marker genes linked to regulatory T cells (T_{REG}) (FOXP3, IL2RA, IKZF2)(41, 42) and T_{FH} cells (CXCL13, PDCD1, MAF)(43), respectively (FIG. 1D and FIG. 6I). Cluster 6 (3%) was highly enriched for type I and II interferon response genes like IFIT1, IFIT3, and OAS1, reminiscent of the recently described interferon response genes expressing helper T cell subset (T_{H}IFNR) with a potential regulatory function in allergy(44) (FIG. 1D and FIG. 6I). Two other relatively small clusters of cells, cluster 7 (1%), enriched for transcripts linked to cell cycle (MKI67, TOP2A), represented proliferating cells(45), and cluster 8 (1%), which expressed high levels of several transcripts encoding for cytotoxicity molecules (GNLY, PRF1, GZMB), represented cytotoxic CD4⁺ T cells(46) (FIG. 1D and FIG. 6I). Overall, these results highlight a substantial level of heterogeneity in the profile of CD4⁺ T cells present in the asthmatic airways, with T_{RM} subsets making up la major contribution.

### Example 2: CD103⁺ T_{RM} Cells are Significantly Increased in the Airways of Males with Severe Asthma

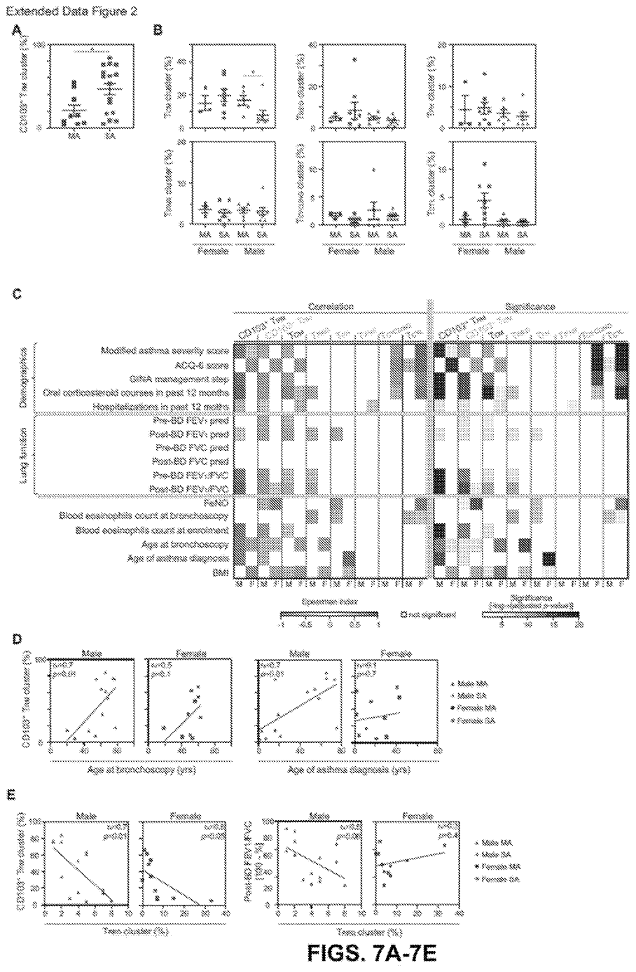

The inventors next determined the CD4⁺ T cell subsets that were increased in the airways of patients with severe asthma and assessed their association with clinical and physiological parameters of asthma severity. Because biological sex has been shown to stratify severe asthmatics into distinct endotypes, where males with severe asthma display poor lung function and high use of maintenance corticosteroids(47, 48), Applicant also explored the influence of biological sex on the composition of airway CD4⁺ T cells in severe asthma. Among the CD4⁺ T cell subsets, the proportion of cells in the CD103⁺ T_{RM} subset was significantly increased in the airways of patients with severe asthma compared to mild asthma (46% versus 21%) (FIG. 7A), while the proportions of CD103⁻ T_{RM} subset was significantly decreased (FIGS. 2A and B). The increase in CD103⁺ T_{RM} subset was significant only when comparing male subjects with severe and mild asthma (FIG. 2B; 64% versus 13% of all CD4⁺ T cells in severe versus mild asthma).

The proportions of cells in CD103⁻ T_{RM} and T_{CM} cluster were significantly lower in male severe asthmatics (FIG. 7B). All other airway CD4⁺ T cell subsets (with >1% cells) showed no significant differences between mild and severe asthmatics in both males and females (FIG. 7B).

To investigate the strength of the association between asthma severity and CD103⁺ T_{RM} subset in males, the inventors assessed their correlations with a range of asthma severity-related parameters in a sex-specific manner (FIG. 7C). In males but not female asthmatics, the inventors also found a significant positive correlation between the proportion of cells in CD103⁺ T_{RM} subset and asthma severity score (a composite score ranging from 0-20, adapted from Severe Asthma Research Program(49), comprising of symptoms, quality of life, degree of airflow obstruction, use of corticosteroids or biologicals, and frequency of asthma exacerbations requiring oral corticosteroids and hospitalization (FIG. 2C, FIG. 7C). The inventors also found a significant positive correlation directly with the severity of airflow obstruction (post-bronchodilator FEV₁ and FEV₁/FVC) in males (FIG. 2C, FIG. 7C). Notably, the proportions of airway TREG cells was negatively correlated with the severity of airflow obstruction in males (FIGS. 7C and E), suggesting a potential imbalance between CD103⁺ T_{RM} and T_{REG} subset in the airways of males with severe asthma. The inventors found that age was positively correlated with proportions of CD103⁺ T_{RM} subset in airways of male severe asthmatics (FIGS. 7C and D). The fact that 7 of the 8 male subjects studied developed late-onset severe asthma (age >40 years), suggests that older age and late-onset of disease may also be linked to this specific immune profile observed in males with severe asthma (FIGS. 7C and D).

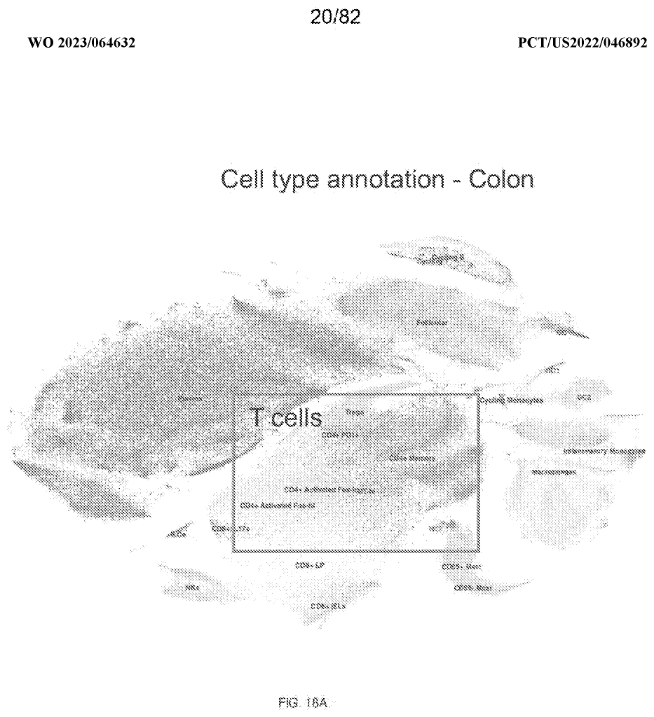

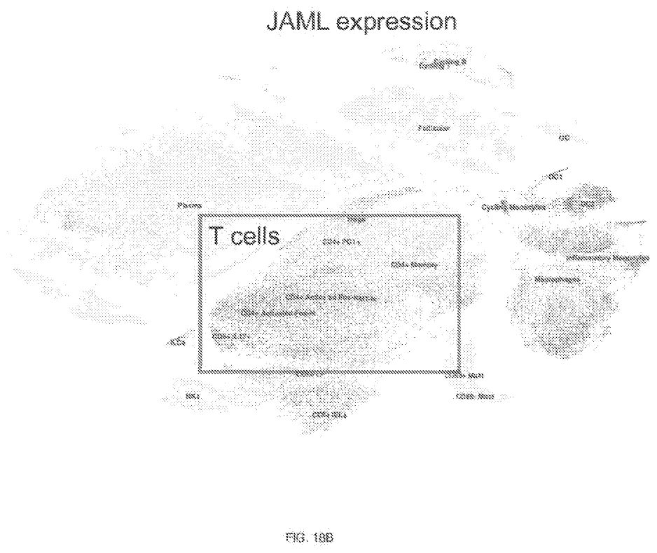

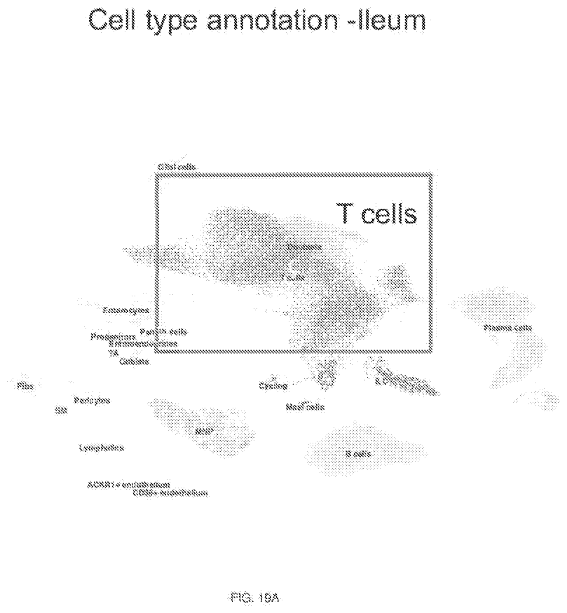

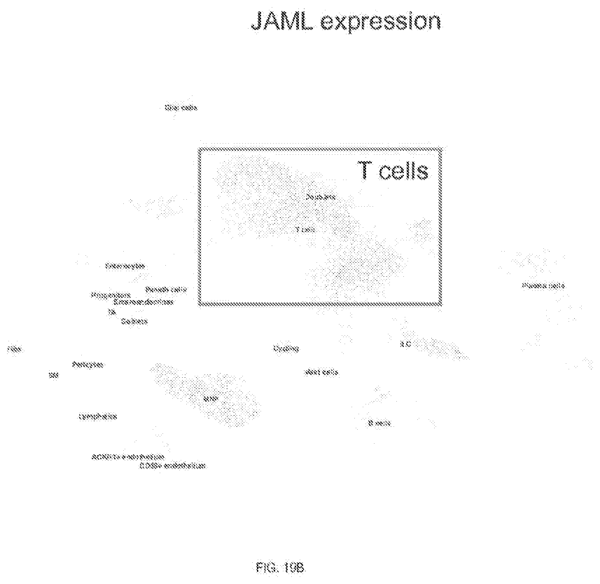

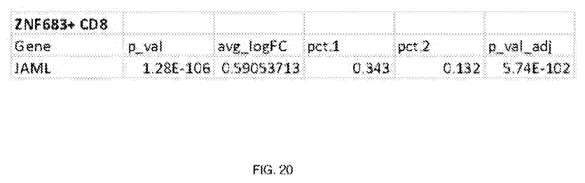

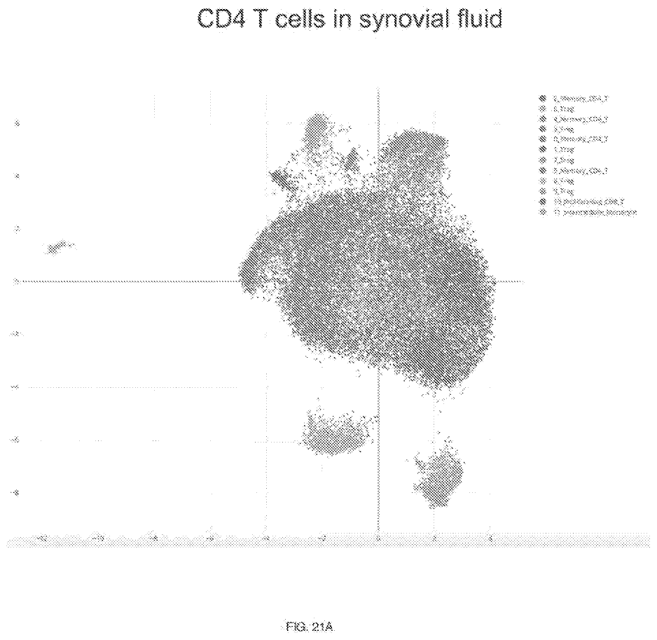

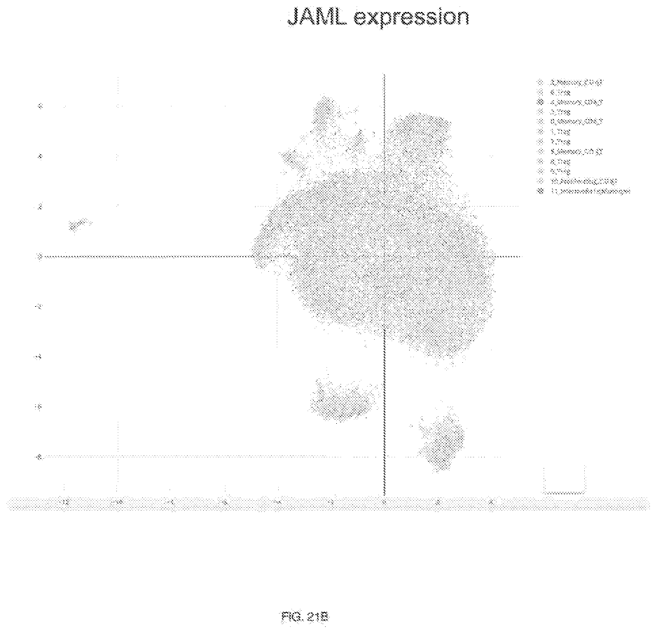

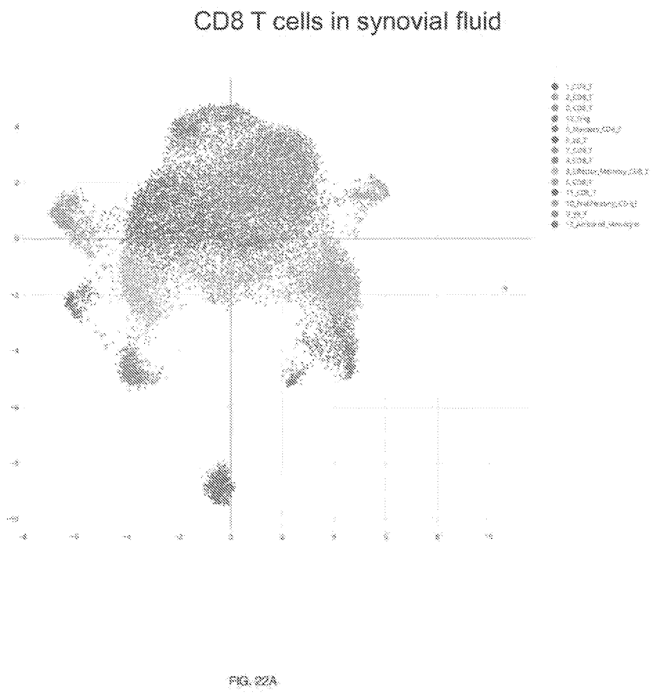

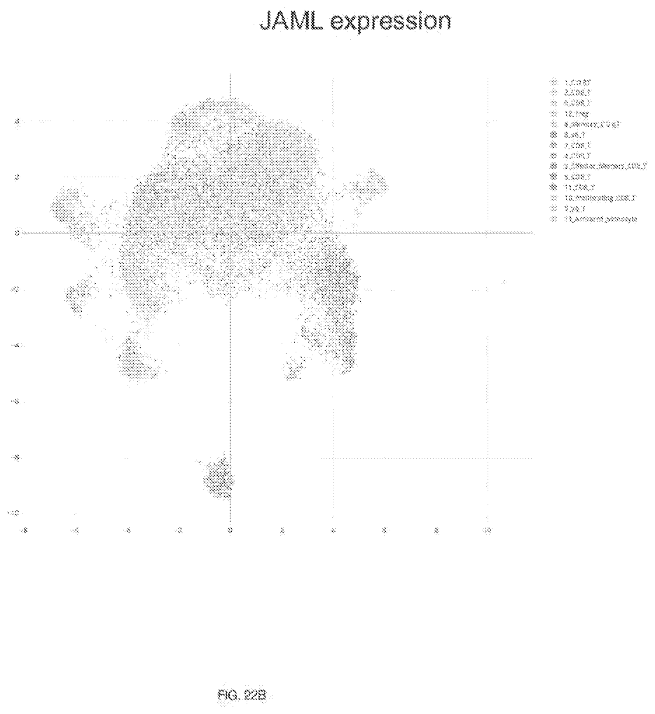

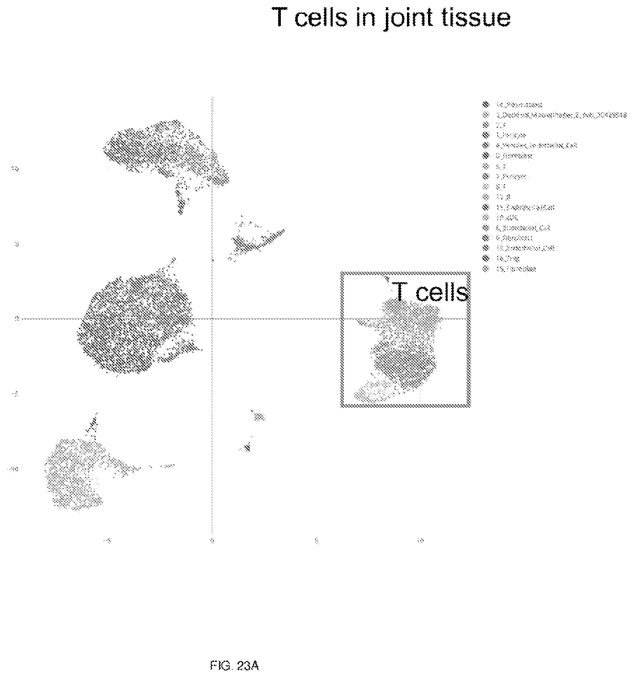

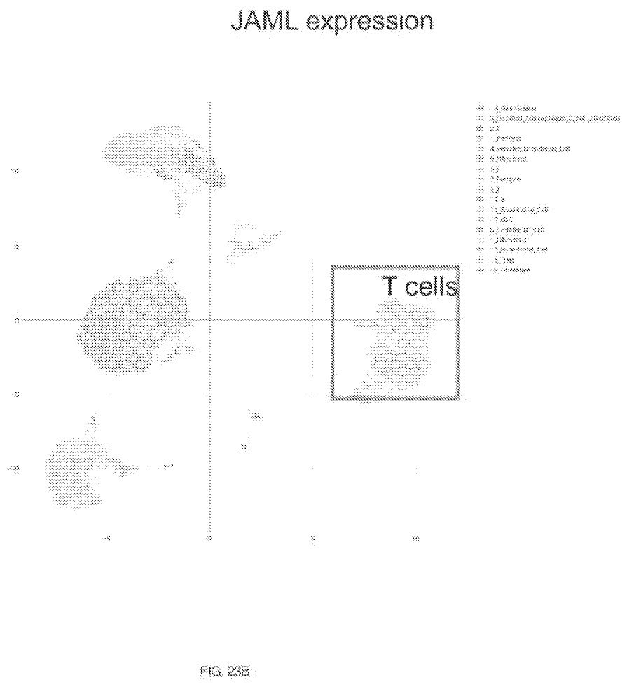

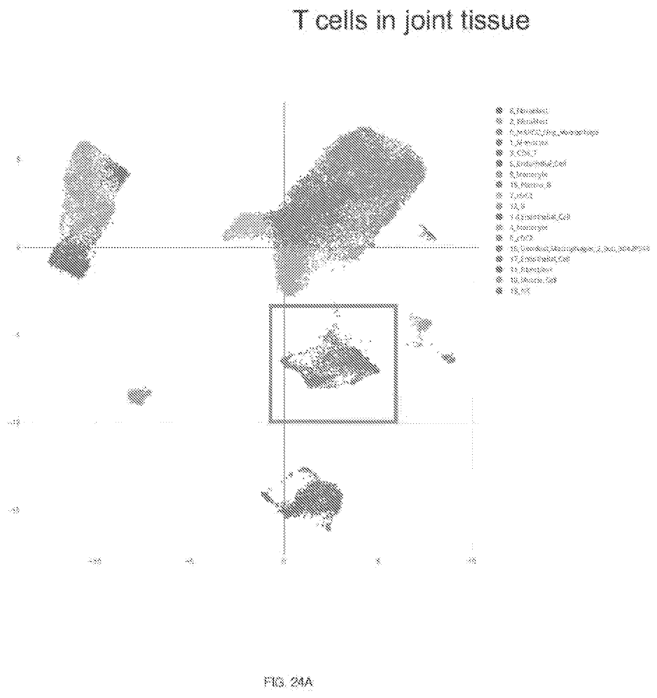

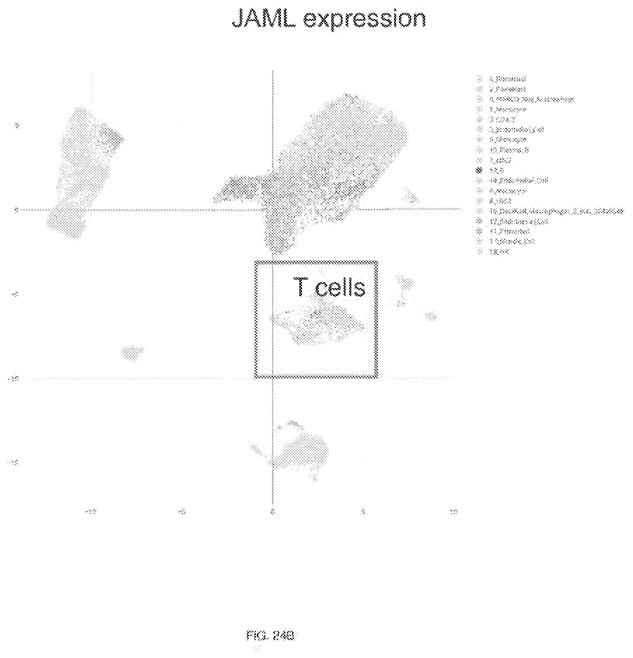

The inventors next validated the findings of single-cell transcriptome analysis by corroborating the proportion of CD4⁺ T cell subsets obtained by flow cytometric analysis of BAL cells from the same subjects. Based on the expression of cell surface markers, the inventors classified airway CD4⁺ T cells into 5 subsets: two types of T_{RM} cells (CD69⁺CD103⁻, CD69⁺CD103⁺), non-T_{RM} cells (CD69⁻), follicular helper T cells (T_{FH}, CXCR5⁺GITR⁻), and regulatory T cells (T_{REG}, CXCR5⁻CD127⁺CD25⁺) (FIG. 8A-D). As expected, the inventors found that proportions of CD103⁺ T_{RM} cells were significantly increased in males with severe asthma (50%) and positively correlated with the asthma severity score (FIGS. 2D and E). Together, these findings establish an important association between the numbers of CD103⁺ T_{RM} cells and asthma severity in male patients.

### Example 3: CD103⁺ T_{RM} Subset Displays Features Linked to Cytotoxicity and TCR Activation

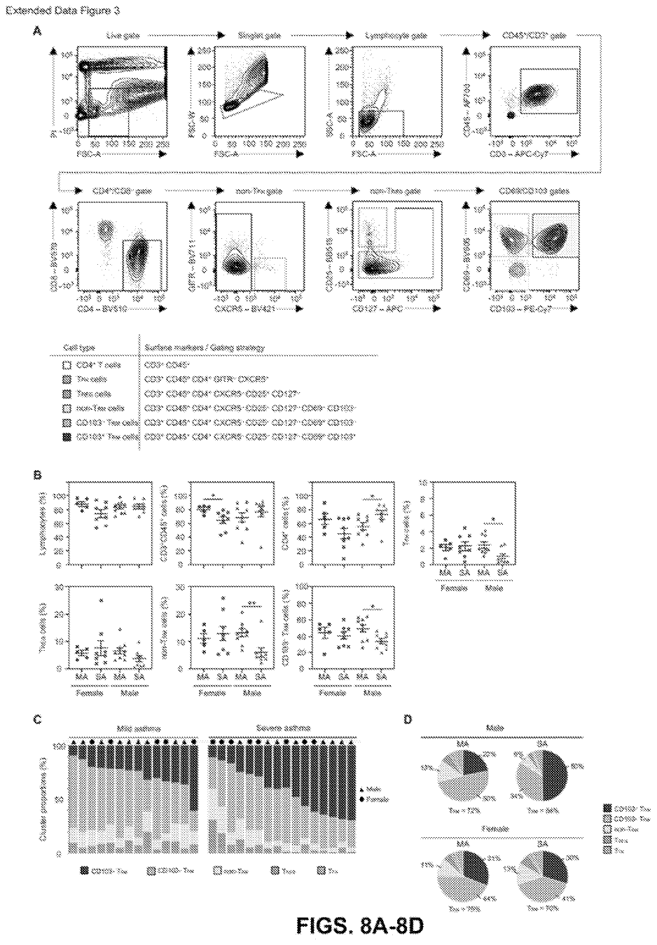

To determine the molecular properties of cells in the CD103⁺ T_{RM} subset that may contribute to the pathogenesis of asthma severity, the inventors first compared expression profiles of cells from the CD103⁺ T_{RM} subset (cluster 1) with those from the CD103⁻ T_{RM} subset (cluster 2), which was reciprocally reduced in males with severe asthma. The inventors observed major transcriptional differences between the two T_{RM} subsets with over 1,300 differentially expressed transcripts (FIG. 3A). Several genes involved in cytotoxic function (GZMB, GZMA, GZMH, FASLG)(46) were increased in expression in the CD103⁺ T_{RM} subset (FIGS. 3A and B,).

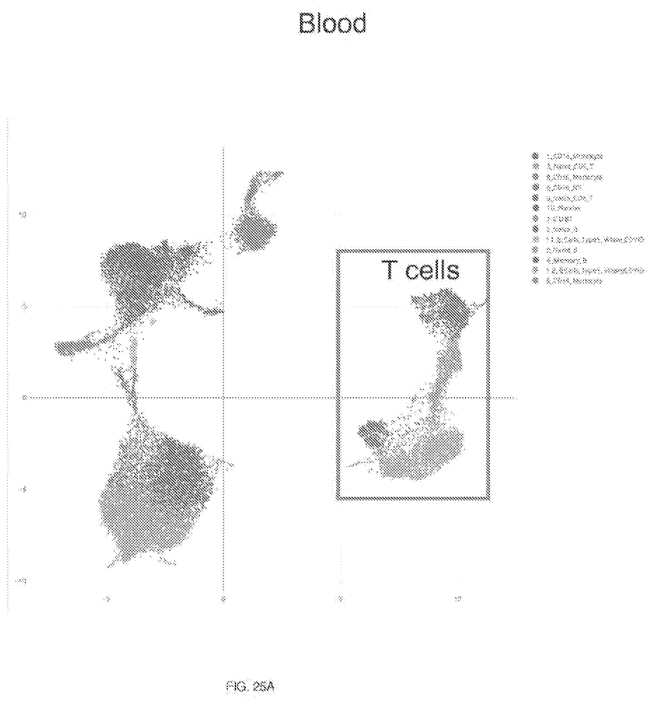

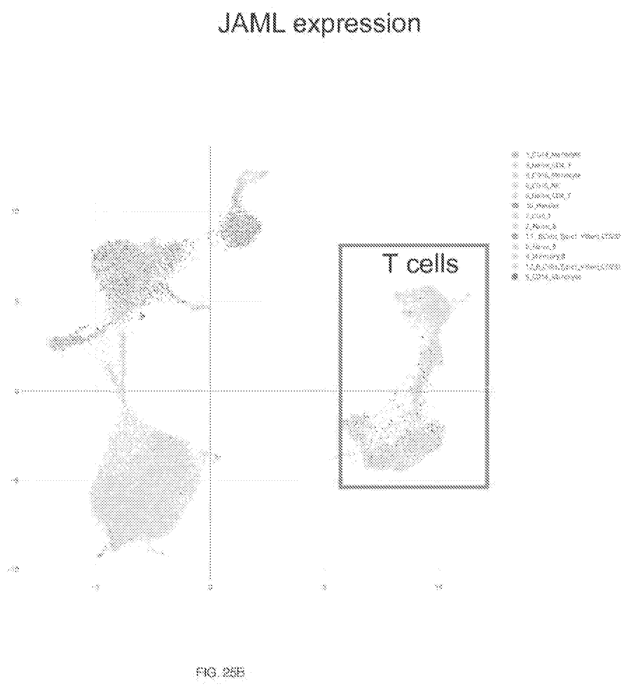

Gene set enrichment analysis (GSEA) and Ingenuity pathway analysis (IPA) confirmed significant positive enrichment of cytotoxicity signature genes in the CD103⁺ T_{RM} subset (FIGS. 3C and D), which indicated that cytotoxic CD4⁺ T cells are enriched in the CD103⁺ T_{RM} subset. The CD103⁺ T_{RM} subset also showed increased expression of transcripts encoding for two transcription factors linked to cytotoxic function in T cells: HOBIT (ZNF683), which is linked to T_{RM} differentiation and persistence of cytotoxic effector T cells(34, 50, 51), and HOPX, known to regulate GZMB expression(52) and to increase in vivo persistence of T_{H}1 cells, by reducing sensitivity to FAS-mediated apoptosis of T cells(53, 54) (FIG. 3B). Together, these findings suggest that the CD103⁺ T_{RM} subset is enriched for cells with increased cytotoxicity and effector properties, potentially driving airway inflammation and remodeling in severe asthma. Of note, bulk transcriptomic analysis of sorted CD103⁺ T_{RM} cells confirmed the increased expression of transcripts encoding for cytotoxicity-associated molecules like GZMB and the transcription factor HOBIT (FIGS. 9A and B).

IPA analysis of transcripts with increased expression in the CD103⁺ T_{RM} subset showed significant enrichment of genes involved in T cell receptor (TCR) signaling, CD28 and ICOS co-stimulation pathways (NFATC2, CSK, LAT, LCP2, CD40LG, CD52, HLA-DRB1, HLA-DRB5, HLA-DRA, MIR155HG), and survival pathways (ERK/MAPK signaling) (FIGS. 3E and G,). GSEA confirmed positive enrichment of genes involved in the TCR signaling pathway (FIG. 3F). Given that HLA-DR expression has been reported to mark recently activated T cells (55, 56), its increased expression in CD103⁺ T_{RM} subset points to an antigen-specific T cell activation in vivo (FIG. 3G). Transcripts encoding for several cytokines (IFN-γ, TNF, LIGHT) and chemokines (CCL4, CCL5), known to be involved in promoting airway inflammation and remodeling(57-59), showed significantly increased expression in the CD103⁺ T_{RM} subset (FIG. 3G). Notably, despite treatment with high-dose corticosteroid and/or biological agents, the sustained expression of transcripts linked to TCR activation and cytokines in CD103⁺ T_{RM} subset (FIG. 3G), suggests that treatment fails to curtail the activation and functional responses of airway CD103⁺ T_{RM} cells in severe asthma.

### Example 4: Molecules that Restrain T Cell Activation and Effector Functions are Reduced in Severe Asthmatics

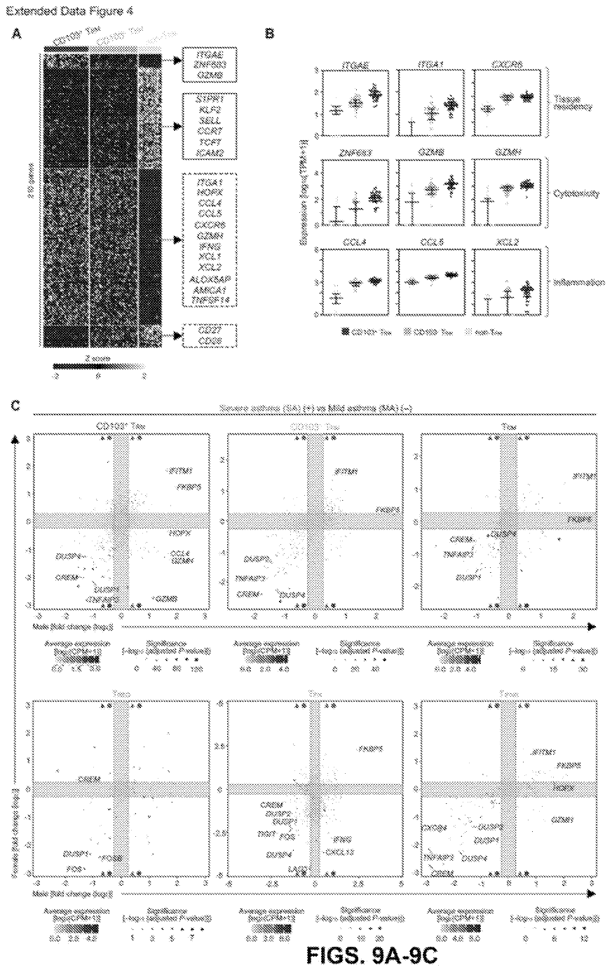

To determine qualitative differences in airway CD4⁺ T cell subsets that are associated with asthma severity in both males and females, the inventors performed independent single-cell differential gene expression analysis (sc-DGEA) of cells from severe versus mild asthmatics for each sex (FIGS. 4A and 9C). In both males and females with severe asthma, most CD4⁺ T cell subsets, including the T_{RM} subsets, showed significant reduction in the expression of several transcripts encoding for molecules (CREM, DUSP1, DUSP2, DUSP4, TNFAIP3) that are known to dampen TCR signaling and effector functions in T cells(60-64) (FIG. 4B). A notable example is cyclic AMP responsive element modulatory (CREM), a transcriptional factor that has been shown to repress promoters of inflammatory cytokine genes like IL2, IL13, IL4 and dampen type 2 inflammation in murine asthma models(62). As expected, GSEA confirmed negative enrichment of genes in the immunoregulatory cAMP signaling pathway(65) in cells from severe asthmatics (FIG. 4C). Other transcripts with reduced expression in severe asthma encode for several members of the dual specificity phosphatase (DUSP) family proteins: DUSP1, a glucocorticoid responsive gene product that is known to inhibit activity of mitogen-activated protein kinases (MAPKs) that trigger cytokine production in immune cells(60, 66-68), DUSP2 catalyzes dephosphorylation of STAT3 and inhibits T_{H}17 differentiation and inflammation(60, 69), whereas DUSP4 dephosphorylates STAT5 and negatively regulates IL-2 signaling and T cell proliferation(60, 70). TNFAIP3 is known to negatively regulate NF□B signaling that is involved in T cell activation and effector function(71, 72). In severe asthmatics, cells in the T_{FH} cluster also showed reduced expression of transcripts encoding for molecules linked to inhibitory function like PD-1, TIM-3, LAG-3, and TIGIT, which suggested the potential for unrestrained activity of airway T_{FH} cells in severe asthma(73) (FIG. 4D). T_{REG} cells from severe asthmatics had significantly reduced levels of transcripts encoding for AP-1 family of transcription factors (JUNB, JUN, FOS, FOSB) that have been shown to be important for its suppressive functions(74, 75) (FIG. 4D).

One of the most significantly increased transcripts in many clusters in severe asthmatics, in both males and females, was FKBP5, which encodes for the FK506 binding protein 5 (FKBP5), an immunophilin involved in the activation of NFAT/NFκB-driven pro-inflammatory signaling molecules(76-78) by inhibiting nuclear translocation of glucocorticoid receptor (GR), and thus mediating resistance to glucocorticoids (GC) (FIG. 4B). Overall, the sc-DGEA highlights the molecular properties of airway CD4⁺ T cell subsets that can potentially trigger their unrestrained activation as well as confer resistance to corticosteroids in severe asthmatics.

### Example 5: Non-T_{H}2 Pro-Inflammatory Cytokines are Expressed by Airway CD4⁺ T Cells from Severe Asthmatics

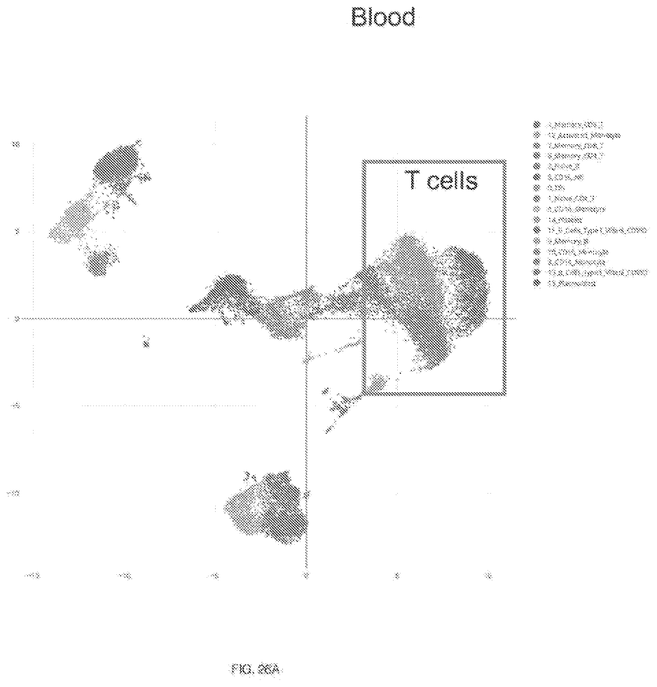

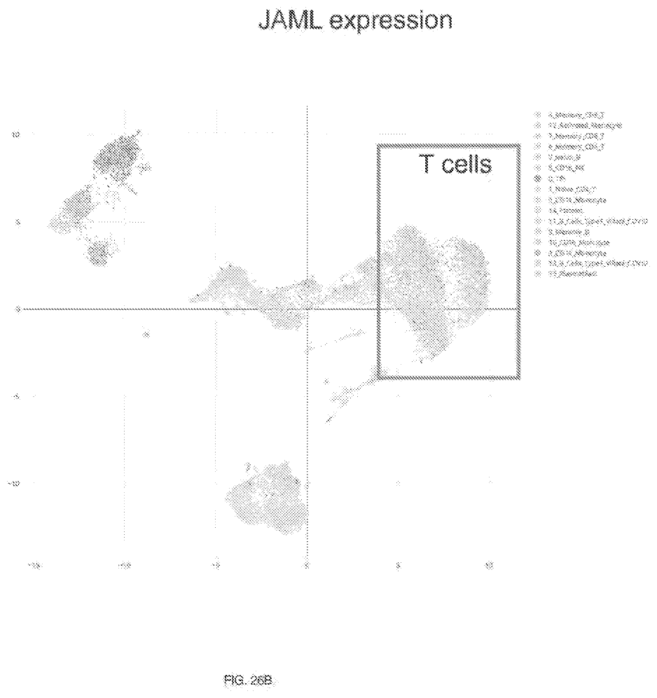

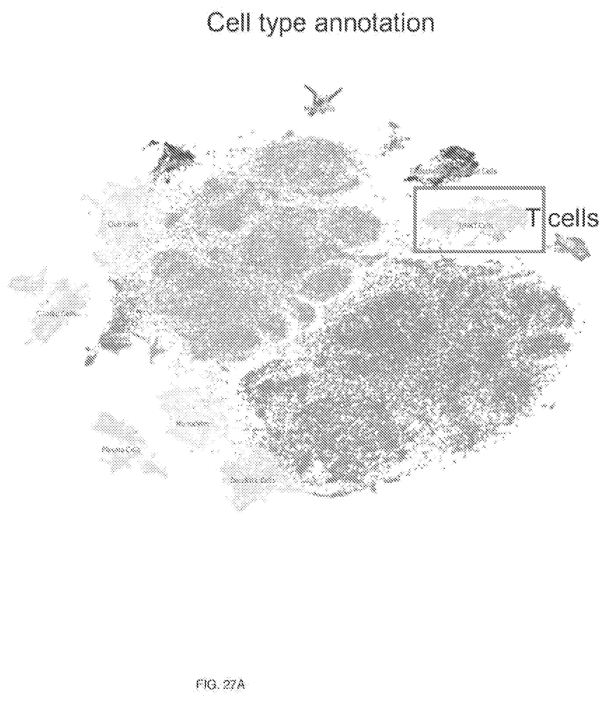

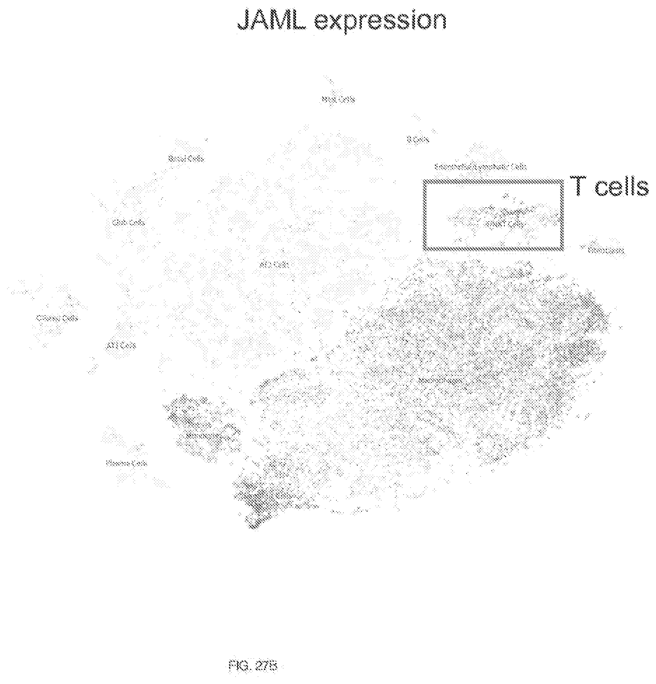

To examine the effector potential of CD4⁺ T cells present in the airways of patients with mild and severe asthma, the inventors stimulated, ex vivo, a fraction of BAL cells with phorbol 12-myristate 13-acetate (PMA) and Ionomycin for 2 hours, and performed single-cell RNA-sequencing on over 35,000 sorted CD4⁺ T cells (FIG. 5A, FIG. 10A-C). scDGEA of resting and stimulated CD4⁺ T cells as well as between stimulated cells from mild and severe asthmatic patients revealed several hundred transcripts linked to T cell effector function (FIG. 10D). As expected, transcripts encoding for chemokines known to be released by cytotoxic CD4⁺ T cells like CCL3, CCL4, and CCL5 (79-82) showed increased expression in stimulated airway CD4⁺ T cells from patients with severe asthma compared to mild asthma (FIG. 5A and FIG. 10E). Notably, a large fraction of airway CD4⁺ T cells expressed these chemokines at high levels in response to stimulation (FIG. 5A and FIG. 10E). These chemokines play key roles in the recruitment of several immune cell types expressing C—C chemokine receptor 1(CCR1), CCR3, CCR5, like neutrophils, monocytes, macrophages, NK cells, and T cell subsets(83), which have the potential to drive airway inflammation and remodeling(84, 85).

To further explore the effector molecules expressed by cytotoxic CD4⁺ T cells present in the airways, the inventors examined the co-expression of cytokine transcripts specifically in GZMB-expressing cells. The inventors observed high expression of transcripts encoding for several pro-inflammatory cytokines and chemokines such as TNF, IFN-γ, IL-17A, IL-21, IL-13, CCL3, CCL4, and LIGHT, which are all known to contribute to airway inflammation, fibrosis, and remodeling; in part through regulating activity of fibroblasts and smooth muscle cells(34, 58, 59, 86-95) (FIGS. 5B and C). These data suggest that cytotoxic CD4⁺ T_{RM} cells, besides their potential for direct killing of target cells, express pro-inflammatory molecules, thus they are likely to be critical players in sustaining airways inflammation and remodeling.

Although T_{H}2 cytokine transcripts were observed in stimulated airway CD4⁺ T cells, only a relatively small fraction of cells (1%, 2%, and 11%) were expressing IL5, IL4, and IL13 transcripts in patients with severe asthma respectively (FIG. 5A). However, several other pro-inflammatory non-T_{H}2 cytokine transcripts were expressed by a large fraction of airway CD4⁺ T cells (TNF (82%), CSF2 (39%), IL21 (32%), IL17A (7%) (FIG. 5A and FIG. 10E). Together, these data suggest that the effector potential of airway CD4⁺ T cells are not fully curtailed by high-dose corticosteroid treatment and that non-T_{H}2 cytokines may contribute to the pathogenesis of severe asthma. To confirm the effector potential of airway T_{RM} subsets, the inventors isolated specific CD4⁺ T cell populations from ex vivo stimulated BAL cells and examined their bulk RNA-seq profile. As expected, stimulated cells from the CD103⁺ T_{RM} subset expressed high levels of transcripts encoding for cytotoxicity-associated molecules (GZMB, GZMA, GZMH), pro-inflammatory chemokines (CCL3, CCL4, CCL5), and cytokines (TNF, IFN-γ, CSF-2, IL-21, IL-17A, IL-23A, IL-2, IL-13, LIGHT) (FIGS. 10F and G). Overall, these findings supported the existence of a population of T_{RM} cells with features of cytotoxicity and polyfunctionality in BAL samples collected from severe asthmatics.

### Example 6

Here, the inventors report on the single-cell transcriptomes from purified CD4⁺ T cells isolated from the airways of patients with severe and mild asthma. This unbiased approach led to the discovery of a cytotoxic CD4⁺ T_{RM} subset in severe asthma that the inventors hypothesize is critical in driving airway inflammation and remodeling in a specific subgroup i.e., males with severe asthma, where the inventors found a striking increase in the proportions of a CD4⁺ T_{RM} subset (CD103⁺ T_{RM} cells) with cytotoxic properties in the airways. Recent findings from the WATCH study demonstrated distinct clinical phenotypes of severe asthma stratified by age of asthma onset and sex(47). One previously poorly recognized phenotype was an adult-onset male phenotype that showed numerous adverse clinical features including worse lung function despite short disease duration, higher peripheral blood eosinophilia, higher exhaled nitric oxide and greater oral corticosteroid dependency. A further report from the WATCH study has characterized these adult-onset male severe asthmatics as being the subgroup that received anti-IL5 biologic drug, mepolizumab(96). Notably, this subgroup of asthmatics showed little of the typical psychophysiological comorbidities seen in difficult-to-treat asthma. This clinical phenotype has been previously shown in other studies though attracted little focus hitherto(97-100). In the present study, the inventors identified potential endotypic features i.e., cytotoxic CD4⁺ T_{RM} subset, that underpin such clinical phenotypes. The cytotoxic CD4⁺ T_{RM} cells displayed a unique pro-inflammatory cytokine and chemokine signature, highly enriched in transcripts encoding molecules (e.g. Granzymes, CCL3, CCL4, LIGHT, TNF, IL-21, IL-17A) that drive inflammation, cell death, and fibrosis. Because TRM cells are a long-term resident population in the airways, they have the potential for sustained interaction with airway structural cells and thus the products they release are likely to promote persistent airway inflammation and remodeling in severe asthma.

While T_{H}2 cells and to a lesser extent T_{H}17 and T_{H}1 cells have been implicated in asthma pathogenesis, and therapies targeting T_{H}2 cytokines are beneficial for some patients with asthma, the role of cytotoxic CD4⁺ T cells in severe asthma pathogenesis has not been previously described. Cytotoxic CD4⁺ T cell responses have been reported in certain viral infections such as human cytomegalovirus, HIV, dengue virus, hepatitis C virus, influenza virus, and more recently, with SARS-CoV2 virus(79, 101-111). Outside the context of viral infections, reports from studies employing single-cell genomics have shown an increased number of cytotoxic CD4⁺ T cells in patients with autoimmune diseases such as rheumatoid arthritis(112) and multiple sclerosis(113). These cells expressed high levels of pro-inflammatory cytokines and are hypothesized to drive disease pathogenesis. Most importantly, an increased number of cytotoxic CD4⁺ T cells has been observed in several steroid-resistant diseases with pronounced organ fibrosis such as systemic sclerosis(84), idiopathic pulmonary fibrosis(114), IgG4 disease(85), and graft versus host disease(115). It has been proposed that cytokines and other currently uncharacterized factors, released by cytotoxic CD4⁺ T_{RM} cells are likely to play a significant role in promoting fibrosis in the affected organs(85). Furthermore, an association between fibrosis and cytotoxic cell death of epithelial cells, endothelial cells, and fibroblasts in the affected organs has also been reported(84). Together, there is substantial precedent in other human diseases that cytotoxic CD4⁺ T cells can drive both inflammation and fibrosis in severe asthma through multiple mechanisms.

The inventors show that cytotoxic CD4⁺ T_{RM} cells in severe asthma express high levels of GZMA and GZMB transcripts. Notably, increased expression of Granzyme A and B has been reported in the airways of patients with fatal asthma(116). Granzyme A has been reported to cause pyroptosis (inflammatory cell death) of target cells expressing the protein GSDMB (gasdermin B) expressed by epithelial cells(117). GSDMB is one of the genes most linked to asthma susceptibility, and its expression has been shown to be increased in the airways of severe asthmatics(42). Therefore, it is highly plausible that Granzyme A released by cytotoxic CD4⁺ T_{RM} cells can cause pyroptosis of GSDMB-expressing airway epithelial cells in severe asthma.

In vitro studies of human airway epithelial cells have demonstrated that GSDMB in combination with Caspase 1 can induce epithelial cell pyroptosis(118). Furthermore, airway epithelial cells in response to interferons can express MHC-Class II and also present protein antigens to T cells(119). Thus, there is a possibility of direct cell-to-cell contact by MHC-II-antigen-TCR engagement with activated cytotoxic CD4⁺ T_{RM} cells directly delivering Granzyme A via perforin pores to cause GSDMB-dependent pyroptosis of epithelial cells, which can promote airway inflammation and remodeling events in severe asthma.

### Example 7: JAML Expression is Regulated by Interactions Between the CD3D and JAML Promoters

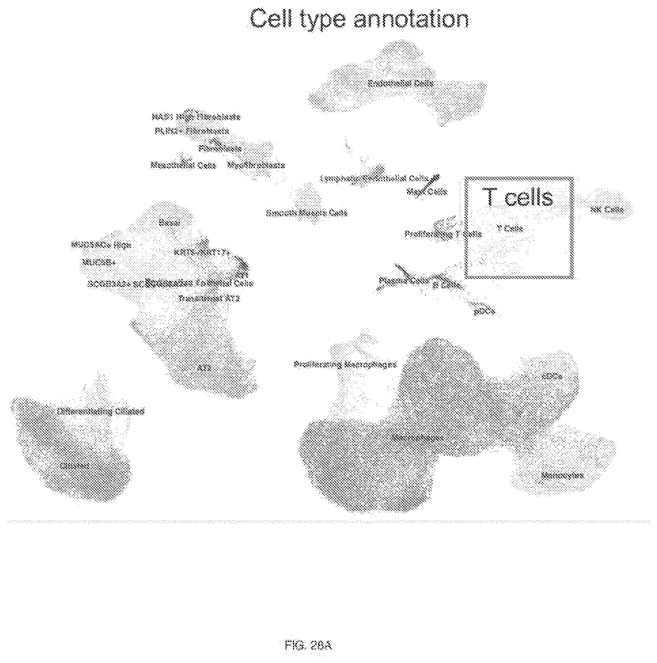

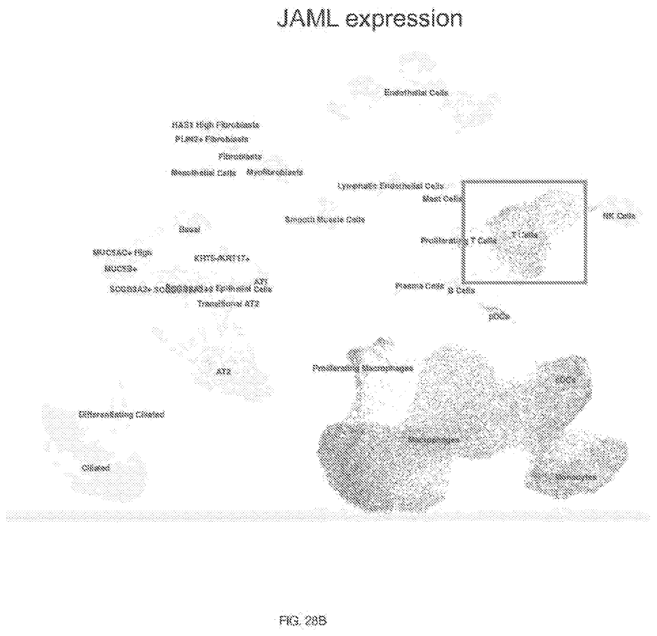

The inventors found that TCR stimulation more significantly increases JAML expression in human CD8+ T cells compared to CD4+ T cells (log₂ fold change 1.24 versus 0.37 in CD8+ and CD4⁺ T cells, respectively; FIG. 11A). To investigate how TCR signaling induces JAML expression in αβ T cells, the inventors first examined transposase accessible regions (ATAC-seq peaks) in the JAML locus in resting and stimulated human CD8+ and CD4+ T cells (FIGS. 11B 12A). Activation induced a strong ATAC-seq peak in the JAML intronic region (FIG. 11B) that also contained binding sites for NFAT, a key transcription factor involved in activation of genes following TCR activation. Notably, human tumor-infiltrating TRM cells displayed greater accessibility at the JAML promoter and the pertaining activation-induced intronic ATAC-seq peak region when compared to non-TRM cells. The applicant also found several NFAT binding sites in the promoter regions of upstream genes like CD3D and CD3G which encode for key components of the TCR, and which like JAML, showed increased expression following activation (FIG. 11B).

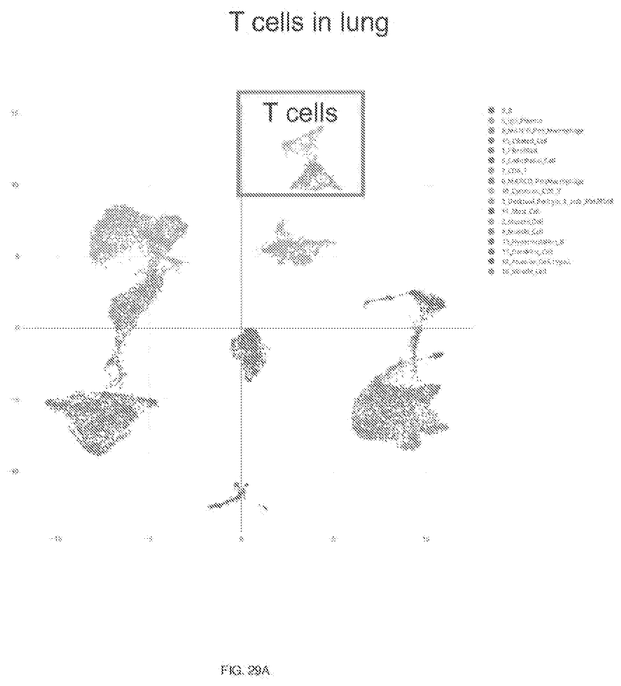

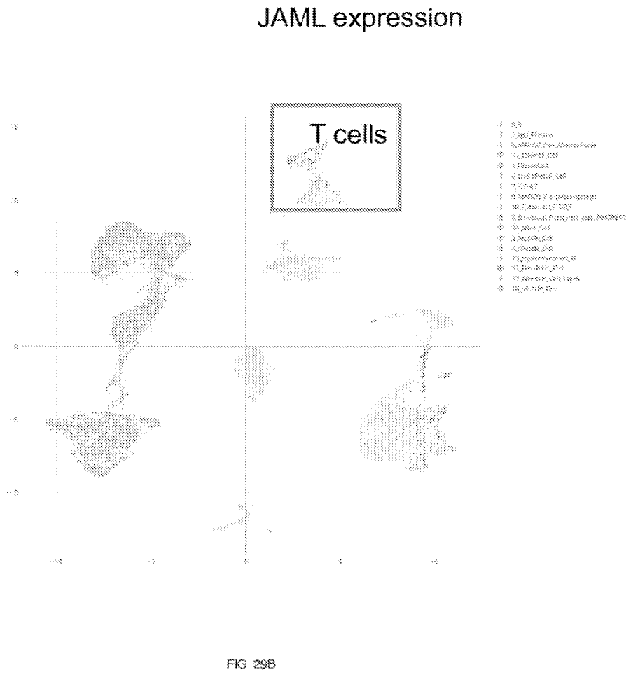

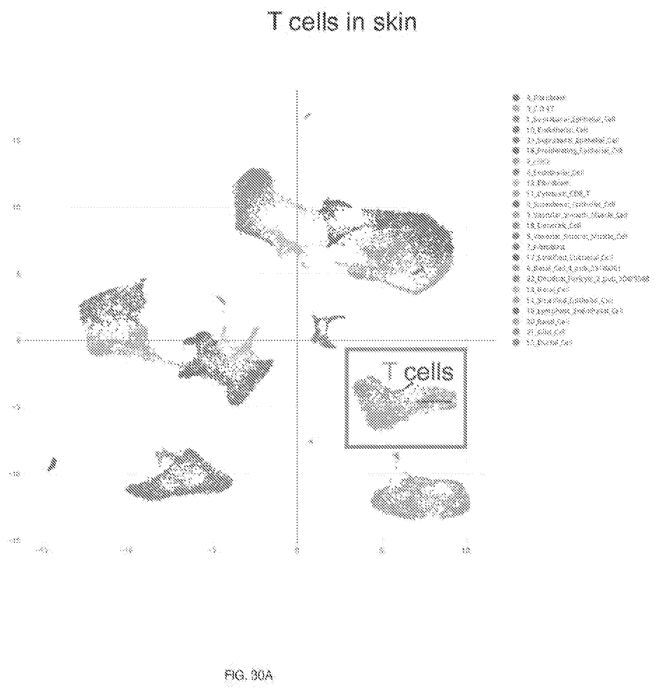

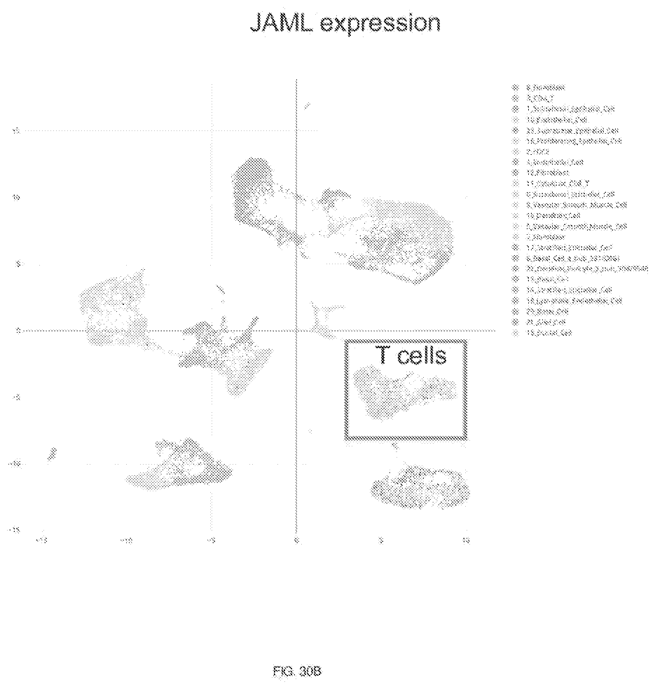

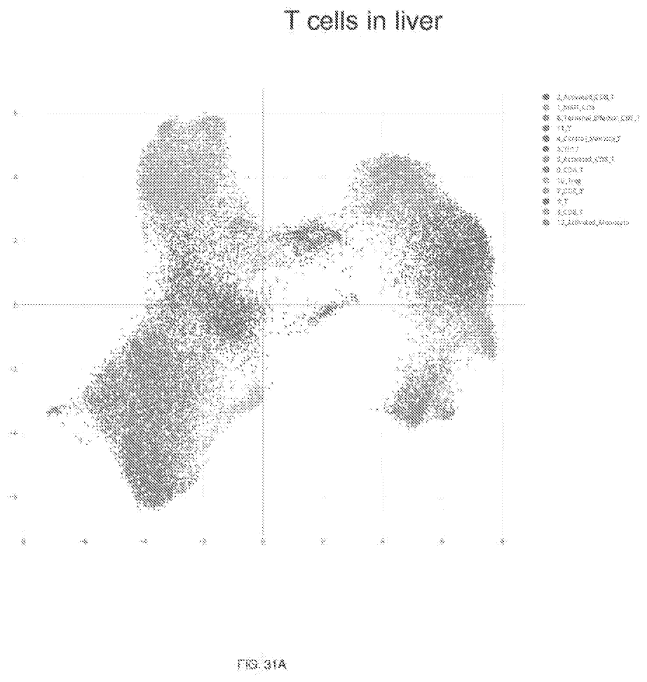

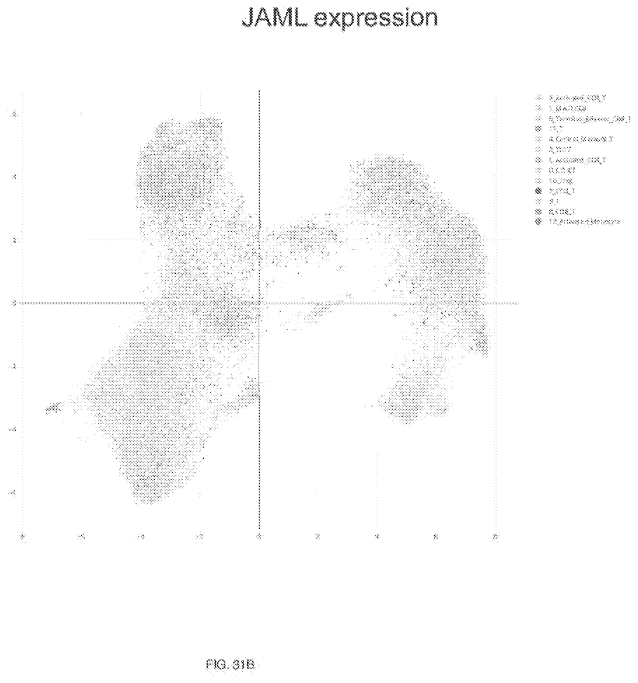

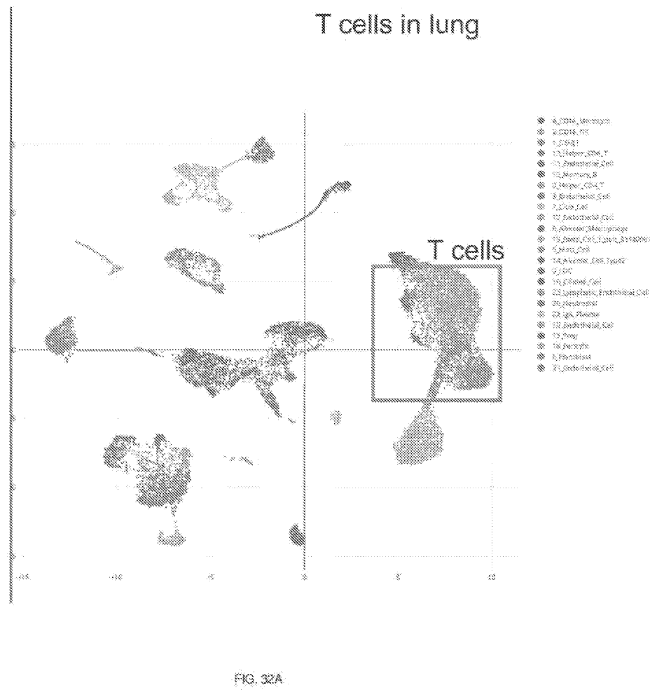

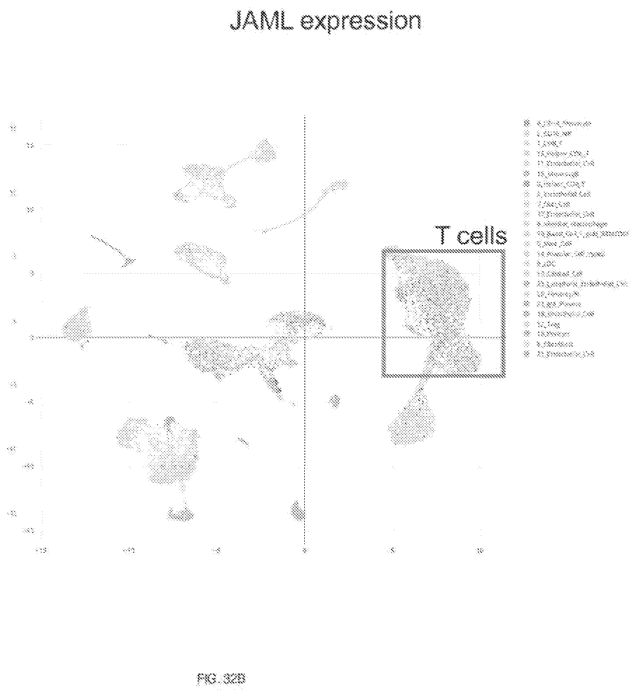

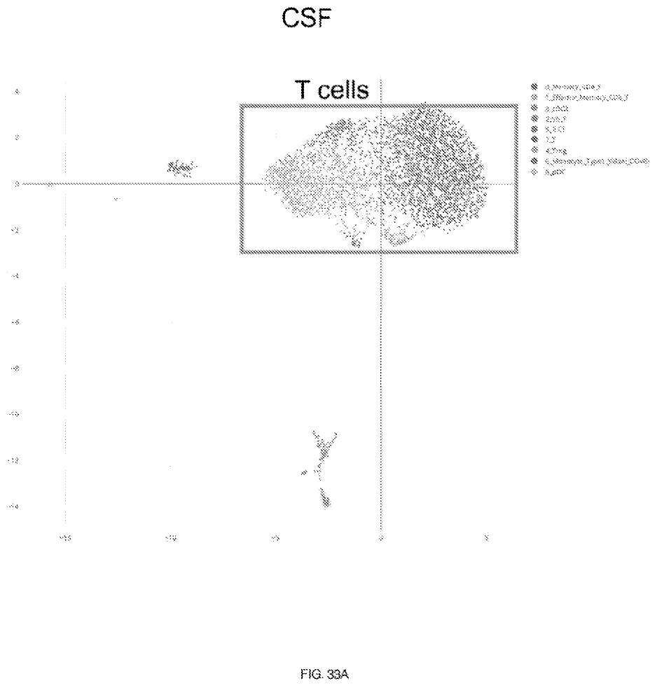

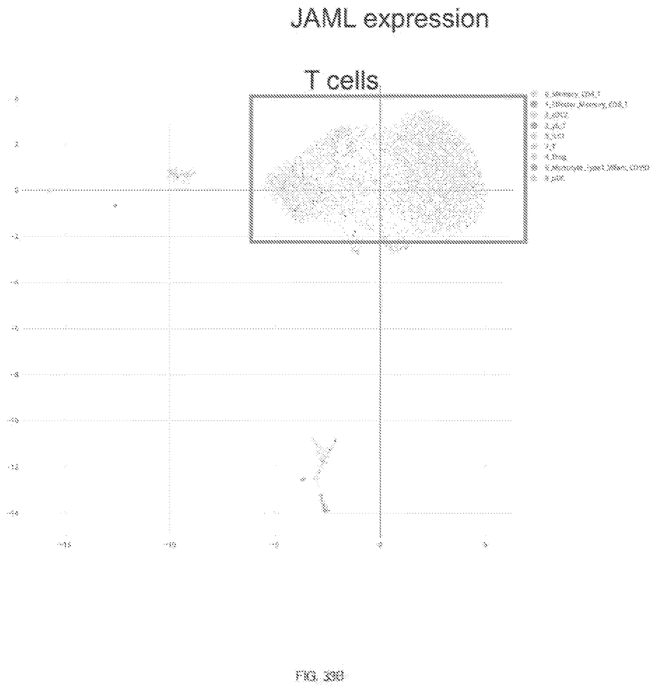

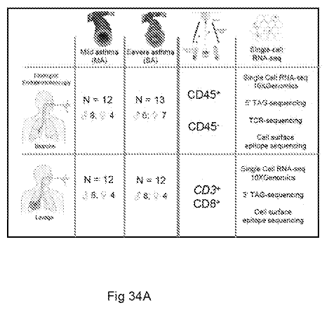

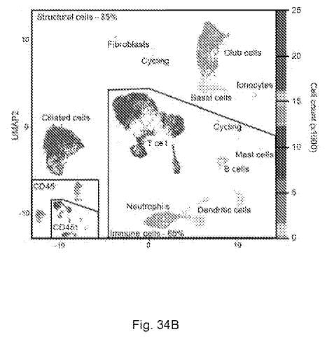

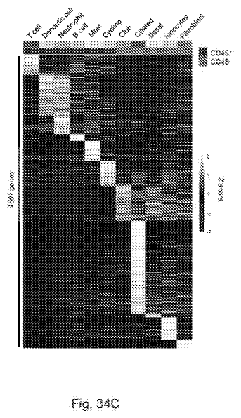

Importantly, by examining the 3D chromatin interaction map of the extended JAML locus in primary human T cells (Chandra et al., 2021), the applicant found that the JAML promoter and the activation-induced intronic cis-regulatory region strongly interacted with the neighboring CD3D promoter region (FIG. 11B), suggesting that they are likely to be involved in regulating JAML expression. Accordingly, the inventors found minimal interactions between these gene loci in other immune cell types (i.e., B cells or monocytes) that lack active CD3D promoter regions, indicative of a T cell-specific cis-regulatory control of JAML expression (FIGS. 11B and 12A). As the inventors have previously demonstrated that promoter-promoter interactions play a major role in regulating gene expression (Chandra et al., 2021), this data implies that the respective promoter regions of CD3D on the one hand, and JAML on the other hand, may act as reciprocal enhancers inducing each other's expression. TCR signaling is likely to increase NFAT binding and thus the transcriptional activity of the CD3D promoter, thus driving its own expression (FIG. 11C) and with it, the expression of JAML through long-range cis-regulatory interactions. Together, these data demonstrate how and why JAML expression is induced in human T cells by TCR engagement and implies a T cell-specific inducible expression profile of this co-stimulatory molecule. Crucially, these findings suggest that JAML expression is enriched in highly functional antigen-specific CD8+ TRM cells (i.e., reactive to tumor associated-antigens or neoantigens) driven by TCR-specific antigen-recognition and subsequent upregulation of JAML expression.

Table 1 demonstrates the involvement of JAML in autoimmune and fibrotic diseases, including asthma.

TABLE 1

| | DISEASE | | PMID or | | T cell | | Slide |
|---|---|---|---|---|---|---|---|
| ID | CATEGORY | DISEASE | GSE | number | expression | Tissue | no |
| 1 | ALLERGIC | Atopic | GSE147424 | 4 cases | JAML-T | Skin tissue | 1 |
| | DISEASE | dermatitis | | | cells | | |
| 2 | ALLERGIC | Eosinophilic | GSE201153 | 8 cases | JAML-T | esophagus | 2 |
| | DISEASE | esophagitis | | | cells | | |
| 3 | AUTOIMMUNE | Ulcerative | 31348891 | 18 UC | JAML | Colon | 3 |
| | DISEASES | Colitis | | and 12 | high | tissue | |
| | | | | controls | | | |
| 4 | AUTOIMMUNE | Crohn's | 31474370 | 11 cases | JAML | Ileum | 4 |
| | DISEASES | Disease | | | high | tissue | |
| 5 | AUTOIMMUNE | Psoriatic | 32958743 | 5 cases | JAML | Synovial | 5 |
| | DISEASES | arthritis | | | | tissue | |
| 6 | AUTOIMMUNE | Juvenile | GSE160097 | 7 cases | JAML- | Synovial | 6 |
| | DISEASES | arthritis | | | CD4 | fluid | |
| 7 | AUTOIMMUNE | Juvenile | GSE160097 | 7 cases | JAML- | Synovial | 7 |
| | DISEASES | arthritis | | | CD8 | fluid | |
| 8 | AUTOIMMUNE | Rheumatoid | GSE200815 | 4 cases | JAML- T | synovial | 8 |
| | DISEASES | arthritis | | | cells | tissue | |
| 9 | AUTOIMMUNE | Rheumatoid | E-MTAB- | 15 cases | JAML-T | synovial | 9 |
| | DISEASES | arthritis | 8322 | | cells | tissue | |
| 10 | AUTOIMMUNE | SLE | 142016; | 8 cases | JAML-T | blood | 10 |
| | DISEASES | | 162577; 16 | | cells | | |
| 11 | AUTOIMMUNE | Kawasaki | GSE167029 | 6 cases | JAML-T | blood | 11 |
| | DISEASES | disease | | | cells | | |
| 12 | FIBROTIC | Pulmonary | 30554520 | 8 cases | JAML | Lung tissue | 12 |
| | DISEASES | Fibrosis | | and 8 | | | |
| | | (IPF, ILD) | | controls | | | |
| 13 | FIBROTIC | Pulmonary | 32832598 | 20 cases | JAML | Lung tissue | 13 |
| | DISEASES | Fibrosis | | and 10 | | | |
| | | (IPF, ILD) | | controls | | | |
| 14 | FIBROTIC | Scleroderma- | GSE132771 | 4 cases | JAML | Lung tissue | 14 |
| | DISEASES | lung | | | | | |
| 15 | FIBROTIC | Scleroderma- | GSE160536 | 4 cases | JAML-T | Skin tissue | 15 |
| | DISEASES | skin | | | cells | | |
| 16 | FIBROTIC | Primary | E-MTAB- | 13 cases | JAML-T | liver tissue | 16 |
| | DISEASES | scleosing | 10143 | | cells | | |
| | | cholangitis | | | | | |
| 17 | LUNG DISEASE | COPD | GSE173896 | 5 cases | JAML-T | Lung tissue | 17 |
| | | | | | cells | | |
| 18 | BRAIN- | Alzeimer's | GSE134578 | 4 cases | JAML- | CSF | 18 |
| | IMMUNE | disease | | | Tcells | | |
| | DIESEASES | | | | | | |

### Example 8: CD8 TRM Cells are More Abundant in Severe Asthma Airways

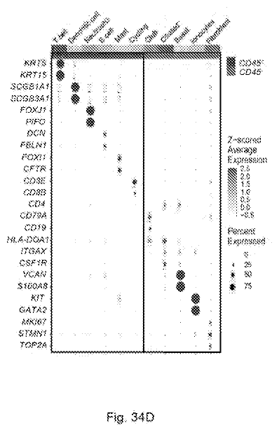

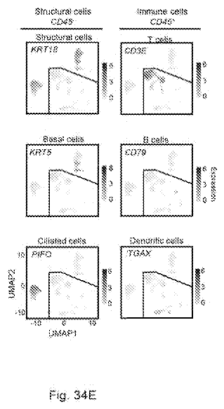

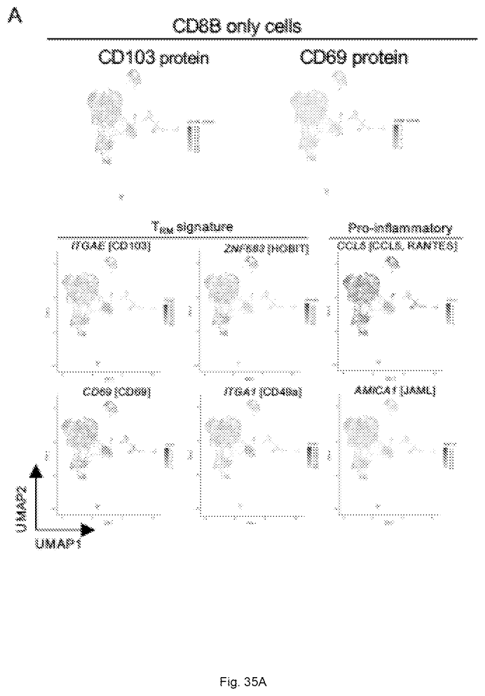

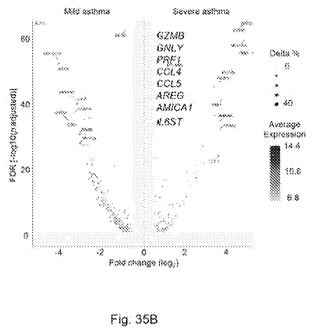

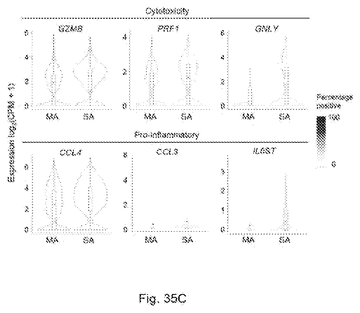

To unbiasedly identify quantitative and qualitative changes in the cellular and molecular compositions of the conducting airways and airway tissue in relation to asthma severity, the inventors enrolled and received consent to collect broncho-alveolar lavage (BAL) fluid as well as obtain endobronchial biopsies (biopsies), from 12 mild (66% males) and 13 severe asthmatic patients (100% Caucasians, 56% males, see methods; 152). As defined by the global initiative of asthma (GINA) management, severe asthma patients were currently on ‘high dose’ and/or ‘continuous or frequent use of oral corticosteroids’ (GINA step 4 and 5) 23,24 (FIG. 34A). All patients were in the same geographic area (Isle of White, UK) with a median age of 51, (IQR=37.25−61.5). For analysis, dispersed biopsy cells were thawed, and immune cells were separated from structural cells by a FACS cell-sorting strategy, based on the expression of the common hematopoietic cell surface marker, CD45 (FIG. 34). Briefly, the inventors sorted CD45+ cells (immune cell fraction) and CD45− cells (structural cell fraction) and performed single-cell RNA-seq, T cell receptor (TCR) sequence, and cell-surface protein epitope index-sequencing (see methods). Sequencing data went through a strict filtering process to enable identification of donor-specific single-cell transcriptomes, as well as eliminate doublets and low-quality libraries (see methods). With good quality single-cell transcriptomes from 12,743 CD45+ cells and 12,500 CD45− cells, an unbiased transcriptomic clustering analysis for all single-cell transcriptomes revealed a diverse repertoire of epithelial, stromal, and immune cell clusters (n=16) representing the lower airways of mild and severe asthma patients (FIG. 34B). Subsequent scDGEA analysis between clusters (Benjamini-Hochberg adjusted p-value<0.05 and log₂ fold change>0.25) identified 4,501 cluster-enriched transcripts, which the inventors used to define 11 biologically relevant cell subtype clusters (FIG. 34B-E). The CD45+ cells resident in the biopsy tissue predominantly consisted of T cells (70%—CD3E, CD8B, CD4), B cells (4%—CD79A, CD19, HLA-DQA1), dendritic cells (9%—HLA-DQA1, ITGAX, CSFIR), neutrophils (15%—VCAN, S100A8), mast cells (1%—KIT, GATA2), and cycling cells (1%—MKI87, STMN1, TOP2A) (FIG. 34B-E). The epithelial cells represented the CD45− compartment with defined cell types namely, basal (11%—KRT5, KRT15), ciliated (49%—FOXJ1, PIFO), and club cell (38%—SCGB1A1, SCGB3A1). The inventors also identified a cluster of fibroblasts (1%—DCN, FBLN1), and Ionocytes (1%—FOXI1, CFTR). The dominant T cell population identified by transcriptomic analysis were CD8 T cells, which were 53.6% of the sequenced fraction of CD45+sorted cells (FIG. 34B). This was confirmed by flow cytometry immunophenotyping, done prior to sequencing, showing approximately 60% of the sorted CD45+were expressing CD8B protein. No quantitative significant differences were observed when comparing both subsets between mild and severe asthmatics.

Table 1 demonstrates JAML involvement in autoimmune and fibrotic diseases, including asthma.

### Example 9: Severe Asthma Airway Biopsies are Enriched for CD8 TRM Cells with Enhanced Cytotoxicity, Pro-Inflammatory, and Tissue Repair Features

To capture the qualitative transcriptional differences in the dominant CD8+ T cells between both disease groups, CD8+ T cells were separated from the T cell fraction using the detection of CD8B cell-surface protein by single-cell index epitope sequencing (CITE-seq) (FIG. 35A). Interestingly, most of CD8+ T cells (approximately 97%±0.0128) expressed CD69 and CD103 protein molecules, two canonical surface markers defining tissue resident T cell population (TRM) (FIG. 35A). The inventors confirmed these results at RNA-level with observed corresponding expression for multiple TRM signature transcript genes (ITGAE [CD103], CD69, ITGA1 [CD49A], ZNF683 [HOBIT]). Interestingly, enrichment in transcripts coding for TRM activation (AMICA1 [JAML]) and inflammation (CCL5) in resting condition were found (FIG. 35A).

Analysis of the single cell transcriptome of all CITE-seq isolated CD8 T cells: 428 differentially expressed genes between mild and severe asthma patients were found (FIG. 35B). The inventors measured a significant upregulation of transcripts encoding for cytotoxicity (GZMB, GNLY, PRF1)25 and, proinflammatory chemokines (CCL4, CCL3) and cytokine receptor (IL6ST) in severe asthma patients compared to the mild patients was observed (FIGS. 35B, C). Interestingly, a transcript encoding for amphiregulin (AREG), a tissue repair and profibrotic cytokine, previously identified in T regulatory and TH2 cells (156) was significantly enriched in CD8+ T cells from severe asthmatics (FIG. 35D). The inventors analyzed the cytotoxic and profibrotic features in CD8+ T cells and found that the proportion of GZMB+AREG+co-expressing cells were significantly increased in severe disease (49.6%) compared to mild asthma (20.1%) (FIG. 35E). A high level of co-expression between cells expressing GZMB and other cytotoxic (PRF1, GNLY) or pro-inflammatory (CCL4, CCL5) transcripts (FIG. 36B) was found. In addition, high level of co-expression between cells expressing cytotoxic (GZMB, PRF1, GNLY) and pro-inflammatory (CCL4, CCL5) transcripts and AREG (FIG. 36C) were observed, as well as transcript coding for the TRM activation co-stimulatory molecule JAML, AMICA1, especially in severe asthmatics (FIG. 36F). Altogether, these data highlight enhanced polyfunctional effector capability (cytotoxic, pro-inflammatory and pro-fibrotic) of CD8 TRM response to tissue damage (FIG. 35G).

The inventors also observed a significant downregulation of TCR and TCR-signaling related molecule transcripts (CD247, ZAP70, CD81, METRNL) in severe asthma cells (FIG. 36D) potentially as a response to treatment with glucocorticoids as previously reported (157). This treatment response was confirmed by significant expression of gene transcripts linked to glucocorticoid resistance (FKBP528 and TSC22D329) in the severe asthmatics (FIG. 36D). Transcripts encoding for AP-1 signaling pathway associated molecules (JUNB, JUND) were significantly downregulated in severe patients, supporting the dampening of TCR signaling (FIG. 36D).

### Example 10: “Luminal” CD8+ TRM Cells in Severe Asthma Display More Cytotoxic, Inflammatory, and Innate Like Molecular Features

The inventors also investigated CD8+ T cells isolated from broncho-alveolar lavage samples (BAL), (referred to as luminal CD8+ TRM) collected from same patients to assess if the transcriptional phenotype observed from biopsy samples (mucosal CD8+ TRM) was shared and associated with the development of severe asthma disease (FIG. 34A). From BAL samples, CD8+ memory T cells contributed to about 38% of all BAL T cells and that 77% expressed markers of cell tissue residency (CD103 and CD69) indicating that, as observed in biopsies, most of the airway memory CD8+ T cells were TRM cells. The inventors obtained good quality single-cell transcriptomes for ≈9,599 BAL CD8+ memory T cells.

scDGEA comparing disease groups revealed 1,390 differentially expressed genes (FIG. 37A). Analysis of differential expressed genes in the severe asthma disease group (GSEA or pathway analysis) confirmed enrichment in transcripts involved in cytotoxicity (GNLY, GZMB, PRF1), pro-inflammatory molecules (CCL3, CCL4, CCL5), and co-stimulatory signaling molecule specific to TRM cells (AMICA1). Furthermore, upregulation of glucocorticoid (GC) responsive signaling genes linked to GC resistance (FKBP5, SAP30, DDIT4, IL6ST) (FIG. 37A-C) and cell survival (CFLAR, GIMAP1, GIMAP4) (FIG. 37A-C) were found. As described previously in the mucosal CD8 TRM, a down-regulation of the TCR engagement signaling transcript (ZAP70) as well as immunoregulatory molecules such as the NF□B regulatory molecule TNFAIP3 (TNFAIP3), the extrinsic cytokine-like molecule METRNL (METRNL) and the CAMP responsive element modulator CREM (CREM) (FIG. 37C) was found. Transcripts coding for innate stimulatory NK receptors molecules (KLRC2, KLRC4, KLRD1) were upregulated in severe asthma (FIG. 37C). The inventors confirmed the enrichment of pro-inflammatory molecules CCL3 and CCL4 at the protein level by Elisa measurement from BAL supernatants (FIG. 37D). Those data suggest the development of a CD8+ TRM subset with a more innate-like phenotype as previously reported in other immune disorders.

### Example 11: Disease Specific Heterogeneity in CD8 T Cells in BAL of Severe Asthmatics

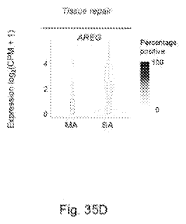

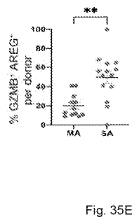

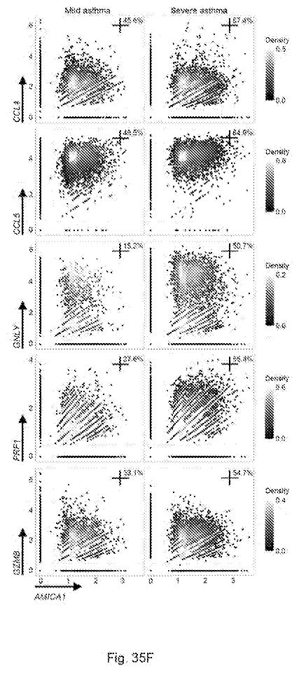

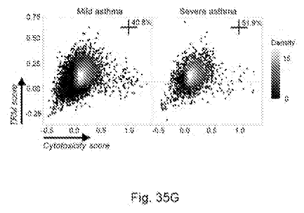

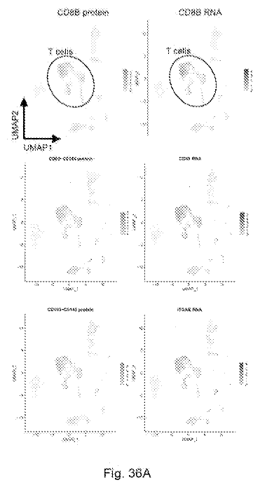

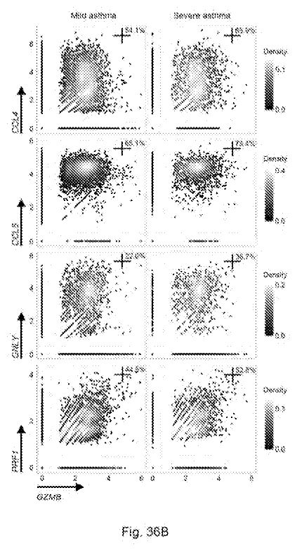

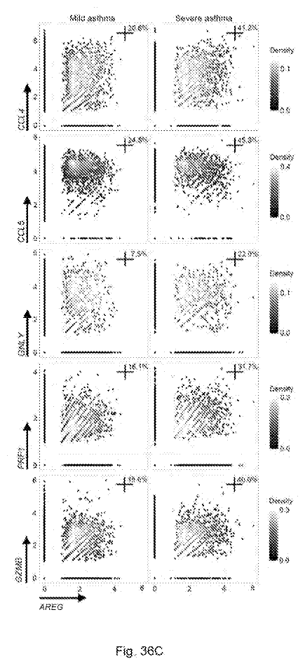

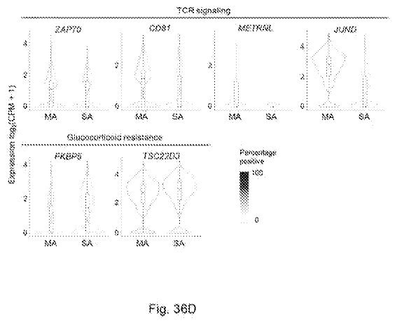

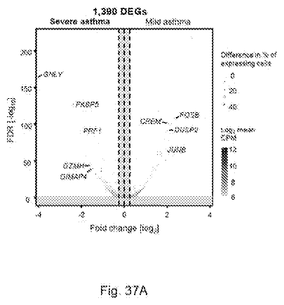

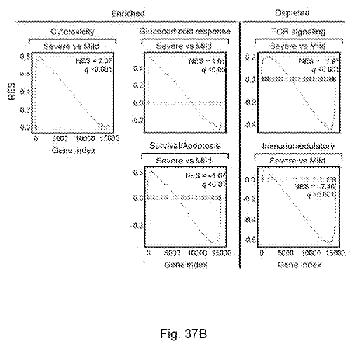

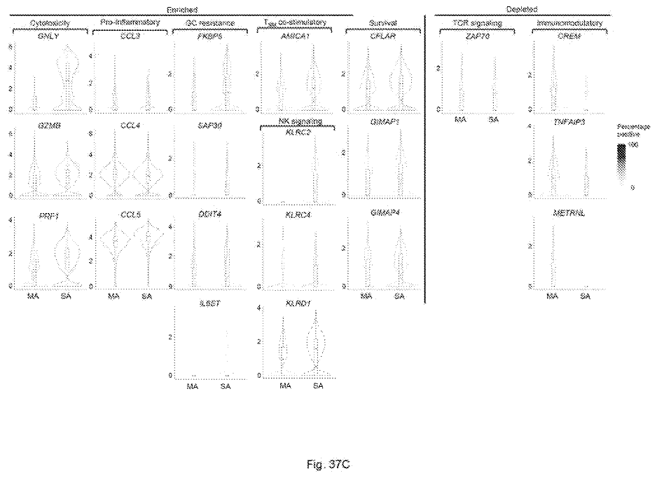

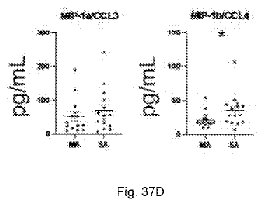

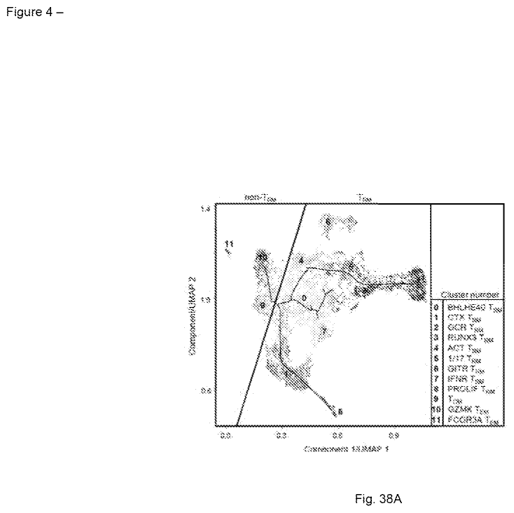

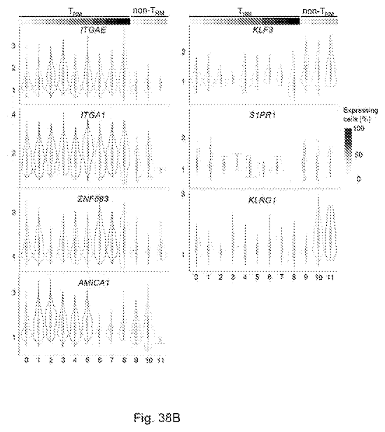

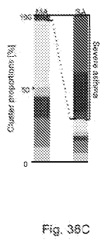

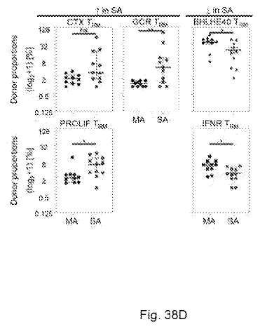

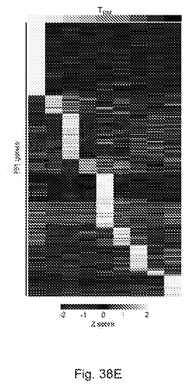

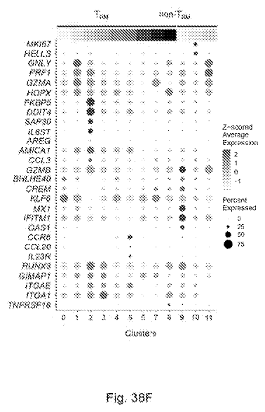

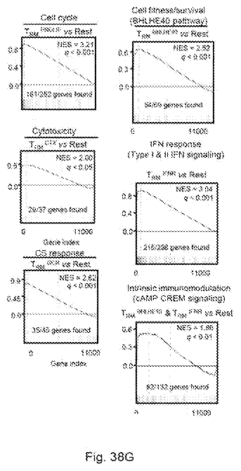

To evaluate if the disease specific transcriptional changes were measured in severe asthmatic were shared by all cells or only caused by a fraction of them, the inventors performed unbiased clustering analysis as well as differential gene expression analysis between clusters (see methods). 12 transcriptionally distinct clusters of CD8+ T cells were identified (FIG. 38A). Out of the twelve, 9 clusters (clusters 0-8) were highly enriched for TRM cell transcripts (ZNF683 [HOBIT], ITGA1 [CD49a], ITGAE [CD103], AMICA1 [JAML]). The remaining 3 clusters were enriched in non-TRM cell genes (i.e., KLF3, S1PR1, KLRG1) (FIG. 38B). Quantitatively, 3 TRM clusters (clusters 1, 2, 8) were exclusively observed in severe asthma and 2 (cluster 0, 7) were depleted in severe asthma patients (FIGS. 38C, D). Among the 3 TRM cell clusters that were exclusively observed in severe asthma, one cluster, the smallest (cluster 8) was composed of actively proliferating cells (MKI67, PCNA, MCM4, CENPK, and HELLS). The 2 larger clusters (cluster 1 and 2) were characterized by increased expression of cytotoxic transcripts (GZMA, GZMB, GNLY, PRF1). Cluster 2 also exhibited significant enrichment for glucocorticoid receptor response transcripts associated with GC resistance such as IL6ST, FKBP5, DDIT4 [Sharma et al., 2014]. Additionally, transcripts such as AMICA1 [JAML] and PDCD132 [Clarke et al 2019; Witherden et al 2010, Pardoll et al] linked to TRM activation and tissue retention, as well as natural-killer activating receptor molecules (KLRC2, KLRC4 and KLRD1) were also upregulated in those severe asthma specific clusters. Interestingly, AREG, the gene coding for the pro-fibrotic molecule Amphiregulin, was seen as Cluster 2 specific. These data indicate the development in severe asthmatics of a subset of CD8+ TRM cells with persistent activation, enhanced cytotoxicity, resistance to steroid treatment, and with an innate-like activating features.

Conversely, 2 TRM cell clusters (clusters 0 and 7), were significantly reduced in severe asthma (FIGS. 38F-G). These two TRM clusters (0, 7), mainly present in mild asthmatic patients, correlated positively with spirometry measurements of airway obstruction, and negatively with the frequency of acute asthma exacerbations (data not shown). The largest cluster (cluster 0) expressed transcripts linked to survival (BCL2), dendritic cell chemoattracting molecules (XCL1, XCL2) and TRM cell development, fitness, and enhanced functionality (BHLHE40). Notably, several transcripts reported to be under the transcriptomic regulation of BHLHE40 including i.e., ITGAE, KLF6, GZMB, IFNG, and CXCR6, are enriched within this cluster and other TRM clusters, supporting the role of BHLHE40 as a putative fate regulator for CD8+ TRM cells (FIGS. 38F-G) (163). The second cluster of cells depleted in severe asthmatics (cluster 7) was enriched for IFN response-associated transcripts (IFI6, IFI44L, MX1) with immune-regulatory transcripts such as TNFSF10 encoding for TRAIL, a trans-membrane molecule responsible for reducing TCR expression and therefore potentially dampening the TCR activation pathway of CD8+ T cells (164) (FIGS. 38F-G). Both Severe asthmatics depleted TRM cell clusters were enriched for transcripts associated with intrinsic and extrinsic immune modulation and/or inhibition such as the transcription factor cyclic AMP responsive element modulator (CREM), responsible for repressing gene transcription of IL2 and TH2 cytokines [Verjans Oncotarget 2015, Rauen 2013 Trends in Mol Med], thus being a negative regulator of TH2 responses and regulating inflammation, the SH3-containing immunoinhibitory adaptor SAM Domain, SH3 Domain And Nuclear Localization Signals 1 (SAMSN1 [Wang FASEB J 2010], and the anti-inflammatory molecule tumour necrosis factor-induced protein 3 (TNFAIP3) involved in suppressing NF-κB signaling. This data shows that the differential transcriptome of CD8 T cells in BAL of severe asthmatics was due to an impairment of possible CD8 intrinsic regulatory mechanisms shared in mild asthmatic patients.

Altogether, analysis CD8+ T cells, isolated from BAL and biopsies, provide qualitative molecular evidence that, in severe asthma, these cells a more proliferative, cytotoxic, and glucocorticoid resistant cellular state that may be driving the uncontrolled severe airway inflammation and poor response to corticosteroids observed in patients. Inversely, mild asthmatics CD8+ TRM displayed a more diver functionality linked to IFN response- and immunomodulatory-related cellular mechanisms indicative of a less pathogenic role.

### Example 12: Stimulated CD8+ TRM Confirm Pathogenic Status in Severe Asthma

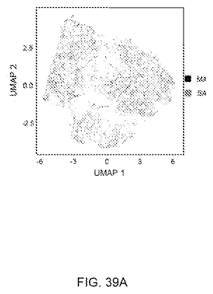

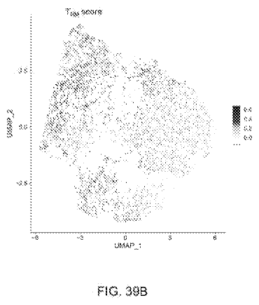

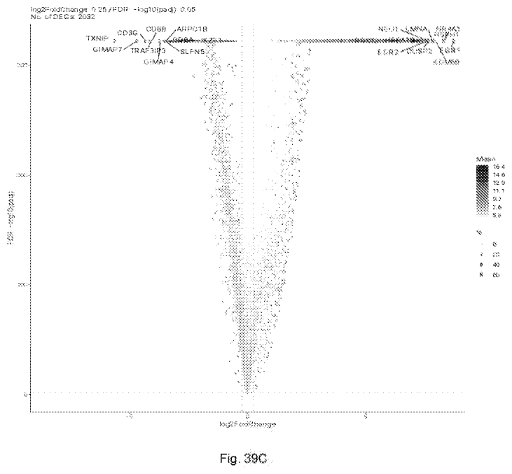

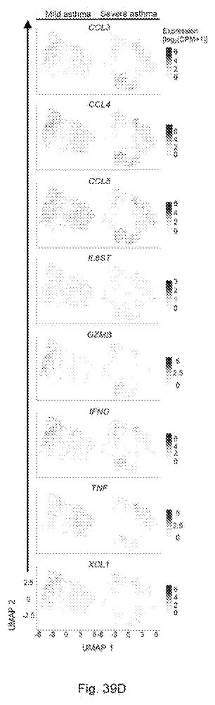

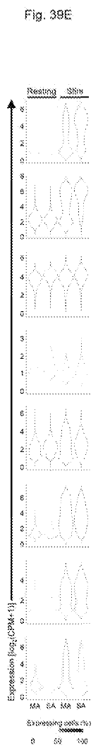

To gain insight into the molecular mechanisms of airway CD8+ T cells upon activation in severe asthma and determine the types of molecules induced by airway CD8+ TRM cells, the inventors also performed droplet-based single-cell RNA-seq on in vitro stimulated CD8+ T cells isolated from BAL with PMA and ionomycin for 2 hours (see Methods). 7,613 highly activated CD8+ T cells transcriptomes were analyzed. First, the inventors confirmed TRM cells were still the dominant CD8 subsets after stimulation. scDGEA between resting and stimulated CD8+ T cells revealed the up regulation of more than 1,000 genes (FIG. 39B). Gene set enrichment and pathways analysis identified transcripts involved in airway remodeling (TNFSF14 [LIGHT], TNFRSF12A [TWEAK receptor], CEBPB, CEBPZ), glucocorticoid sensitivity (DUSP4, NR3C1), and TCR dampening signaling (EGR1, EGR2, NR4A1) (FIG. 39C). The inventors then looked for differentially induced genes between disease groups (FIG. 39D) and found that transcripts up-regulated in severe asthma were coding for pro-inflammatory molecule such as chemokines (CCL3 ([MIP]-1α), CCL4 [MIP-1β], CCL5 [RANTES]), and their cognate C—C type chemokine receptors (CCR)1 and CCR5 (FIGS. 39C-D). These chemokines are critical for immune surveillance through the recruitment of myeloid cells (165). Furthermore, CCL5 [RANTES] is associated with airway pro-inflammatory functions (166-168). In addition, increased expression of interleukin (IL)-32 was observed; IL-32 is a pluripotent cytokine that can induce proinflammatory factors such as IL-1β, IL-6, IL-8, TNFα, and CCL4; and IL6ST (gp120) encoding for the common receptor subunit (gp130) for the inflammatory cytokines IL-6, IL-27, and IL-11 (FIGS. 39C-D). These cells were also highly enriched for transcripts linked to enhanced cytotoxic effector function (GZMB, GZMA, GNLY, PRF1) and stimulatory NK receptor signaling (KLRC2, KLRD1) 39; (iii) airway tissue damage and remodeling (CD63, GZMB, GZMA). Notably, with severe asthma patients having received much treatment (OCS and/or biologics therapy), upregulation of glucocorticoid resistance genes (FKBP5, CEBPD) (FIGS. 39C-D) was observed, suggesting that CD8+ T cells are not quiescent but potentially play a critical role in response to treatment and thus in the pathogenesis of severe asthma.

Conversely, activated CD8+ TRM cells from severe asthmatics exhibited significantly less transcripts for molecules such as other proinflammatory cytokines (IL2, IFNG, TNF, CSF2, IL23A), chemokines such as CCL3 and XCL1 and cognate receptors (IL2RB, IL27RA, IL4R, IL21R, IFNGR1, TNFRSF1B). They were also significantly enriched for transcripts involved in immune regulation (METRNL, CREM, TNFRSF18 [GITR] DUSP4, SAMSN1, SOC3), as well as anti-inflammatory mechanisms (FOS, TNFAIP3, TNFAIP8) consistent with the findings from resting cell states.

Overall, these analyses revealed that upon activation, CD8+ TRM cells from severe asthmatic patients were profoundly cytotoxic and displayed an unrestrained pro-inflammatory potential supporting a key role in driving the uncontrolled airway inflammation in the pathogenesis of severe asthma.

### Example 13

The inventors herein characterized, at single-cell resolution, the severe asthmatic lower airways mucosal and lining tissue compartments. Quantitative assessments revealed a significant enrichment of tissue resident CD8+ T cells (CD8+ TRM) with a strong positive association with clinical disease severity indices [Bratke et al., 2004; den Otter et al., 2016], presumably highlighting their important role in driving asthma severity.

This analysis confirms the existence of a highly cytotoxic and pro-inflammatory subset of CD8+ TRM cells as the dominant inflammatory cell state observed in both the luminal airway lining (BAL) and mucosal bronchial tissues of severe asthmatics. It was also found that this predominant CD8+ TRM phenotype in the airways of the asthmatics is positively associated with lung function decline and asthma severity scores. This data also revealed multiple novel cellular and molecular features associated to the CD8 T cell subset and tissue source. For instance, the inventors showed that ‘mucosal’ CD8+ TRM cells were clonally expanded and actively expanding in severe asthma patients (data not shown), despite receiving treatment with high dose corticosteroids and/or biological therapies predominantly targeted at TH2 immune responses. In absence of clear antigenic stimulation, it is unclear why these cells continuously undergo cell proliferation. Without being bound to a particular theory, these cells are reacting to environmental antigens such as allergens, latent lung trophic viruses (van de Berg, Yong, Remmerswaal, van Lier, & ten Berge, 2012)), or to self-derived protein generated from uptake and presentation by APCs of products of surrounding chronic tissue damage (activated autoreactive T cells via epitope spreading) (176, 177). The latter phenomenon has been observed in autoimmune diseases 48. Alternatively, the inventors also found that ‘luminal’ CD8+ TRM cells isolated from the airway luminal compartment (BAL) were enriched in activating NK-activating receptor molecules (KLRC2, KLRC4, KLRD1) potentially suggesting the adoption of innate cell properties leading to a less TCR-specific activation.

These data support an enhanced pro-inflammatory role for the CD8 T cells isolated from severe asthmatics. First, in resting state, high levels of expression of granulysin (GNLY) and granzymes (GZMA, GZMB) genes were observed, classically known for their cytotoxic role, but have also known as inducers of proinflammatory mediators. As an example, granulysin, a saposin-like pore-forming protein, can function as a potent chemoattractant and induce several inflammatory molecules in autoimmune diseases such as CCL2 (MCP-1), CCL5 (RANTES), TNFα, IL-6, IL-8, and IL-12 Deng et al., 2005 JI; Tewary et al., 2010 Blood). Similarly, granzymes A and B can induce the proinflammatory cytokines IL-6, IL-8, and IL-1β via proteolytic cleavage of their precursors (Afonina et al., 2011, Wensink et al., 2015), as well as cause degradation and cleavage of extracellular matrix proteins (e.g. fibronectin, vitronectin) to further potentiate inflammation, initiate extracellular matrix remodeling and fibrosis [Buzza et al., 2005 JBC, Shen et al., 2016 AmJPathol, Wensink et al., 2015]. Second, analysis of activated severe asthma CD8+ TRM cells, revealed high level of co-expression for transcripts encoding for proinflammatory chemokines (CCL3, CCL4, CCL5), cytolytic molecules (GZMB), cytokines (IL32), and cognate receptors (CCR5, CCR1, IL6ST) supporting the hypothesis on the development of a pathogenic polyfunctional CD8+ TRM cell state contributing to asthma pathogenesis and even asthma death (179, 180). Additionally, an associated aberrant immunoregulatory program observed in the severe asthmatics could likely be driving the enhanced cytotoxicity as TNFAIP3 deficient CD8 T cells have been shown to have augmented proinflammatory cytokine production, especially, increased GZMB production and thus improved clearance of bacterial infection (181) and anti-tumor activity (160). This augmented cytotoxic program is pathogenic and drives severity of disease.

One other clear molecular signature that was identified in the CD8+ TRM from severe asthmatics is the response and resistance to glucocorticoid treatment. For example, FKBP5 encoding the FKBP51 protein, a co-chaperone of the glucocorticoid receptor (GR) complex is a direct inhibitor of GR nuclear translocation, thus reducing GR-induced transcriptional activity [Reynolds et al., 1999, Scammell et al., 2001] confirming previous studies suggesting that elevated expression of FKBP5 confer glucocorticoid resistance in asthma and chronic obstructive pulmonary disease [Stechschulte & Sanchez 2011) Woodruff et al., 2007]. This significant finding provides molecular rationale for a fraction of severe asthmatic patients managed in clinics that are refractory to corticosteroid treatment and continue to suffer from persistent airway inflammation, asthma exacerbations and poor asthma control. However, FKBP5 expression is potently induced by corticosteroids and reports have shown its expression can affect clinical responsiveness to corticosteroid treatment (182, 183). Its upregulation has been associated with inhibition of NFκB regulatory mechanisms, therefore leading to unrestrained NFκB driven inflammation (159, 182), making it a potential driver of the enhanced inflammatory response in the severe patients particularly with the luminal CD8 T cells. A dexamethasone induced FKBP5 upregulation in CD4 T cells has recently been associated with worse control of asthma in obese children (184). Therefore, with overall increased expression in the both the airway CD8 T cells and the epithelial cells in severe patients compared to the steroid free asthma patients with controlled disease, this places FKBP5 as a suitable therapeutic target for treating steroid resistant asthma. Additionally, other GR upregulated transcripts linked to TRM activation, retention and persistence such a AMICA1 [Clarke et al., 2019, Witherden et al., 2010, PDCD1; Pardoll et al. 2012] as well as IL6ST were seen in severe asthma CD8 TRM cells. Particularly, IL6ST encodes the common subunit (gp130) of cognate receptors for the proinflammatory cytokines IL-6, IL-27 and IL-11, all known factors promoting the development of cytotoxic molecular pathways [Hilde, West 2019 fimmunol]

Finally, it is herein shown that the highly cytotoxic mucosal CD8+ TRM cells have a persistent production of tissue pro-fibrotic amphiregulin (coded by AREG gene) which is known to facilitate airway fibrosis and remodeling in chronic lung disease [Morimoto et al., 2018; D. M. W. Zaiss, Gause, Osborne, & Artis, 2015]. This is the first report of CD8 T cells showing a prominent remodeling program that might be detrimental to driving severity of asthmatic disease. Previously, amphiregulin was commonly known to be produced by CD4 T cells [D. M. Zaiss et al., 2006], and innate cells in the context of type 2 immune mediated responses [D. M. Zaiss et al., 2015]. The expression of AREG in CD8+ T cells, remained limited to tumor infiltrating cytotoxic T cells with clear correlation with tumor progression and the description in the lung airways reveals a potential driver of airway remodeling in severe asthmatic disease [Kwong et al. 2010]. Remodeling corresponding increased level of soluble amphiregulin has been detected from blood and sputum fluid samples of asthmatics and was associated with asthma severity, revealing a potential use as a biomarker for severe asthmatics and possible therapeutic target.

Although AREG expression has been prominently shown in chronic fibrotic diseases [Goplen et al., 2020; Habiel et al., 2019; Maehara et al., 2020; Mattoo et al., 2016] TGF-β has been cited as a commonly known regulator of AREG expression [Bennett, Plowman, Buckley, Skonier, & Purchio, 1992]. Interestingly, the expression of TGFB gene is down regulated in the tissue CD8+ TRM cells from severe asthmatics patients presumably contributing to the enhanced uncontrolled expression of AREG and its resultant remodeling capacity. Therefore, overall, this data shows an altered prominent cytotoxic and profibrotic CD8 TRM cell associated with severe asthmatic disease.

Defective epithelial barrier function, a cellular hallmark of severe asthmatics airways, has been associated with promoting sensitization to allergens and driving asthma pathogenesis [Heijink et al., 2020; Holgate, 2007]. Consequently, aberrant epithelial repair responses lead to airway remodeling which further exacerbates disease severity [Heijink et al., 2020; Holgate, 2007]. As this data shows an increased expression of AREG on mucosal CD8 T cells, epidermal growth factor receptor (EGFR) on proximal epithelial cells, as well as enrichment for airway remodeling and fibrosis related pathways, the CD8+ TRM AREG+ effector cell interaction with EGFR+ epithelial cells, AREG-EGFR signaling, may contribute to the increase in airway remodeling and fibrosis leading to asthma severity progression.

To summarize, the exploratory analysis, made from a unique set of cells collected from severe asthmatic patients, provide a clear school of evidence supporting a functional shift of CD8+ TRM cell states in severe asthma towards a highly pathogenic phenotype with molecular features linked to enhanced cytotoxicity, inflammation, glucocorticoid resistance and long-term TRM, non-specific, activation and persistence. The progressive increase of this highly clonally expanded cytotoxic effector CD8+ TRM cell with a prominent tissue remodeling molecule, amphiregulin, potentially drives the crucial fibrotic remodeling of epithelial tissue thus exacerbating disease. This aberrant cytotoxic and profibrotic program in the CD8+ tissue resident memory cells drive severity of asthmatic disease in treatment-unresponsive patients and therefore highlight the need for better targeted therapy.

### Materials and Methods

### Subject Recruitment, Ethical Approval, and Characteristics

Subjects diagnosed with asthma were recruited into the National Institutes of Health Epigenetics of Severe Asthma study (n=193) from established cohorts of patients (United Kingdom (UK)): the Wessex AsThma CoHort of difficult asthma (WATCH) at University Hospital Southampton Foundation Trust UK(31) (n=501) consisting of subjects with severe/difficult-to treat asthma (GINA management steps 4 and 5 (33)) and the Isle of Wight Whole Population Birth Cohort (IOWBC) at the David Hide Asthma and Allergy Research Centre, Isle of Wight, UK(121) (n=1,456), along with clinic/community recruitment on the Isle of Wight, UK, of mild asthma subjects (GINA management steps 1 and 2, with a small proportion with step 3). This study received approval from the South Central Hampshire B-Southampton Research Ethics Committee, UK (REC reference: 18/SC/0105) and from the La Jolla Institute for Immunology Institutional Review Board (IRB VD-156-1118, La Jolla Institute for Immunology, La Jolla, USA). Written informed consent was obtained from all subjects. Briefly, subjects underwent an extensive clinical characterization process including detailed clinical, health and disease-related questionnaires, anthropometry, allergy skin prick testing, lung function testing. Subjects with mild (GINA 1 to 3, n=14) and severe asthma (GINA 4 and 5, n=16) subjects underwent fiberoptic bronchoscopy for collection of BAL samples. Mild asthmatic patients were treated with inhaled bronchodilators alone (Salbutamol 200 μg as required) (n=4) and/or with low to medium dose of inhaled corticosteroids (400 to 800 μg/day beclomethasone dipropionate (BDP) equivalent, n=10). Conversely, all severe asthmatic patients were treated with high dose inhaled corticosteroids (1,200 to 2000 μg/day BDP equivalent) and second controller medication (n=14), and/or on daily maintenance oral steroids (n=5) and/or biological monoclonal antibody treatment (Omalizumab (anti-IgE), n=2; Mepolizumab (anti-IL-5), n=7).

### BAL Samples Processing

BAL fluid was obtained by instilling a total volume of 120 mL of warm 0.9% saline in small aliquots (initially 40 mL followed by 20 mL, holding for 10 seconds each time) into the right upper lobe segments using a fiberoptic bronchoscope procedure (n=30). Aliquots were pooled together and collected as 1 sample with immediate storage on ice. The median recovery of BAL volume was 53 mL (Inter quartile range: 24-60 mL). RNAse inhibitor (v:v 1:1000, Takara Bio) and protease inhibitor (v:v 1:50; Sigma Aldrich) were immediately added to BAL collected. BAL was then filtered within 30 minutes with a 100 μm BD cell strainer and centrifugated at 300×g for 10 minutes at 4° C. Cellular fractions were resuspended in 1 mL of phosphate buffer solution (PBS) with RNAse inhibitor (v:v 1:100). Two Cytospin slides were generated with 70 μL of cell suspension using a Shandon Cytospin 2 and stained using rapid Romanowsky (Diff-Quick) stain to obtain differential cell counts and to ascertain the volume of squamous cell contamination(31). Samples were centrifugated once more at 300×g for 10 minutes at 4° C. Supernatants were discarded and cell pellets resuspended into freezing media (50% human decomplemented AB Serum (Sigma Aldrich), 40% complete Gibco Roswell Park Memorial Institute (RPMI) medium (ThermoFisher Scientific) complemented with 10% heat-inactivated fetal bovine serum (FBS) (Sigma Aldrich, 10% DMSO (Sigma Aldrich), and 5 μL of RNAse inhibitor, before slow cryopreserving at −80° C. as described previously(42).

### Flow Cytometry of Cryopreserved BAL Cellular Samples

Cryopreserved samples were thawed, and cells were transferred first to 1 mL of cold heat-inactivated FBS and then quickly diluted up to 10 mL with complete T_{CM} medium (Gibco Iscove's Modified Dulbecco's Medium (IMDM) with 5% FBS and 2% human serum; ThermoFisher Scientific). Samples were centrifugated at 250×g for 5 minutes at room temperature. Supernatants were discarded and cells resuspended in appropriate volume of MACS buffer (PBS, 2 mM EDTA, 2% heat-inactivated FBS) to reach 2 million cells per mL. Around 25% of the sample or maximum of 500,000 cells were separated for stimulation assays. Remaining cells were centrifuged at 400×g for 5 minutes at room temperature, supernatant was discarded, and cells resuspended in 200 μL of MACS buffer complemented with 1% RNAse inhibitor. All samples (resting or after stimulation) were stained following a standard procedure previously described(42). Briefly, 200 μL cell suspensions were first incubated with 20 μL of FcgR blocking solution (Miltenyi Biotec) for 15 minutes on ice, and subsequently stained with the following combination of fluorescently-conjugated antibodies: anti-CD45-Alexa Fluor 700 (2D1; BioLegend), anti-CD3-APC-Cy7 (SK7; BioLegend), anti-CD8a-BV570 (RPA-T8; BioLegend), anti-CD4-BV510 (RPA-T4; BioLegend), anti-CD357 (GITR)-BV711 (108-17; BioLegend), anti-CD185 (CXCR5)-BV421 (RF8B2; BD Biosciences), anti-CD25-BB515 (2A3; BD Biosciences), anti-CD127-APC (eBioRDR5; eBioscience), anti-CD69-BV605 (FN50; BioLegend) and anti-CD103-PE-Cy7 (Ber-ACT8; BioLegend). The Brilliant Stain Buffer Plus (BD Horizon) was also added to the antibody mix as recommended. For a fraction of the samples, the DNA-oligonucleotide anti-CD103 (TotalSeq-C0145; Ber-ACT8; BioLegend) and anti-CD69 antibodies (TotalSeq-C0146; FN50; BioLegend) were also added. After 20 minutes incubation in the dark, on ice, cells were washed once with 5 mL of ice-cold MACS buffer, centrifugated at 400×g for 5 minutes at room temperature (RT), resuspended in 250 μL MACS buffer with RNAse inhibitor (10%), and brought to flow cytometry for immunophenotyping analyses and sorting. Live and dead cells were discriminated using propidium iodide (PI, 1:200 vol:vol). All stained samples were analyzed using BD FACSAria Fusion Cell Sorter (BD Biosciences) and FlowJo software (10.7.1).

### Stimulation Assays

Maximum 25% of BAL samples and no more than 500,000 cells from each BAL sample were stimulated ex vivo in 1 mL of T_{CM} complemented with PMA (final 20 nM, phorbol-12-myristate-13-acetate) and ionomycin (final 1 μM; Sigma Aldrich) for 2 hours in a cell culture incubator at 37° C. and 5% CO₂. Samples were then processed and stained for flow-cytometry analysis and sorting as described here above.

### Cell Isolation for Bulk and Single-Cell RNA-Seq Assay

For bulk RNA-seq assays, cells of interest were directly collected by sorting 400 cells into 0.2 mL PCR tubes (low-retention, Axygen) containing 8 μL of ice-cold lysis buffer (Triton X-100 [0.1%, Sigma-Aldrich], 1% RNase inhibitor [Takara Bio]). Once collected, tubes were vortexed for 10 seconds, spun for 1 minute at 3000×g and directly stored at −80° C. For single-cell RNA-seq assays (10× Genomics), 1,000 to 2,000 airway CD4⁺ T cells were sorted per BAL sample directly in low retention and sterile ice-cold 1.5 mL collection tubes containing 500 μL of PBS: FBS (1:1 vol:vol) with RNAse inhibitor (1:100). Samples were batched in groups of 5 to 6 donors with similar disease status. Samples were also separated based on stimulation. In total, the inventors performed 6 sorting experiments. Collection tubes with ~10,000 to 20,000 sorted CD4⁺ T cells were inverted a few times, ice-cold PBS was added to reach a volume of 1,400 μL, and tubes were centrifuged for 5 minutes at 600×g and 4° C. Supernatant was removed with caution, leaving a volume of around 10 μL. Pellets were then resuspended with 35 μL of 10× Genomics resuspension buffer (0.22 μm filtered ice-cold PBS supplemented with ultra-pure bovine serum albumin (0.04%, Sigma-Aldrich). 40 μL of cell suspension were transferred to an 8 PCR-tube strip for downstream steps as per manufacturer's instructions (10× Genomics).

### Bulk RNA Library Preparation for Sequencing

For full-length bulk transcriptome analyses, the inventors used the Smart-seq2 protocol (adapted for samples with small cell numbers)(122-124). Briefly, RNA was captured using oligo-poly(dT)-3′ primers and reverse transcription was performed using 5′-template switching oligos (LNA technologies, Exicon). cDNA was pre-amplified by PCR cycle for 20 cycles. Amplified cDNA was cleaned by applying a double size purification (0.6 vol:vol and 0.8 vol:vol with Ampure-XP magnetic beads (Beckman Coulter)). After quantification and quality assessment using capillary electrophoresis (Fragment analyzer, Advance analytical), 0.5 ng of pre-amplified cDNA was used to generate indexed Illumina libraries (Nextera XT library preparation kit, Illumina). Every sample was quality checked for fragment size by capillary electrophoresis (Fragment analyzer, Advance analytical) and quantity (Picogreen, Thermofisher). No libraries failed the quality control check steps and therefore were pooled at equal molar concentration before loading on the NovaSeq 6000 Illumina sequencing platform. Every library was sequenced to reach a minimum of 15 million 100×100 bp pair-ended sequencing reads (S4 flowcell 200 cycle v1.0, Xp workflow; Illumina).

### 10× Genomics Single-Cell RNA Library Preparation for Sequencing

Samples were processed using 10× Genomics 3v3.0 single cell gene expression profiling chemistry as per manufacturer's recommendations; after droplet generation, and in-droplet based reverse transcription, cDNA was amplified by PCR for 11 cycles and gene expression library preparation followed. After quantification, equal molar concentration of each library was pooled and sequenced using the NovaSeq6000 Illumina sequencing platform to obtain 28- and 100-bp paired-end reads using the following read length: read 1, 100 cycles; read 2, 100 cycles; i7 index, 8 cycles and i5 index 8 cycles.

For samples stained with DNA-oligo-conjugated-cell-surface antibodies (TotalSeq-C, Biolegend), amplified DNA generated from antibody-DNA oligos was separated from transcriptomic cDNA based on size-selection following amplification. Antibody-DNA amplified fragment are less than 300 bp. Library preparation were followed in accordance with manufacturer's recommendations. TotalSeq libraries were quantified and sequenced in the same manner as the gene expression libraries, as described above. Each library was sequenced aiming 5,000 reads per cell.

### Genotyping

For each patient, genomic DNA was isolated from PBMC using the DNeasy Blood and Tissue Kit (Qiagen) and utilized for genotyping using the Infinium Multi-Ethnic Global-8 Kit (Illumina) following the manufacturer's instructions. Chip-arrays were run on an Illumina iScan System using the University of California—San Diego, Institute of Genomic Medicine. Raw data from the genotyping analysis, data quality assessment and SNPs identification were performed as previously described(42).

### Bulk RNA-Seq Analysis

Bulk RNA-seq data were mapped against the hg19 genome reference using the inventors' in-house pipeline (https://github.com/ndu-UCSD/LJI_RNA_SEQ_PIPELINE_V2). Briefly, FASTQ data from sequencing was merged and filtered using fastp (v0.20.1), reads were aligned with the STAR aligner (v2.7.3a), followed by further processing with samtools (v0.1.19-44428cd), bamCoverage (v3.3.1), and Qualimap (vv.2.2.2-dev). Raw and transcripts per million reads (TPM) counts were taken from STAR's BAM aligned output.

To identify genes expressed differentially between groups, the inventors performed negative binomial tests for paired comparisons by employing DESeq2 (125) (1.16.1) with default parameters and batch and sex as a covariate. The inventors considered genes to be expressed differentially by any comparison when the DESeq2 analysis resulted in a Benjamini-Hochberg-adjusted P-value of less than 0.01 and a log₂ fold change of at least 1.

### Single-Cell RNA-Seq Analysis

Analysis of 3′ transcriptome of single-cell from 10× Genomics platform. Raw data was processed as previously described (29, 44, 46), merging multiple sequencing runs using Cell Ranger's count function, then aggregating multiple cell types with aggr (v3.1.0) (126).

Doublet cell filtering and donor labeling. Barcoded single-cell RNA-seq was demultiplexed patient-wise using Demuxlet (127) with the following parameters: alpha=0, 0.5 and --geno-error=0.05. Each cell was assigned a donor ID or marked as a doublet. Cells called as doublet by Demuxlet were removed from downstream analyses. The inventors did not observe major changes in singlets/doublets proportions between the different 10× Genomics libraries, suggesting optimal processing of cells during 10× (Gel Bead-In emulsions) GEM generation and downstream steps. All downstream analyses were performed using cells labelled as singlets.

CD4⁺ cells selection from stimulated CD3⁺ library. Differential gene expression analysis was performed between filtered CD4⁺ CD8B⁻ and CD4⁻ CD8B⁺ cells from the CD3⁺ library, and differentially expressed genes were used for clustering. Genes were selected with a Benjamini-Hochberg adjusted P-value less than 0.05, log₂ |Fold Change| higher than 2 and sex-related genes were excluded (RPS4Y1, XIST, SPRY1, DDX3Y). Two clusters were identified correlating with the CD4 and CD8 T cell types from which only the CD4⁺ was taken for downstream analysis of stimulated data.

Transcriptome-based clustering analysis. The merged data was transferred to the R statistical environment for analysis. Unbiased clustering analysis was performed using Seurat (v3.0.2)(128). A first round of analysis was run and single-cell transcriptomes not meeting quality control thresholds (see below) as well as a cluster of contaminating cells characterized by a strong monocyte/macrophage signature were eliminated from the second round of analysis. For both analyses the following criteria was applied. Only cells expressing between 200 and 6,000 genes, less than 30,000 total unique molecule identifier (UMI) content, and less than 15% of reads mapping to mitochondria genome, were included. Only genes expressed in at least 0.1% of the cells were included in the analysis. Expression counts were then log-normalized and scaled (by a factor of 10,000) per cell. Variable genes were detected with the VST method and the top highly expressed (UMI mean greater than 0.01) genes representing 15% of the variance were selected for cluster analysis. Transcriptomic data from each cell was then further scaled by regressing the number of UMI-detected and the percentage of mitochondrial reads. Principal component analysis (PCA) was then run on the variable genes, and the first 20 and 17 principal components for resting and stimulated data, respectively, were selected for downstream analyses based on the standard deviation of PCs (“elbow plot”) (FIG. 6C). Cells were clustered using Seurat's functions FindNeighbors and FindClusters with a resolution of 0.4. For the resting condition, the inventors performed downstream analyses excluding a cluster (TAPOPTOSIS) (<2% of airway CD4⁺ T cells) enriched in apoptosis signature genes (as reported by GSEA) and interpreted as a technical artefact (FIG. 6H).

Gene-set score calculation and gene set enrichment analysis (GSEA). Signature scores were calculated with AddModuleScore function from Seurat with default settings. The score is derived from the mean of the gene list after subtracting a background expression calculated from a random list of genes (same size as the gene set). The normalized GSEA enrichment score was calculated using fgsea (v 1.10.1) in R with the signal-to-noise ratio as a metric (129). Default parameters were used except minSize=3 and maxSize=500.

Protein expression analysis using DNA-oligo-antibody single-cell sequencing. TotalSeq-C reads were analyzed based on recommendation provided by manufacturer (BioLegend).

Single-cell differential gene expression analysis. Pairwise analyses were performed using the R package MAST (v 1.8.2)(130) with cellular detection rate (CDR) as a covariate and taking the same number of cells in any given comparison, after normalizing the data to log₂ counts per million (log₂ (CPM+1)). A gene was considered as differentially expressed if its Benjamini-Hochberg adjusted P-value was <0.05 and log₂ (|fold change|) was >0.25 (otherwise noted in legends). Cluster specific markers were determined by MAST using the Seurat function FindAllMarkers with default parameters.

Violin plots represent the distribution of expression (based on a Gaussian Kernel density estimation model) of cells including cells with no expression. To note, with stimulated cells, violin plots only include cells with an expression >0 CPM. Violins are Shaded according to the percentage of cell expressing the transcript of interest.

Crater plots are scatter plots that depict fold change (log₂) of expression for all transcripts from two distinct comparisons, each comparison representing one axis. Every dot is a given transcript, with the size representing the average of both significance values [−log₁₀ (adjusted P-value)] and the shade representing the average level of expression for all cells analyzed.

Volcano plots represent the differentially expressed transcripts with the shade showing the average expression (log₂) derived from the group in which the gene is up-regulated and the size showing the difference in percentage of expressing cells between groups.

### Statistical Analysis.

For RNA-seq data analysis, statistical methods have been described here above. The inventors used unpaired non-parametric T test (Mann-Whitney) for analysis of cell proportions. For correlation analysis with clinical features, as the data used was either ordinal and/or non-linearly distributed, the inventors used Spearman correlation coefficients followed by Bonferroni-Hochberg correction. Correlation trendlines were drawn by simple linear regression. The inventors used GraphPad Prism 9.0.1.

### Patient Cohort Description

Subjects diagnosed with asthma were recruited into the National Institutes of Health Epigenetics of Severe Asthma study (n=193) from established cohorts of patients in (United Kingdom (UK)) the Wessex AsThma CoHort of difficult asthma (WATCH) at University Hospital Southampton Foundation Trust UK22 (n=501) consisting of subjects with severe/difficult-to treat asthma (GINA management steps 4 and 5 24) and the Isle of Wight Whole Population Birth Cohort (IOWBC) at the David Hide Asthma and Allergy Research Centre, Isle of Wight, UK57 (n=1,456), along with clinic/community recruitment on the Isle of Wight, UK, of mild asthma subjects (GINA management steps 1 and 2, with a small proportion with step 3). This study received approval from the South Central Hampshire B—Southampton Research Ethics Committee, UK (REC reference: 18/SC/0105) and from the La Jolla Institute for Immunology Institutional Review Board (IRB VD-156-1118, La Jolla Institute for Immunology, La Jolla, USA). Written informed consent was obtained from all subjects. Briefly, subjects underwent an extensive clinical characterization process including detailed clinical, health and disease-related questionnaires, anthropometry, allergy skin prick testing and, lung function testing. Subjects with mild (GINA 1 to 3, n=14) and severe asthma (GINA 4 and 5, n=16) subjects underwent fibreoptic bronchoscopy for collection of BAL samples. Mild asthmatic patients were treated with inhaled bronchodilators alone (Salbutamol 200 μg as required) (n=4) and/or with low to medium dose of inhaled corticosteroids (400 to 800 μg/day beclomethasone dipropionate (BDP) equivalent, n=10). Conversely, all severe asthmatic patients were treated with high dose inhaled corticosteroids (1,200 to 2000 μg/day BDP equivalent) and second controller medication (n=14), and/or on daily maintenance oral steroids (n=5) and/or biological monoclonal antibody treatment (Omalizumab (anti-IgE), n=2; Mepolizumab (anti-IL-5), n=7).

### Tissue Sampling and Processing

Biopsies tissue sections were kept and stored in 1 mL of complete Gibco Roswell Park Memorial Institute (cRPMI) medium (10% FBS in RPMI 1640 with L-glutamine) after bronchoscopic collection. Within 2 hours, biopsies were enzymatically dispersed with addition of 5 μL of Liberase DL (Roche Diagnostic GmbH) and 5 μL per mL of RNAse inhibitor (v:v 1:1000, Takara Bio) to the tissue solution, for 15 mins at 37° C. on an orbital shaker at 250 rpm. The reaction was stopped by addition of protease inhibitor (v:v 1:50; Sigma Aldrich) and sample passed through a 70 μm cell strainer (BD Biosciences) to obtain a single cell suspension. Sample volume was then made up to 5 mL with cRPMI media. Broncho-alveolar lavage fluid was obtained by instilling a total volume of 120 mL of warm 0.9% saline in small aliquots (initially 40 mL followed by 20 mL, holding for 10 seconds each time) into the right upper lobe segments using a fibreoptic bronchoscope procedure (n=30). Aliquots were pooled together and collected as 1 sample with immediate storage on ice. The median recovery of BAL volume was 53 mL (Inter quartile range: 24-60 mL). RNAse inhibitor (v:v 1:1000, Takara Bio) and protease inhibitor (v:v 1:50; Sigma Aldrich) were immediately added to BAL collected. BAL was then filtered within 30 minutes with a 100 μm BD cell strainer. All samples were centrifuged for 10 minutes at 400×g at 4° C. BAL supernatants were collected, aliquoted, and stored at −80° C. Cellular fractions for BALs and Biopsies were then resuspended in 1 mL of phosphate buffer solution (PBS) with RNAse inhibitor (v:v 1:100). Two Cytospin slides were generated with 70 μL of cell suspension using a Shandon Cytospin 2 and stained using rapid Romanowsky (Diff-Quick) stain to obtain differential cell counts and to ascertain the volume of squamous cell contamination22. Samples were centrifugated once more at 300×g for 10 minutes at 4° C. Supernatants were discarded and cell pellets resuspended into freezing media (50% human decomplemented AB Serum (Sigma Aldrich), 40% complete Gibco Roswell Park Memorial Institute (RPMI) medium (ThermoFisher Scientific) complemented with 10% heat-inactivated fetal bovine serum (FBS) (Sigma Aldrich, 10% DMSO (Sigma Aldrich), and 5 μL of RNAse inhibitor, before slow cryopreserving at −80° C. as described previously (188).

### Flow Cytometry

Samples were rapidly thawed (less than a minute), and transferred to a 15 mL conical tube containing 1 mL of cold heat inactivated FBS. Samples volumes were made up to 10 mL with complete TCM medium (Gibco Iscove's Modified Dulbecco's Medium (IMDM), 5% FBS, 2% human serum; ThermoFisher Scientific) and tubes centrifugated at 250×g for 5 minutes at room temperature. Cell pellets were homogenized with MACS buffer (PBS, 2 mM EDTA, 2% heat-inactivated FBS) to reach 2 million cells per mL. For BAL cells only, samples were split into 2 fractions based on a 25 (but maximum of 500,000 cells): 75 ratio. The 25% fraction was used for stimulation assays. Cell from the remaining fraction were centrifuged at RT, for 5 minutes at 400×g and directly resuspended in 200 μL of MACS buffer+RNAse inhibitor (1:100 v:v). Staining procedure was followed as previously described, first, samples were incubated with 20 μL of FcgR blocking antibody (Miltenyi Biotec) for 15 minutes on ice, and subsequently stained with a cocktail of FACS-labelled antibodies: anti-CD45-Alexa Fluor 700 (2D1; BioLegend), anti-CD3-APC-Cy7 (SK7; BioLegend), anti-CD8a-BV570 (RPA-T8; BioLegend), anti-CD4-BV510 (RPA-T4; BioLegend), anti-CD357 (GITR)-BV711 (108-17; BioLegend), anti-CD185 (CXCR5)-BV421 (RF8B2; BD Biosciences), anti-CD25-BB515 (2A3; BD Biosciences), anti-CD127-APC (eBioRDR5; eBioscience), anti-CD69-BV605 (FN50; BioLegend) and anti-CD103-PE-Cy7 (Ber-ACT8; BioLegend). The inventors also added the Brilliant Stain Buffer Plus (BD Horizon) as recommended. For certain samples, the inventors added DNA-oligonucleotide anti-CD103 (TotalSeq-C0145; Ber-ACT8; BioLegend) and anti-CD69 antibodies (TotalSeq-C0146; FN50; BioLegend). Samples were incubated in obscurity and ice for 20 minutes then washed once with 5 mL of ice-cold MACS buffer before to be centrifugated (400×g/5 min/RT) and resuspended in 250 μL of MACS+RNAse inhibitor (10%). Right before analysis, propidium iodide (PI, 1:200 vol:vol) was added to discriminate live and dead cells. Analysis and sorting were performed using BD FACSAria Fusion Cell Sorter (BD Biosciences) and FlowJo software (10.7.1).

### Stimulation Assay

A maximum 25% of BAL samples and no more than 500,000 cells from each BAL sample were stimulated ex vivo in 1 mL of TCM complemented with PMA (final 20 nM, phorbol-12-myristate-13-acetate) and ionomycin (final 1 μM; Sigma Aldrich) for 2 hours at 37° C. in a 5% CO₂ incubator. Samples were processed as described here above.

### Cell Isolation for Single-Cell RNA-Seq Assay

For single-cell RNA-seq assays (10× Genomics), 1,000 to 2,000 CD8+ T cells were sorted per sample directly in a low retention and sterile ice-cold 1.5 mL collection tube containing 500 μL of PBS: FBS (1:1 vol:vol)+RNAse inhibitor (1:100 vol:vol). 5 to 6 donors from identical disease group/stimulation condition were sorted at once and all cells collected pooled together. Around 10,000 to 20,000 sorted CD8+ T cells were collected in a low-retention 1.5 mL tube, volume brought to 1400 μL with ice-cold PBS+RNAse inhibitor and centrifuged (5 minutes, 600×g, 4° C.). Supernatant was removed with caution, leaving a volume of around 10 μL. Cells were gently resuspended with 35 μL of 10× Genomics resuspension buffer (0.22 μm filtered ice-cold PBS supplemented with ultra-pure bovine serum albumin (0.04%, Sigma-Aldrich). Out of the 45 μL of cell suspension, 40 μL were loaded on 10× Genomic ChIP and processed per manufacturer's instructions (10× Genomics).

### Library Preparation for Single-Cell Sequencing

For gene expression, the inventors used 3v3.0 single cell gene expression profiling chemistry kits (10× Genomics). First, reverse-transcription happened directly after cells oil-droplet encapsulation. cDNA went through 11 cycles of linear PCR amplification and library preparation followed. Final libraries were quantified as recommended and molar concentration of each library determined. Libraries were pooled and sequenced using the NovaSeq6000 Illumina sequencing platform to obtain 28- and 100-bp paired-end reads per cell using the following read length: read 1, 100 cycles; read 2, 100 cycles; i7 index, 8 cycles and i5 index 8 cycles. For samples stained with DNA-oligo-conjugated-cell-surface antibodies (TotalSeq-C, Biolegend), amplified DNA fragments (less than 300 bp) generated from antibody-DNA oligos were separated from transcriptomic cDNA by size-selection in accordance with manufacturer's recommendations. Libraries were quantified and sequenced to obtain 5,000 reads per cell. For Biopsy T cells samples (in the CD45+ sorted cellular compartment), the inventors also prepared libraries to collect TCR-α and TCR-β chains sequence information.

### 10× Genomics Single-Cell Sequence Analysis

Single-cell gene expression analysis—Sequenced data was processed as previously described^{25,32,63}. Merging of data from multiple sequencing runs were performed using Cell Ranger's count function, and then aggregated with aggr (v3.1.0)⁶⁴.

Doublet cell filtering and donor labelling—The inventors used the Demuxlet algorithm to identify donor specific ID for each single-cell transcriptome, based on genotyping data, as well as to filter out cell doublets [refs Seumois et al. 2020]. Single-cell RNA-seq were demultiplexed using the 10× Genomics cell ranger algorithm, then, following the parameters: alpha=0, 0.5 and --geno-error=0.05, each single-cell transcriptome was assigned a donor ID or marked as a doublet/multiplet, only singlet cell with a valid cell-barcode ID and donor ID were used. There was no high variability in doublet/multiplet proportions across libraries suggesting optimal processing of cells during 10x (Gel Bead-In emulsions) GEM generation and subsequent library preparation steps.

CD8⁺ T cells filtering—For a few libraries, the inventors pooled both populations, CD4⁺ and CD8⁺ T cells together, or sorted all CD3⁺ T cells. To bioinformatically discriminate CD4⁺ from CD8⁺ T cells, taken into consideration the CD4 and CD8A/B transcript drop-outs, the inventors extracted a list of differentially expressed genes between datasets generated from libraries made with exclusive sorted CD4⁺ CD8B⁻ or CD4⁻CD8B⁺ T cells. Differentially Expressed Genes were selected with a Benjamini-Hochberg adjusted P-value less than 0.05, log₂ |Fold Change| higher than 2, and sex-related genes were excluded (RPS4Y1, XIST, SPRY1, DDX3Y). Cells from mixed CD4/CD8 libraries were separated based on a score and only cells enriched for the CD8+DEG and depleted for CD4⁺ DEGs were taken for downstream analysis.

Single-cell transcriptome-based clustering analysis—The matrix was transferred to the R environment for downstream analyses. Using Seurat (v3.0.2), unbiased clustering analysis was performed⁶⁵. First, the inventors performed a first round of transcriptomic quality control assessment. The inventors kept cell barcodes expressing between 200 and 6,000 features (genes), less than 30,000 total unique molecule identifier (UMI) content, and less than 15% of reads mapping to mitochondria features. The inventors also eliminated contaminating cells characterized by strong monocyte/macrophage gene signature. Only features (genes) expressed in at least 0.1% of the total number of cells were included in the downstream analyses. Expression counts for each gene was transformed into log-normalized and scaled (by a factor of 10,000) per single-cell [ref]. Second, to proceed with clustering analysis, the inventors selected the features (gene) having a UMI mean greater than 0.01. Then the inventors selected the top highly variable features (genes), as detected with the VST method, representing 15% of the cumulative variance. Transcriptomic data from each cell barcode was then further scaled by regressing the number of UMI-detected and the percentage of mitochondrial reads. Principal component analysis (PCA) was then run using the highly variable features, and the first 25 and 20 principal components for resting and stimulated data, respectively, were selected for downstream analyses based on the standard deviation of PCs (“elbow plot”). Resting data was clustered using Seurat's functions FindNeighbors and FindClusters with a resolution of 0.6.

Single-cell differential gene expression analysis—Pairwise comparisons were performed using the R package MAST (v 1.8.2) leveraged by the cellular detection rate (CDR) as a covariate and taking the same number of cells in both groups being compared, after normalizing the data to log₂ (counts per million+1) (log₂ (CPM+1)). A gene is considered as differentially expressed if both statistical conditions, Benjamini-Hochberg adjusted P-value was <0.05 and log₂ |fold change| was >0.25 (or otherwise noted in figure legends) were met. Cluster enriched transcripts were determined by MAST using the Seurat function FindAllMarkers with default parameters.

Violin plots—Violin plots represent the distribution (built from a Gaussian Kernel density estimation model) of single cell gene expression for all cells belonging to a given cluster of cells. The scale used depict the percentage of cells expressing the transcript of interest.

Crater plots—Crater plots represent the joint distribution of fold change (log₂) for all transcripts from two distinct differential gene expression analysis comparisons, each comparison representing one axis. Each dot is a single transcript, the size of dot represents the significance value [−log₁₀ (adjusted P-value)] between the two comparisons and the shading depicts the average level of expression for all cells analyzed.

Volcano plots—A volcano plots is a type of scatter plot to illustrate differential gene expression analysis between 2 groups of cells. It shows for every gene analyzed (dot), the statistical significance value [−log₁₀ (adjusted P-value)] (Y-axis) versus the magnitude of change in expression (−log 2(fold change|) (X-axis). The dot shade scale follows the average expression (log₂) of cells from the group in which the gene is up-regulated, and the dot size reflects on the difference in percentage of expressing cells between groups. Pairwise comparisons were performed using the R package MAST (v 1.8.2) leveraged by the cellular detection rate (CDR) as a covariate and taking the same number of cells in both groups being compared, after normalizing the data to log₂ counts per million (log₂(CPM+1))(196).

Gene-set score calculation and gene set enrichment analysis (GSEA) for single-cell transcriptomic data. Module signature scores were calculated with AddModuleScore function from Seurat with default parameters. The score is obtained from the mean of the signature gene list after subtracting the “background” expression calculated from a random list of genes (both the same size). The Normalized Enrichment Score from GSEA was calculated using fgsea (v 1.10.1) in R with the signal-to-noise ratio as a metric (197). Parameters used were minSize=3 and maxSize=500.

Protein expression analysis using DNA-oligo-antibody single-cell sequencing. Single-cell TotalSeq-A reads were analyzed based on recommendation provided by manufacturer (BioLegend). The inventors in-house pipeline was used. Briefly, TotalSeq A reads were pre-processed recovering mismatched antibody barcodes with an error correcting algorithm that scores these reads and assigns them to the closest barcode. Quality controls were performed using a Gaussian distribution to detect outliers as those not within the distribution at a probability of 10⁻⁶.

Single-cell targeted TCR-α and TCR-β chain sequencing analysis—For

Biopsy T cells samples, the inventors processed reads from the V(D)J TCR sequence enriched libraries using the Cell Ranger vdj pipeline (v3.1.0 and human annotations reference GRCh38, as per recommendations. For each independent library the V(D)J transcripts were assembled and their respective annotations. The inventors used a custom script to then aggregate the V(D)J libraries and carryout a combined single cell transcriptome, and TCR sequence analysis. The specific cell barcodes were revised relative to their gene expression libraries and unique clonotypes (productive complementarity-determining region 3 (CDR3) sequences) were identified. The respective library files and clone statistics, i.e. frequency and size proportion, were calculated based on a specific aggregation output and only including cells present in the gene expression libraries. This procedure was used to identify TCR-CDR3 sequences from CD8⁺ T cells from the bronchial endoscopic biopsies. Based on the V(D)J aggregation files, the captured barcodes in the gene expression data and good quality cell filtering, a specific clonotype ID was assigned. The clone size (a number of cells with the same clonotypes in either one or both TCR chains (alpha and beta)) was described for each identified clonotype ID per cluster and per donor. Cells that shared a clonotype with more than 1 cell were classified as clonally expanded (clone size>1). Sharing of clonotypes between cells in the different clusters was depicted using the tool UpSetR⁶⁸. To assess clonotype sharing between BAL derived CD8⁺ and Bronchial biopsy CD8+ T cells, the same aggregation process was implemented for all of the vdj libraries specifically isolated from these data and the inventors considered CD8⁺ T cells isolated from matched patients between sets.

### Statistical Analysis

For RNA-seq data analysis, statistical methods have been described here above. The inventors used unpaired non-parametric T test (Mann-Whitney) for analysis of cell proportions. For correlation analysis with clinical features, as the data used was either ordinal and/or non-linearly distributed, the inventors used Spearman correlation coefficients followed by Bonferroni-Hochberg correction. Correlation trendlines were drawn by simple linear regression. GraphPad Prism 9.0.1 was used.

### REFERENCES

1. S. C. Dharmage, J. L. Perret, A. Custovic, Epidemiology of Asthma in Children and Adults. Front Pediatr 7, 246 (2019).
2. H. Hammad, B. N. Lambrecht, The basic immunology of asthma. Cell 184, 2521-2522 (2021).
3. B. León, A. Ballesteros-Tato, Modulating Th2 Cell Immunity for the Treatment of Asthma. Frontiers in Immunology 12, (2021).
4. Centers for Disease Control and Prevention. 2019 National Health Interview Survey Data. U.S. Department of Health & Human Services. (2020). https://www.cdc.gov/asthma/nhis/2019/data.htm
5. K. P. Hough, M. L. Curtiss, T. J. Blain, R.-M. Liu, J. Trevor, J. S. Deshane, V. J. Thannickal, Airway Remodeling in Asthma. Frontiers in Medicine 7, (2020).
6. B. N. Lambrecht, H. Hammad, J. V. Fahy, The Cytokines of Asthma. Immunity 50, 975-991 (2019).
7. G. Seumois, J. Zapardiel-Gonzalo, B. White, D. Singh, V. Schulten, M. Dillon, D. Hinz, D. H. Broide, A. Sette, B. Peters, P. Vijayanand, Transcriptional Profiling of Th2 Cells Identifies Pathogenic Features Associated with Asthma. The Journal of Immunology 197, 655-664 (2016).
8. R. Wadhwa, K. Dua, I. M. Adcock, J. C. Horvat, R. Y. Kim, P. M. Hansbro, Cellular mechanisms underlying steroid-resistant asthma. European Respiratory Review 28, 190096 (2019).
9. J. M. Drazen, Asthma: the paradox of heterogeneity. J Allergy Clin Immunol 129, 1200-1201 (2012).
10. K. F. Chung, S. E. Wenzel, J. L. Brozek, A. Bush, M. Castro, P. J. Sterk, I. M. Adcock, E. D. Bateman, E. H. Bel, E. R. Bleecker, L.-P. Boulet, C. Brightling, P. Chanez, S.-E. Dahlen, R. Djukanovic, U. Frey, M. Gaga, P. Gibson, Q. Hamid, N. N. Jajour, T. Mauad, R. L. Sorkness, W. G. Teague, International ERS/ATS guidelines on definition, evaluation and treatment of severe asthma. European Respiratory Journal 43, 343-373 (2014).
11. M. Castro, J. Corren, I. D. Pavord, J. Maspero, S. Wenzel, K. F. Rabe, W. W. Busse, L. Ford, L. Sher, J. M. FitzGerald, C. Katelaris, Y. Tohda, B. Zhang, H. Staudinger, G. Pirozzi, N. Amin, M. Ruddy, B. Akinlade, A. Khan, J. Chao, R. Martincova, N. M. H. Graham, J. D. Hamilton, B. N. Swanson, N. Stahl, G. D. Yancopoulos, A. Teper, Dupilumab Efficacy and Safety in Moderate-to-Severe Uncontrolled Asthma. New England Journal of Medicine 378, 2486-2496 (2018).
12. H. G. Ortega, M. C. Liu, I. D. Pavord, G. G. Brusselle, J. M. FitzGerald, A. Chetta, M. Humbert, L. E. Katz, O. N. Keene, S. W. Yancey, P. Chanez, Mepolizumab Treatment in Patients with Severe Eosinophilic Asthma. New England Journal of Medicine 371, 1198-1207 (2014).
13. M. Humbert, C. Taillé, L. Mala, V. Le Gros, J. Just, M. Molimard, Omalizumab effectiveness in patients with severe allergic asthma according to blood eosinophil count: the STELLAIR study. European Respiratory Journal 51, 1702523 (2018).
14. W. Liu, S. Liu, M. Verma, I. Zafar, J. T. Good, D. Rollins, S. Groshong, M. M. Gorska, R. J. Martin, R. Alam, Mechanism of TH2/TH17-predominant and neutrophilic TH2/TH17-low subtypes of asthma. Journal of Allergy and Clinical Immunology 139, 1548-1558.e1544 (2017).
15. S. Koch, N. Sopel, S. Finotto, Th9 and other IL-9-producing cells in allergic asthma. Seminars in Immunopathology 39, 55-68 (2017).
16. T. Zhou, X. Huang, Y. Zhou, J. Ma, M. Zhou, Y. Liu, L. Xiao, J. Yuan, J. Xie, W. Chen, Associations between Th17-related inflammatory cytokines and asthma in adults: A Case-Control Study. Scientific Reports 7, 15502 (2017).
17. J. Cui, S. Pazdziorko, J. S. Miyashiro, P. Thakker, J. W. Pelker, C. Declercq, A. Jiao, J. Gunn, L. Mason, J. P. Leonard, C. M. Williams, S. Marusic, TH1-mediated airway hyperresponsiveness independent of neutrophilic inflammation. J Allergy Clin Immunol 115, 309-315 (2005).
18. A. Sahoo, A. Alekseev, K. Tanaka, L. Obertas, B. Lerman, C. Haymaker, K. Clise-Dwyer, J. S. McMurray, R. Nurieva, Batf is important for IL-4 expression in T follicular helper cells. Nature Communications 6, 7997 (2015).
19. B. Bošnjak, S. Kazemi, L. M. Altenburger, G. Mokrović, M. M. Epstein, Th2-T (RMs) Maintain Life-Long Allergic Memory in Experimental Asthma in Mice. Front Immunol 10, 840 (2019).
20. C. M. Lloyd, E. M. Hessel, Functions of T cells in asthma: more than just T (H) 2 cells. Nat Rev Immunol 10, 838-848 (2010).
21. G. Seumois, C. Ramírez-Suástegui, B. J. Schmiedel, S. Liang, B. Peters, A. Sette, P. Vijayanand, Single-cell transcriptomic analysis of allergen-specific T cells in allergy and asthma. Sci Immunol 5, (2020).
22. R. A. Rahimi, K. Nepal, M. Cetinbas, R. I. Sadreyev, A. D. Luster, Distinct functions of tissue-resident and circulating memory Th2 cells in allergic airway disease. J Exp Med 217, (2020).
23. T. Ichikawa, K. Hirahara, K. Kokubo, M. Kiuchi, A. Aoki, Y. Morimoto, J. Kumagai, A. Onodera, N. Mato, D. J. Tumes, Y. Goto, K. Hagiwara, Y. Inagaki, T. Sparwasser, K. Tobe, T. Nakayama, CD103(hi) Treg cells constrain lung fibrosis induced by CD103(lo) tissue-resident pathogenic CD4 T cells. Nat Immunol 20, 1469-1480 (2019).
24. D. L. Turner, M. Goldklang, F. Cvetkovski, D. Paik, J. Trischler, J. Barahona, M. Cao, R. Dave, N. Tanna, J. M. D'Armiento, D. L. Farber, Biased Generation and In Situ Activation of Lung Tissue-Resident Memory CD4 T Cells in the Pathogenesis of Allergic Asthma. J Immunol 200, 1561-1569 (2018).
25. P. A. Szabo, M. Miron, D. L. Farber, Location, location, location: Tissue resident memory T cells in mice and humans. Sci Immunol 4, (2019).
26. T. S. Hinks, X. Zhou, K. J. Staples, B. D. Dimitrov, A. Manta, T. Petrossian, P. Y. Lum, C. G. Smith, J. A. Ward, P. H. Howarth, A. F. Walls, S. D. Gadola, R. Djukanović, Innate and adaptive T cells in asthmatic patients: Relationship to severity and disease mechanisms. J Allergy Clin Immunol 136, 323-333 (2015).
27. L. M. Muehling, M. G. Lawrence, J. A. Woodfolk, Pathogenic CD4(⁺) T cells in patients with asthma. J Allergy Clin Immunol 140, 1523-1540 (2017).
28. G. Seumois, P. Vijayanand, Single-cell analysis to understand the diversity of immune cell types that drive disease pathogenesis. J Allergy Clin Immunol 144, 1150-1153 (2019).
29. J. Clarke, B. Panwar, A. Madrigal, D. Singh, R. Gujar, O. Wood, S. J. Chee, S. Eschweiler, E. V. King, A. S. Awad, C. J. Hanley, K. J. McCann, S. Bhattacharyya, E. Woo, A. Alzetani, G. Seumois, G. J. Thomas, A. P. Ganesan, P. S. Friedmann, T. Sanchez-Elsner, F. Ay, C. H. Ottensmeier, P. Vijayanand, Single-cell transcriptomic analysis of tissue-resident memory T cells in human lung cancer. J Exp Med 216, 2128-2149 (2019).
30. F. A. Vieira Braga, G. Kar, M. Berg, O. A. Carpaij, K. Polanski, L. M. Simon, S. Brouwer, T. Gomes, L. Hesse, J. Jiang, E. S. Fasouli, M. Efremova, R. Vento-Tormo, C. Talavera-López, M. R. Jonker, K. Affleck, S. Palit, P. M. Strzelecka, H. V. Firth, K. T. Mahbubani, A. Cvejic, K. B. Meyer, K. Saeb-Parsy, M. Luinge, C. A. Brandsma, W. Timens, I. Angelidis, M. Strunz, G. H. Koppelman, A. J. van Oosterhout, H. B. Schiller, F. J. Theis, M. van den Berge, M. C. Nawijn, S. A. Teichmann, A cellular census of human lungs identifies novel cell states in health and in asthma. Nat Med 25, 1153-1163 (2019).
31. A. Azim, H. Mistry, A. Freeman, C. Barber, C. Newell, K. Gove, Y. Thirlwall, M. Harvey, K. Bentley, D. Knight, K. Long, F. Mitchell, Y. Cheng, J. Varkonyi-Sepp, W. Grabau, P. Dennison, H. M. Haitchi, S. H. Arshad, R. Djukanovic, T. Wilkinson, P. Howarth, R. J. Kurukulaaratchy, Protocol for the Wessex AsThma CoHort of difficult asthma (WATCH): a pragmatic real-life longitudinal study of difficult asthma in the clinic. BMC Pulm Med 19, 99 (2019).
32. K. Larsson, H. Kankaanranta, C. Janson, L. Lehtimäki, B. Ställberg, A. Løkke, K. Høines, K. Roslind, C. S. Ulrik, Bringing asthma care into the twenty-first century. npj Primary Care Respiratory Medicine 30, 25 (2020).
33. Global Initiative for Asthma. Global Strategy for Asthma Management and Prevention. (2021). www.ginasthma.org
34. A. E. Oja, B. Piet, C. Helbig, R. Stark, D. van der Zwan, H. Blaauwgeers, E. B. M. Remmerswaal, D. Amsen, R. E. Jonkers, P. D. Moerland, M. A. Nolte, R. A. W. van Lier, P. Hombrink, Trigger-happy resident memory CD4⁺ T cells inhabit the human lungs. Mucosal Immunology 11, 654-667 (2018).
35. B. V. Kumar, W. Ma, M. Miron, T. Granot, R. S. Guyer, D. J. Carpenter, T. Senda, X. Sun, S. H. Ho, H. Lerner, A. L. Friedman, Y. Shen, D. L. Farber, Human Tissue-Resident Memory T Cells Are Defined by Core Transcriptional and Functional Signatures in Lymphoid and Mucosal Sites. Cell Rep 20, 2921-2934 (2017).
36. K. A. Casey, K. A. Fraser, J. M. Schenkel, A. Moran, M. C. Abt, L. K. Beura, P. J. 36. Lucas, D. Artis, E. J. Wherry, K. Hogquist, V. Vezys, D. Masopust, Antigen-independent differentiation and maintenance of effector-like resident memory T cells in tissues. J Immunol 188, 4866-4875 (2012).
37. K. L. Cepek, S. K. Shaw, C. M. Parker, G. J. Russell, J. S. Morrow, D. L. Rimm, M. B. Brenner, Adhesion between epithelial cells and T lymphocytes mediated by E-cadherin and the alpha E beta 7 integrin. Nature 372, 190-193 (1994).
38. T. I. Arnon, Y. Xu, C. Lo, T. Pham, J. An, S. Coughlin, G. W. Dorn, J. G. Cyster, GRK2-dependent S1PR1 desensitization is required for lymphocytes to overcome their attraction to blood. Science 333, 1898-1903 (2011).
39. J. C. Porter, A. Hall, Epithelial ICAM-1 and ICAM-2 regulate the egression of human T cells across the bronchial epithelium. Faseb j 23, 492-502 (2009).
40. L. Petiti, L. Pace, The persistence of stemness. Nature Immunology 21, 492-494 (2020).
41. A. Y. Rudensky, Regulatory T cells and Foxp3. Immunol Rev 241, 260-268 (2011).
42. B. J. Schmiedel, D. Singh, A. Madrigal, A. G. Valdovino-Gonzalez, B. M. White, J. Zapardiel-Gonzalo, B. Ha, G. Altay, J. A. Greenbaum, G. McVicker, G. Seumois, A. Rao, M. Kronenberg, B. Peters, P. Vijayanand, Impact of Genetic Polymorphisms on Human Immune Cell Gene Expression. Cell 175, 1701-1715.e1716 (2018).
43. M. Locci, C. Havenar-Daughton, E. Landais, J. Wu, M. A. Kroenke, C. L. Arlehamn, L. F. Su, R. Cubas, M. M. Davis, A. Sette, E. K. Haddad, P. Poignard, S. Crotty, Human circulating PD-1⁺CXCR3-CXCR5⁺ memory Tfh cells are highly functional and correlate with broadly neutralizing HIV antibody responses. Immunity 39, 758-769 (2013).
44. G. Seumois, C. Ramírez-Suástegui, B. J. Schmiedel, S. Liang, B. Peters, A. Sette, P. Vijayanand, Single-cell transcriptomic analysis of allergen-specific T cells in allergy and asthma. Science Immunology 5, eaba6087 (2020).
45. J. A. Best, D. A. Blair, J. Knell, E. Yang, V. Mayya, A. Doedens, M. L. Dustin, A. W. Goldrath, Transcriptional insights into the CD8(⁺) T cell response to infection and memory T cell formation. Nat Immunol 14, 404-412 (2013).
46. V. S. Patil, A. Madrigal, B. J. Schmiedel, J. Clarke, P. O'Rourke, A. D. de Silva, E. Harris, B. Peters, G. Seumois, D. Weiskopf, A. Sette, P. Vijayanand, Precursors of human CD4⁺ cytotoxic T lymphocytes identified by single-cell transcriptome analysis. Science Immunology 3, eaan8664 (2018).
47. A. Azim, A. Freeman, A. Lavenu, H. Mistry, H. M. Haitchi, C. Newell, Y. Cheng, Y. Thirlwall, M. Harvey, C. Barber, K. Pontoppidan, P. Dennison, S. H. Arshad, R. Djukanovic, P. Howarth, R. J. Kurukulaaratchy, New Perspectives on Difficult Asthma; Sex and Age of Asthma-Onset Based Phenotypes. The Journal of Allergy and Clinical Immunology: In Practice 8, 3396-3406.e3394 (2020).
48. M. Amelink, S. B. de Nijs, M. Berger, E. J. Weersink, A. ten Brinke, P. J. Sterk, E. H. Bel, Non-atopic males with adult onset asthma are at risk of persistent airflow limitation. Clin Exp Allergy 42, 769-774 (2012).
49. W. G. Teague, B. R. Phillips, J. V. Fahy, S. E. Wenzel, A. M. Fitzpatrick, W. C. Moore, A. T. Hastie, E. R. Bleecker, D. A. Meyers, S. P. Peters, M. Castro, A. M. Coverstone, L. B. Bacharier, N. P. Ly, M. C. Peters, L. C. Denlinger, S. Ramratnam, R. L. Sorkness, B. M. Gaston, S. C. Erzurum, S. A. A. Comhair, R. E. Myers, J. Zein, M. D. DeBoer, A. M. Irani, E. Israel, B. Levy, J. C. Cardet, W. Phipatanakul, J. M. Gaffin, F. Holguin, M. L. Fajt, S. J. Aujla, D. T. Mauger, N. N. Jarjour, Baseline Features of the Severe Asthma Research Program (SARP III) Cohort: Differences with Age. J Allergy Clin Immunol Pract 6, 545-554.e544 (2018).
50. L. K. Mackay, M. Minnich, N. A. Kragten, Y. Liao, B. Nota, C. Seillet, A. Zaid, K. Man, S. Preston, D. Freestone, A. Braun, E. Wynne-Jones, F. M. Behr, R. Stark, D. G. Pellicci, D. I. Godfrey, G. T. Belz, M. Pellegrini, T. Gebhardt, M. Busslinger, W. Shi, F. R. Carbone, R. A. van Lier, A. Kallies, K. P. van Gisbergen, Hobit and Blimp1 instruct a universal transcriptional program of tissue residency in lymphocytes. Science 352, 459-463 (2016).
51. A. E. Oja, F. A. Vieira Braga, E. B. Remmerswaal, N. A. Kragten, K. M. Hertoghs, J. Zuo, P. A. Moss, R. A. van Lier, K. P. van Gisbergen, P. Hombrink, The Transcription Factor Hobit Identifies Human Cytotoxic CD4(⁺) T Cells. Front Immunol 8, 325 (2017).
52. Y. Serroukh, C. Gu-Trantien, B. Hooshiar Kashani, M. Defrance, T. P. Vu Manh, A. Azouz, A. Detavernier, A. Hoyois, J. Das, M. Bizet, E. Pollet, T. Tabbuso, E. Calonne, K. van Gisbergen, M. Dalod, F. Fuks, S. Goriely, A. Marchant, The transcription factors Runx3 and ThPOK cross-regulate acquisition of cytotoxic function by human Th1 lymphocytes. Elife 7, (2018).
53. K. D. Omilusik, J. A. Best, B. Yu, S. Goossens, A. Weidemann, J. V. Nguyen, E. Seuntjens, A. Stryjewska, C. Zweier, R. Roychoudhuri, L. Gattinoni, L. M. Bird, Y. Higashi, H. Kondoh, D. Huylebroeck, J. Haigh, A. W. Goldrath, Transcriptional repressor ZEB2 promotes terminal differentiation of CD8⁺ effector and memory T cell populations during infection. J Exp Med 212, 2027-2039 (2015).
54. I. Albrecht, U. Niesner, M. Janke, A. Menning, C. Loddenkemper, A. A. Kühl, I. Lepenies, M. H. Lexberg, K. Westendorf, K. Hradilkova, J. Grün, A. Hamann, J. A. Epstein, H. D. Chang, K. Tokoyoda, A. Radbruch, Persistence of effector memory Th1 cells is regulated by Hopx. Eur J Immunol 40, 2993-3006 (2010).
55. R. Tippalagama, A. Singhania, P. Dubelko, C. S. Lindestam Arlehamn, A. Crinklaw, M. Pomaznoy, G. Seumois, A. D. deSilva, S. Premawansa, D. Vidanagama, B. Gunasena, N. D. S. Goonawardhana, D. Ariyaratne, T. J. Scriba, R. H. Gilman, M. Saito, R. Taplitz, P. Vijayanand, A. Sette, B. Peters, J. G. Burel, HLA-DR Marks Recently Divided Antigen-Specific Effector CD4 T Cells in Active Tuberculosis Patients. J Immunol, (2021).
56. R. M. Berbers, M. M. van der Wal, J. M. van Montfrans, P. M. Ellerbroek, V. Dalm, P. M. van Hagen, H. L. Leavis, F. van Wijk, Chronically Activated T-cells Retain Their Inflammatory Properties in Common Variable Immunodeficiency. J Clin Immunol, (2021).
57. S. Makris, M. Paulsen, C. Johansson, Type I Interferons as Regulators of Lung Inflammation. Front Immunol 8, 259 (2017).
58. I. W. Choi, K. Sun, Y. S. Kim, H. M. Ko, S. Y. Im, J. H. Kim, H. J. You, Y. C. Lee, J. H. Lee, Y. M. Park, H. K. Lee, TNF-alpha induces the late-phase airway hyperresponsiveness and airway inflammation through cytosolic phospholipase A(2) activation. J Allergy Clin Immunol 116, 537-543 (2005).
59. T. A. Doherty, P. Soroosh, N. Khorram, S. Fukuyama, P. Rosenthal, J. Y. Cho, P. S. Norris, H. Choi, S. Scheu, K. Pfeffer, B. L. Zuraw, C. F. Ware, D. H. Broide, M. Croft, The tumor necrosis factor family member LIGHT is a target for asthmatic airway remodeling. Nat Med 17, 596-603 (2011).
60. R. Lang, F. A. M. Raffi, Dual-Specificity Phosphatases in Immunity and Infection: An Update. Int J Mol Sci 20, (2019).
61. R. Lang, M. Hammer, J. Mages, DUSP meet immunology: dual specificity MAPK phosphatases in control of the inflammatory response. J Immunol 177, 7497-7504 (2006).
62. E. Verjans, K. Ohl, L. K. Reiss, F. van Wijk, A. A. Toncheva, A. Wiener, Y. Yu, A. D. Rieg, V. D. Gaertner, J. Roth, E. Knol, M. Kabesch, N. Wagner, S. Uhlig, C. Martin, K. Tenbrock, The CAMP response element modulator (CREM) regulates TH2 mediated inflammation. Oncotarget 6, 38538-38551 (2015).
63. T. Koga, C. M. Hedrich, M. Mizui, N. Yoshida, K. Otomo, L. A. Lieberman, T. Rauen, J. C. Crispín, G. C. Tsokos, CaMK4-dependent activation of AKT/mTOR and CREM-α underlies autoimmunity-associated Th17 imbalance. J Clin Invest 124, 2234-2245 (2014).
64 M. Giordano, R. Roncagalli, P. Bourdely, L. Chasson, M. Buferne, S. Yamasaki, R. Beyaert, G. van Loo, N. Auphan-Anezin, A.-M. Schmitt-Verhulst, G. Verdeil, The tumor necrosis factor alpha-induced protein 3 (TNFAIP3, A20) imposes a brake on antitumor activity of CD8 T cells. Proceedings of the National Academy of Sciences 111, 11115-11120 (2014).
65. N. Weathington, M. E. O'Brien, J. Radder, T. C. Whisenant, E. R. Bleecker, W. W. Busse, S. C. Erzurum, B. Gaston, A. T. Hastie, N. N. Jarjour, D. A. Meyers, J. Milosevic, W. C. Moore, J. R. Tedrow, J. B. Trudeau, H. P. Wong, W. Wu, N. Kaminski, S. E. Wenzel, B. D. Modena, BAL Cell Gene Expression in Severe Asthma Reveals Mechanisms of Severe Disease and Influences of Medications. Am J Respir Crit Care Med 200, 837-856 (2019).
66. S. Shah, E. M. King, A. Chandrasekhar, R. Newton, Roles for the mitogen-activated protein kinase (MAPK) phosphatase, DUSP1, in feedback control of inflammatory gene expression and repression by dexamethasone. J Biol Chem 289, 13667-13679 (2014).
67. J. Hoppstädter, A. J. Ammit, Role of Dual-Specificity Phosphatase 1 in Glucocorticoid-Driven Anti-inflammatory Responses. Frontiers in Immunology 10, (2019).
68. F. Sun, T.-T. Yue, C.-L. Yang, F.-X. Wang, J.-H. Luo, S.-J. Rong, M. Zhang, Y. Guo, F. Xiong, C.-Y. Wang, The MAPK dual specific phosphatase (DUSP) proteins: A versatile wrestler in T cell functionality. International Immunopharmacology 98, 107906 (2021).

69 D. Lu, L. Liu, X. Ji, Y. Gao, X. Chen, Y. Liu, Y. Liu, X. Zhao, Y. Li, Y. Li, Y. Jin, Y. Zhang, M. A. McNutt, Y. Yin, The phosphatase DUSP2 controls the activity of the transcription activator STAT3 and regulates TH17 differentiation. Nature Immunology 16, 1263-1273 (2015).
70. W. Y. Hsiao, Y. C. Lin, F. H. Liao, Y. C. Chan, C. Y. Huang, Dual-Specificity Phosphatase 4 Regulates STAT5 Protein Stability and Helper T Cell Polarization. PLoS One 10, e0145880 (2015).
71. S. Sakakibara, G. Espigol-Frigole, P. Gasperini, T. S. Uldrick, R. Yarchoan, G. Tosato, A20/TNFAIP3 inhibits NF-κB activation induced by the Kaposi's sarcoma-associated herpesvirus vFLIP oncoprotein. Oncogene 32, 1223-1232 (2013).
72. E. W. Harhaj, V. M. Dixit, Regulation of NF-κB by deubiquitinases. Immunol Rev 246, 107-124 (2012).
73. A. C. Anderson, N. Joller, V. K. Kuchroo, Lag-3, Tim-3, and TIGIT: Co-inhibitory Receptors with Specialized Functions in Immune Regulation. Immunity 44, 989-1004 (2016).
74. S. M. Lee, B. Gao, D. Fang, FoxP3 maintains Treg unresponsiveness by selectively inhibiting the promoter DNA-binding activity of AP-1. Blood 111, 3599-3606 (2008).
75. S.-i. Koizumi, D. Sasaki, T.-H. Hsieh, N. Taira, N. Arakaki, S. Yamasaki, K. Wang, S. Sarkar, H. Shirahata, M. Miyagi, H. Ishikawa, JunB regulates homeostasis and suppressive functions of effector regulatory T cells. Nature Communications 9, 5344 (2018).
76. H. Li, P. Su, T. K. Y. Lai, A. Jiang, J. Liu, D. Zhai, C. T. G. Campbell, F. H. F. Lee, W. Yong, S. Pasricha, S. Li, A. H. C. Wong, K. J. Ressler, F. Liu, The glucocorticoid receptor-FKBP51 complex contributes to fear conditioning and posttraumatic stress disorder. The Journal of Clinical Investigation 130, 877-889 (2020).
77. S. Sharma, A. T. Kho, D. Chhabra, W. Qiu, R. Gaedigk, C. A. Vyhlidal, J. S. Leeder, A. Barraza-Villarreal, S. J. London, F. Gilliland, B. A. Raby, S. T. Weiss, K. G. Tantisira, Glucocorticoid genes and the developmental origins of asthma susceptibility and treatment response. Am J Respir Cell Mol Biol 52, 543-553 (2015).
78. N. M. Galigniana, L. T. Ballmer, J. Toneatto, A. G. Erlejman, M. Lagadari, M. D. Galigniana, Regulation of the glucocorticoid response to stress-related disorders by the Hsp90-binding immunophilin FKBP51. Journal of Neurochemistry 122, 4-18 (2012).
79. B. J. Meckiff, C. Ramírez-Suástegui, V. Fajardo, S. J. Chee, A. Kusnadi, H. Simon, S. Eschweiler, A. Grifoni, E. Pelosi, D. Weiskopf, A. Sette, F. Ay, G. Seumois, C. H. Ottensmeier, P. Vijayanand, Imbalance of Regulatory and Cytotoxic SARS-COV-2-Reactive CD4(⁺) T Cells in COVID-19. Cell 183, 1340-1353.e1316 (2020).
80. V. S. Patil, A. Madrigal, B. J. Schmiedel, J. Clarke, P. O'Rourke, A. D. de Silva, E. Harris, B. Peters, G. Seumois, D. Weiskopf, A. Sette, P. Vijayanand, Precursors of human CD4(⁺) cytotoxic T lymphocytes identified by single-cell transcriptome analysis. Sci Immunol 3, (2018).
81. Y. Wang, Z. Chen, T. Wang, H. Guo, Y. Liu, N. Dang, S. Hu, L. Wu, C. Zhang, K. Ye, B. Shi, A novel CD4⁺ CTL subtype characterized by chemotaxis and inflammation is involved in the pathogenesis of Graves' orbitopathy. Cellular & Molecular Immunology 18, 735-745 (2021).
82. J. A. Juno, D. van Bockel, S. J. Kent, A. D. Kelleher, J. J. Zaunders, C. M. Munier, Cytotoxic CD4 T Cells-Friend or Foe during Viral Infection? Front Immunol 8, 19 (2017).
83. C. E. Hughes, R. J. B. Nibbs, A guide to chemokines and their receptors. FEBS J 285, 2944-2971 (2018).
84. T. Maehara, N. Kaneko, C. A. Perugino, H. Mattoo, J. Kers, H. Allard-Chamard, V. S. Mahajan, H. Liu, S. J. Murphy, M. Ghebremichael, D. Fox, A. S. Payne, R. Lafyatis, J. H. Stone, D. Khanna, S. Pillai, Cytotoxic CD4⁺ T lymphocytes may induce endothelial cell apoptosis in systemic sclerosis. J Clin Invest 130, 2451-2464 (2020).
85. H. Mattoo, V. S. Mahajan, T. Maehara, V. Deshpande, E. Della-Torre, Z. S. Wallace, M. Kulikova, J. M. Drijvers, J. Daccache, M. N. Carruthers, F. V. Castelino, J. R. Stone, J. H. Stone, S. Pillai, Clonal expansion of CD4(⁺) cytotoxic T lymphocytes in patients with IgG4-related disease. J Allergy Clin Immunol 138, 825-838 (2016).
86. L. K. Lundblad, J. Thompson-Figueroa, T. Leclair, M. J. Sullivan, M. E. Poynter, C. G. Irvin, J. H. Bates, Tumor necrosis factor-alpha overexpression in lung disease: a single cause behind a complex phenotype. Am J Respir Crit Care Med 171, 1363-1370 (2005)
87. S. Mukhopadhyay, J. R. Hoidal, T. K. Mukherjee, Role of TNFα in pulmonary pathophysiology. Respiratory Research 7, 125 (2006).
88. B. Becher, S. Tugues, M. Greter, GM-CSF: From Growth Factor to Central Mediator of Tissue Inflammation. Immunity 45, 963-973 (2016).
89. J. Zhao, C. M. Lloyd, A. Noble, Th17 responses in chronic allergic airway inflammation abrogate regulatory T-cell-mediated tolerance and contribute to airway remodeling. Mucosal Immunology 6, 335-346 (2013).
90. D. C. Newcomb, R. S. Peebles, Jr., Th17-mediated inflammation in asthma. Curr Opin Immunol 25, 755-760 (2013).
91. H. Nakajima, K. Hirose, Role of IL-23 and Th17 Cells in Airway Inflammation in Asthma. Immune Netw 10, 1-4 (2010).
92. H. S. Lee, D.-E. Park, J.-W. Lee, K. H. Sohn, S.-H. Cho, H.-W. Park, Role of interleukin-23 in the development of nonallergic eosinophilic inflammation in a murine model of asthma. Experimental & Molecular Medicine 52, 92-104 (2020).
93. L. Tortola, H. Pawelski, S. S. Sonar, F. Ampenberger, M. Kurrer, M. Kopf, IL-21 promotes allergic airway inflammation by driving apoptosis of FoxP3(⁺) regulatory T cells. J Allergy Clin Immunol 143, 2178-2189.e2175 (2019).
94. R. da Silva Antunes, A. K. Mehta, L. Madge, J. Tocker, M. Croft, TNFSF14 (LIGHT) Exhibits Inflammatory Activities in Lung Fibroblasts Complementary to IL-13 and TGF-β. Frontiers in Immunology 9, (2018).
95. H. N. Qiu, C. K. Wong, J. Dong, C. W. Lam, Z. Cai, Effect of tumor necrosis factor family member LIGHT (TNFSF14) on the activation of basophils and eosinophils interacting with bronchial epithelial cells. Mediators Inflamm 2014, 136463 (2014).
96. W. C. G. Fong, F. Borca, H. Phan, H. E. Moyses, P. Dennison, R. J. Kurukulaaratchy, H. M. Haitchi, Asthma did not increase in-hospital COVID-19-related mortality in a tertiary UK hospital. Clinical & Experimental Allergy 51, 939-941 (2021).
97. P. Haldar, I. D. Pavord, D. E. Shaw, M. A. Berry, M. Thomas, C. E. Brightling, A. J. Wardlaw, R. H. Green, Cluster analysis and clinical asthma phenotypes. Am J Respir Crit Care Med 178, 218-224 (2008).
98. V. Siroux, X. Basagaña, A. Boudier, I. Pin, J. Garcia-Aymerich, A. Vesin, R. Slama, D. Jarvis, J. M. Anto, F. Kauffmann, J. Sunyer, Identifying adult asthma phenotypes using a clustering approach. European Respiratory Journal 38, 310-317 (2011).
99. C. Newby, L. G. Heaney, A. Menzies-Gow, R. M. Niven, A. Mansur, C. Bucknall, R. Chaudhuri, J. Thompson, P. Burton, C. Brightling, Statistical cluster analysis of the British Thoracic Society Severe refractory Asthma Registry: clinical outcomes and phenotype stability. PLoS One 9, e102987 (2014).
100. P. Ilmarinen, L. E. Tuomisto, O. Niemelä, M. Tommola, J. Haanpää, H. Kankaanranta, Cluster Analysis on Longitudinal Data of Patients with Adult-Onset Asthma. J Allergy Clin Immunol Pract 5, 967-978.e963 (2017).
101. H. Cheroutre, M. M. Husain, CD4 CTL: living up to the challenge. Semin Immunol 25, 273-281 (2013).
102. D. Weiskopf, D. J. Bangs, J. Sidney, R. V. Kolla, A. D. De Silva, A. M. de Silva, S. Crotty, B. Peters, A. Sette, Dengue virus infection elicits highly polarized CX3CR1⁺ cytotoxic CD4⁺ T cells associated with protective immunity. Proc Natl Acad Sci USA 112, E4256-4263 (2015).
103. E. Derhovanessian, A. B. Maier, K. Hahnel, R. Beck, A. J. de Craen, E. P. Slagboom, R. G. Westendorp, G. Pawelec, Infection with cytomegalovirus but not herpes simplex virus induces the accumulation of late-differentiated CD4⁺ and CD8⁺ T-cells in humans. J Gen Virol 92, 2746-2756 (2011).
104. A. M. Intlekofer, N. Takemoto, E. J. Wherry, S. A. Longworth, J. T. Northrup, V. R. Palanivel, A. C. Mullen, C. R. Gasink, S. M. Kaech, J. D. Miller, L. Gapin, K. Ryan, A. P. Russ, T. Lindsten, J. S. Orange, A. W. Goldrath, R. Ahmed, S. L. Reiner, Effector and memory CD8+ T cell fate coupled by T-bet and comesodermin. Nature immunology 6, 1236-1244 (2005).
105. N. Takemoto, A. M. Intlekofer, J. T. Northrup, E. J. Wherry, S. L. Reiner, Cutting Edge: IL-12 inversely regulates T-bet and comesodermin expression during pathogen-induced CD8⁺ T cell differentiation. Journal of immunology 177, 7515-7519 (2006).
106. V. Appay, J. J. Zaunders, L. Papagno, J. Sutton, A. Jaramillo, A. Waters, P. Easterbrook, P. Grey, D. Smith, A. J. McMichael, D. A. Cooper, S. L. Rowland-Jones, A. D. Kelleher, Characterization of CD4(⁺) CTLs ex vivo. Journal of immunology 168, 5954-5958 (2002).
107. J. J. Zaunders, W. B. Dyer, B. Wang, M. L. Munier, M. Miranda-Saksena, R. Newton, J. Moore, C. R. Mackay, D. A. Cooper, N. K. Saksena, A. D. Kelleher, Identification of circulating antigen-specific CD4+T lymphocytes with a CCR5⁺, cytotoxic phenotype in an HIV-1 long-term nonprogressor and in CMV infection. Blood 103, 2238-2247 (2004).
108. P. J. Norris, H. F. Moffett, O. O. Yang, D. E. Kaufmann, M. J. Clark, M. M. Addo, E. S. Rosenberg, Beyond help: direct effector functions of human immunodeficiency virus type 1-specific CD4(⁺) T cells. Journal of virology 78, 8844-8851 (2004).
109. E. M. van Leeuwen, E. B. Remmerswaal, M. T. Vossen, A. T. Rowshani, P. M. Wertheim-van Dillen, R. A. van Lier, I. J. ten Berge, Emergence of a CD4+CD28− granzyme B⁺, cytomegalovirus-specific T cell subset after recovery of primary cytomegalovirus infection. Journal of immunology 173, 1834-1841 (2004).
110. N. Aslan, C. Yurdaydin, J. Wiegand, T. Greten, A. Ciner, M. F. Meyer, H. Heiken, B. Kuhlmann, T. Kaiser, H. Bozkaya, H. L. Tillmann, A. M. Bozdayi, M. P. Manns, H. Wedemeyer, Cytotoxic CD4 T cells in viral hepatitis. J Viral Hepat 13, 505-514 (2006).
111. T. M. Wilkinson, C. K. Li, C. S. Chui, A. K. Huang, M. Perkins, J. C. Liebner, R. Lambkin-Williams, A. Gilbert, J. Oxford, B. Nicholas, K. J. Staples, T. Dong, D. C. Douek, A. J. McMichael, X. N. Xu, Preexisting influenza-specific CD4⁺ T cells correlate with disease protection against influenza challenge in humans. Nat Med 18, 274-280 (2012).
112. C. Y. Fonseka, D. A. Rao, N. C. Teslovich, I. Korsunsky, S. K. Hannes, K. Slowikowski, M. F. Gurish, L. T. Donlin, J. A. Lederer, M. E. Weinblatt, E. M. Massarotti, J. S. Coblyn, S. M. Helfgott, D. J. Todd, V. P. Bykerk, E. W. Karlson, J. Ermann, Y. C. Lee, M. B. Brenner, S. Raychaudhuri, Mixed-effects association of single cells identifies an expanded effector CD4(⁺) T cell subset in rheumatoid arthritis. Science translational medicine 10, (2018).
113. D. Schafflick, C. A. Xu, M. Hartlehnert, M. Cole, A. Schulte-Mecklenbeck, T. Lautwein, J. Wolbert, M. Heming, S. G. Meuth, T. Kuhlmann, C. C. Gross, H. Wiendl, N. Yosef, G. Meyer Zu Horste, Integrated single cell analysis of blood and cerebrospinal fluid leukocytes in multiple sclerosis. Nature communications 11, 247 (2020).
114. S. R. Gilani, L. J. Vuga, K. O. Lindell, K. F. Gibson, J. Xue, N. Kaminski, V. G. Valentine, E. K. Lindsay, M. P. George, C. Steele, S. R. Duncan, CD28 down-regulation on circulating CD4 T-cells is associated with poor prognoses of patients with idiopathic pulmonary fibrosis. PLoS One 5, e8959 (2010).
115. D. Kim, G. Park, J. Huuhtanen, S. Lundgren, R. K. Khajuria, A. M. Hurtado, C. Munoz-Calleja, L. Cardenoso, V. Gomez-Garcia de Soria, T. H. Chen-Liang, S. Eldfors, P. Ellonen, S. Hannula, M. Kankainen, O. Bruck, A. Kreutzman, U. Salmenniemi, T. Lonnberg, A. Jerez, M. Itala-Remes, M. Myllymaki, M. A. I. Keranen, S. Mustjoki, Somatic mTOR mutation in clonally expanded T lymphocytes associated with chronic graft versus host disease. Nature communications 11, 2246 (2020).
116. R. Annoni, L. F. F. Silva, Y. Nussbaumer-Ochsner, A. van Schadewijk, T. Mauad, P. S. Hiemstra, K. F. Rabe, Increased expression of granzymes A and B in fatal asthma. European Respiratory Journal 45, 1485-1488 (2015).
117. Z. Zhou, H. He, K. Wang, X. Shi, Y. Wang, Y. Su, Y. Wang, D. Li, W. Liu, Y. Zhang, L. Shen, W. Han, L. Shen, J. Ding, F. Shao, Granzyme A from cytotoxic lymphocytes cleaves GSDMB to trigger pyroptosis in target cells. Science 368, (2020).
118. R. A. Panganiban, M. Sun, A. Dahlin, H. R. Park, M. Kan, B. E. Himes, J. A. Mitchel, C. Iribarren, E. Jorgenson, S. H. Randell, E. Israel, K. Tantisira, S. Shore, J. A. Park, S. T. Weiss, A. C. Wu, Q. Lu, A functional splice variant associated with decreased asthma risk abolishes the ability of gasdermin B to induce epithelial cell pyroptosis. J Allergy Clin Immunol 142, 1469-1478 e1462 (2018).
119. J. E. Wosen, D. Mukhopadhyay, C. Macaubas, E. D. Mellins, Epithelial MHC Class II Expression and Its Role in Antigen Presentation in the Gastrointestinal and Respiratory Tracts. Frontiers in immunology 9, 2144 (2018).
120. A. Agusti, L. M. Fabbri, D. Singh, J. Vestbo, B. Celli, F. M. Franssen, K. F. Rabe, A. Papi, Inhaled corticosteroids in COPD: Friend or foe? European Respiratory Journal, 1801219 (2018).
121. S. H. Arshad, J. W. Holloway, W. Karmaus, H. Zhang, S. Ewart, L. Mansfield, S. Matthews, C. Hodgekiss, G. Roberts, R. Kurukulaaratchy, Cohort Profile: The Isle Of Wight Whole Population Birth Cohort (IOWBC). Int J Epidemiol 47, 1043-1044i (2018).
122. S. Picelli, Å. K. Björklund, O. R. Faridani, S. Sagasser, G. Winberg, R. Sandberg, Smart-seq2 for sensitive full-length transcriptome profiling in single cells. Nature Methods 10, 1096-1098 (2013).
123. I. Engel, G. Seumois, L. Chavez, D. Samaniego-Castruita, B. White, A. Chawla, D. Mock, P. Vijayanand, M. Kronenberg, Innate-like functions of natural killer T cell subsets result from highly divergent gene programs. Nat Immunol 17, 728-739 (2016).
124. S. L. Rosales, S. Liang, I. Engel, B. J. Schmiedel, M. Kronenberg, P. Vijayanand, G. Seumois, A Sensitive and Integrated Approach to Profile Messenger RNA from Samples with Low Cell Numbers. Methods Mol Biol 1799, 275-302 (2018).
125. M. I. Love, W. Huber, S. Anders, Moderated estimation of fold change and dispersion for RNA-seq data with DESeq2. Genome Biol 15, 550 (2014).
126. 10× Genomics Cell Ranger 3.1.0.
127. H. M. Kang, M. Subramaniam, S. Targ, M. Nguyen, L. Maliskova, E. McCarthy, E. Wan, S. Wong, L. Byrnes, C. M. Lanata, R. E. Gate, S. Mostafavi, A. Marson, N. Zaitlen, L. A. Criswell, C. J. Ye, Multiplexed droplet single-cell RNA-sequencing using natural genetic variation. Nat Biotechnol 36, 89-94 (2018).
128. T. Stuart, A. Butler, P. Hoffman, C. Hafemeister, E. Papalexi, W. M. Mauck, 3rd, Y. Hao, M. Stoeckius, P. Smibert, R. Satija, Comprehensive Integration of Single-Cell Data. Cell 177, 1888-1902.e1821 (2019).
129. G. Korotkevich, V. Sukhov, N. Budin, B. Shpak, M. N. Artyomov, A. Sergushichev, Fast gene set enrichment analysis. bioRxiv, 060012 (2021).
130. G. Finak, A. McDavid, M. Yajima, J. Deng, V. Gersuk, A. K. Shalek, C. K. Slichter, H. W. Miller, M. J. McElrath, M. Prlic, P. S. Linsley, R. Gottardo, MAST: a flexible statistical framework for assessing transcriptional changes and characterizing heterogeneity in single-cell RNA sequencing data. Genome Biol 16, 278 (2015).
131. Busse, W. W., Banks-Schlegel, S. & Wenzel, S. E. Pathophysiology of severe asthma. J Allergy Clin Immunol 106, 1033-1042 (2000).
132. Gauthier, M., Ray, A. & Wenzel, S. E. Evolving Concepts of Asthma. Am J Respir Crit Care Med 192, 660-668 (2015).
133. Hammad, H. & Lambrecht, B. N. The basic immunology of asthma. Cell 184, 2521-2522 (2021).
134. D'Amato, G., et al. Asthma-related deaths. Multidiscip Respir Med 11, 37 (2016).
135. Hossny, E., Caraballo, L., Casale, T., El-Gamal, Y. & Rosenwasser, L. Severe asthma and quality of life. World Allergy Organ J 10, 28 (2017).
136. Bahadori, K., et al. Economic burden of asthma: a systematic review. BMC Pulm Med 9, 24 (2009).
137. Bousquet, J., et al. Uniform definition of asthma severity, control, and exacerbations: document presented for the World Health Organization Consultation on Severe Asthma. J Allergy Clin Immunol 126, 926-938 (2010).
138. Kuruvilla, M. E., Lee, F. E. & Lee, G. B. Understanding Asthma Phenotypes, Endotypes, and Mechanisms of Disease. Clin Rev Allergy Immunol 56, 219-233 (2019).
139. Azim, A., et al. New Perspectives on Difficult Asthma; Sex and Age of Asthma-Onset Based Phenotypes. J Allergy Clin Immunol Pract 8, 3396-3406 e3394 (2020).
140. Galli, S. J., Tsai, M. & Piliponsky, A. M. The development of allergic inflammation. 140. Nature 454, 445-454 (2008).
141. Wenzel, S. E. Emergence of Biomolecular Pathways to Define Novel Asthma Phenotypes. Type-2 Immunity and Beyond. Am J Respir Cell Mol Biol 55, 1-4 (2016).
142. Maggi, L., et al. The dual function of ILC2: From host protection to pathogenic players in type 2 asthma. Mol Aspects Med 80, 100981 (2021).
143. Cavagnero, K. J., et al. Unconventional ST2— and CD127-negative lung ILC2 populations are induced by the fungal allergen Alternaria alternata. J Allergy Clin Immunol 144, 1432-1435 e1439 (2019).
144. Holgate, S. T. Epithelium dysfunction in asthma. J Allergy Clin Immunol 120, 1233-1244; quiz 1245-1236 (2007).
145. Busse, W., et al. Omalizumab, anti-IgE recombinant humanized monoclonal antibody, for the treatment of severe allergic asthma. J Allergy Clin Immunol 108, 184-190 (2001).
146. Pavord, I. D., et al. Mepolizumab for severe eosinophilic asthma (DREAM): a multicentre, double-blind, placebo-controlled trial. Lancet 380, 651-659 (2012).
147. Menzella, F., Lusuardi, M., Galeone, C., Taddei, S. & Zucchi, L. Profile of anti-IL-5 mAb mepolizumab in the treatment of severe refractory asthma and hypereosinophilic diseases. J Asthma Allergy 8, 105-114 (2015).
148. FitzGerald, J. M., et al. Benralizumab, an anti-interleukin-5 receptor alpha monoclonal antibody, as add-on treatment for patients with severe, uncontrolled, eosinophilic asthma (CALIMA): a randomised, double-blind, placebo-controlled phase 3 trial. Lancet 388, 2128-2141 (2016).
149. Castro, M., et al. Dupilumab Efficacy and Safety in Moderate-to-Severe Uncontrolled Asthma. N Engl J Med 378, 2486-2496 (2018).
150. Woodruff, P. G., et al. T-helper type 2-driven inflammation defines major subphenotypes of asthma. Am J Respir Crit Care Med 180, 388-395 (2009).
151. Doberer, D., Trejo Bittar, H. E. & Wenzel, S. E. Should lung biopsies be performed in patients with severe asthma? European Respiratory Review 24, 525 (2015).
152. Azim, A., et al. Protocol for the Wessex AsThma CoHort of difficult asthma (WATCH): a pragmatic real-life longitudinal study of difficult asthma in the clinic. BMC Pulm Med 19, 99 (2019).
153. Larsson, K., et al. Bringing asthma care into the twenty-first century. npj Primary Care Respiratory Medicine 30, 25 (2020).
154. Global Initiative for Asthma. Global Strategy for Asthma Management and Prevention, 2021.
155. Patil, V. S., et al. Precursors of human CD4<sup>+</sup> cytotoxic T lymphocytes identified by single-cell transcriptome analysis. Science Immunology 3, eaan8664 (2018).
156. Morimoto, Y., et al. Amphiregulin-Producing Pathogenic Memory T Helper 2 Cells Instruct Eosinophils to Secrete Osteopontin and Facilitate Airway Fibrosis. Immunity 49, 134-150.e136 (2018).
157. Van Laethem, F., et al. Glucocorticoids attenuate T cell receptor signaling. J Exp Med 193, 803-814 (2001).
158. Woodruff, P. G., et al. Genome-wide profiling identifies epithelial cell genes associated with asthma and with treatment response to corticosteroids. Proc Natl Acad Sci USA 104, 15858-15863 (2007).
159. Kelly, M. M., et al. Corticosteroid-induced gene expression in allergen-challenged asthmatic subjects taking inhaled budesonide. Br J Pharmacol 165, 1737-1747 (2012).
160. Giordano, M., et al. The tumor necrosis factor alpha-induced protein 3 (TNFAIP3, A20) imposes a brake on antitumor activity of CD8 T cells. Proceedings of the National Academy of Sciences 111, 11115 (2014).
161. Xie, N., et al. NAD(+) metabolism: pathophysiologic mechanisms and therapeutic potential. Signal Transduct Target Ther 5, 227 (2020).
162. Clarke, J., et al. Single-cell transcriptomic analysis of tissue-resident memory T cells in human lung cancer. J Exp Med 216, 2128-2149 (2019).
163. Li, C., et al. The Transcription Factor Bhlhe40 Programs Mitochondrial Regulation of Resident CD8 (+) T Cell Fitness and Functionality. Immunity 51, 491-507 e497 (2019).
164. Seumois, G., et al. Single-cell transcriptomic analysis of allergen-specific T cells in allergy and asthma. Sci Immunol 5 (2020).
165. Hughes, C. E. & Nibbs, R. J. B. A guide to chemokines and their receptors. FEBS J 285, 2944-2971 (2018).
166. Koya, T., et al. RANTES (CCL5) regulates airway responsiveness after repeated allergen challenge. Am J Respir Cell Mol Biol 35, 147-154 (2006).
167. Rojas-Ramos, E., et al. Role of the chemokines RANTES, monocyte chemotactic proteins-3 and -4, and eotaxins-1 and -2 in childhood asthma. Eur Respir J 22, 310-316 (2003).
168. Zietkowski, Z., Tomasiak, M. M., Skiepko, R. & Bodzenta-Lukaszyk, A. RANTES in exhaled breath condensate of stable and unstable asthma patients. Respir Med 102, 1198-1202 (2008).
169. Balin, S. J., et al. Human antimicrobial cytotoxic T lymphocytes, defined by NK receptors and antimicrobial proteins, kill intracellular bacteria. Sci Immunol 3 (2018).
170. Smole, U., et al. Serum amyloid A is a soluble pattern recognition receptor that drives type 2 immunity. Nat Immunol 21, 756-765 (2020).
171. Itani, O. A., Liu, K. Z., Cornish, K. L., Campbell, J. R. & Thomas, C. P. Glucocorticoids stimulate human sgk1 gene expression by activation of a GRE in its 5′-flanking region. Am J Physiol Endocrinol Metab 283, E971-979 (2002).
172. Carlier, F. M., et al. Canonical WNT pathway is activated in the airway epithelium in chronic obstructive pulmonary disease. EBioMedicine 61, 103034 (2020).
173. Lam, A. P. & Gottardi, C. J. beta-catenin signaling: a novel mediator of fibrosis and potential therapeutic target. Curr Opin Rheumatol 23, 562-567 (2011).
174. Riccardo, P., et al. Expression of c-erbB receptors and ligands in the bronchial epithelium of asthmatic subjects. Journal of Allergy and Clinical Immunology 109, 75-81 (2002).
175. Chung, K. F., et al. International ERS/ATS guidelines on definition, evaluation and treatment of severe asthma. Eur Respir J 43, 343-373 (2014).
176. Vanderlugt, C. L. & Miller, S. D. Epitope spreading in immune-mediated diseases: implications for immunotherapy. Nat Rev Immunol 2, 85-95 (2002).
177. Miller, S. D., et al. Persistent infection with Theiler's virus leads to CNS autoimmunity via epitope spreading. Nat Med 3, 1133-1136 (1997).
178. McRae, B. L., Vanderlugt, C. L., Dal Canto, M. C. & Miller, S. D. Functional evidence for epitope spreading in the relapsing pathology of experimental autoimmune encephalomyelitis. J Exp Med 182, 75-85 (1995).
179. Faul, J. L., et al. Lung immunopathology in cases of sudden asthma death. Eur Respir J 10, 301-307 (1997).
180. O'Sullivan, S., et al. Activated, cytotoxic CD8(+) T lymphocytes contribute to the pathology of asthma death. Am J Respir Crit Care Med 164, 560-564 (2001).
181. Just, S., et al. A20 Curtails Primary but Augments Secondary CD8(+) T Cell Responses in Intracellular Bacterial Infection. Sci Rep 6, 39796 (2016).
182. Kästle, M., et al. FKBP51 modulates steroid sensitivity and NFκB signalling: A novel anti-inflammatory drug target. Eur J Immunol 48, 1904-1914 (2018).
183. Zannas, A. S., et al. Epigenetic upregulation of FKBP5 by aging and stress contributes to NF-κB-driven inflammation and cardiovascular risk. Proceedings of the National Academy of Sciences 116, 11370 (2019).
184. Tejwani, V., et al. Dexamethasone-Induced FKBP51 Expression in CD4(+) T-Lymphocytes Is Uniquely Associated With Worse Asthma Control in Obese Children With Asthma. Front Immunol 12, 744782 (2021).
185. Daniels, N. J., et al. Antigen-specific cytotoxic T lymphocytes target airway CD103+ and CD11b+ dendritic cells to suppress allergic inflammation. Mucosal Immunol 9, 229-239 (2016).
186. Enomoto, N., et al. Allergen-specific CTL require perforin expression to suppress allergic airway inflammation. J Immunol 188, 1734-1741 (2012).
187. Arshad, S. H., et al. Cohort Profile: The Isle Of Wight Whole Population Birth Cohort (IOWBC). Int J Epidemiol 47, 1043-1044i (2018).
188. Schmiedel, B. J., et al. Impact of Genetic Polymorphisms on Human Immune Cell Gene Expression. Cell 175, 1701-1715.e1716 (2018).
189. Picelli, S., et al. Smart-seq2 for sensitive full-length transcriptome profiling in single cells. Nature Methods 10, 1096-1098 (2013).
190. Engel, I., et al. Innate-like functions of natural killer T cell subsets result from highly divergent gene programs. Nat Immunol 17, 728-739 (2016).
191. Rosales, S. L., et al. A Sensitive and Integrated Approach to Profile Messenger RNA from Samples with Low Cell Numbers. Methods Mol Biol 1799, 275-302 (2018).
192. Love, M. I., Huber, W. & Anders, S. Moderated estimation of fold change and dispersion for RNA-seq data with DESeq2. Genome Biol 15, 550 (2014).
193. Seumois, G., et al. Single-cell transcriptomic analysis of allergen-specific T cells in allergy and asthma. Science Immunology 5, eaba6087 (2020).
194. 10× Genomics Cell Ranger 3.1.0.
195. Stuart, T., et al. Comprehensive Integration of Single-Cell Data. Cell 177, 1888-1902.e1821 (2019).
196. Finak, G., et al. MAST: a flexible statistical framework for assessing transcriptional changes and characterizing heterogeneity in single-cell RNA sequencing data. Genome Biol 16, 278 (2015).
197. Korotkevich, G., et al. Fast gene set enrichment analysis. bioRxiv, 060012 (2021).
198. Conway, J. R., Lex, A. & Gehlenborg, N. UpSetR: an R package for the visualization of intersecting sets and their properties. Bioinformatics 33, 2938-2940 (2017).

### ACCESSION CODES

Sequencing data are available from NCBI has Gene Expression Omnibus, SuperSeries accession number GSE181711 (Subseries: GSE181709 for bulk RNA-seq data and GSE181710 for single-cell RNA-seq data). The process to submit genotype data to the NCBI database of Genotypes and Phenotypes (dbGaP) has been initiated.

1. A method of treating asthma or an autoimmune or fibrotic disease in a subject comprising administering to the subject an effective amount of an agent to block the activity of a population of T-cells that exhibit higher than or lower than baseline expression of one or more genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1, thereby treating asthma or the autoimmune or fibrotic disease in the subject.

2. A method of treating asthma or an autoimmune or fibrotic disease in a subject comprising administering to the subject an effective amount of an agent that induces higher than or lower than baseline expression of one or more genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in T-cells, thereby treating asthma or the autoimmune or fibrotic disease in the subject.

3. A method of treating asthma or an autoimmune or fibrotic disease in a subject or sample comprising administering an effective amount of one or more of an agent that targets cells expressing a higher than or lower than baseline expression of one or more proteins encoded by genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 to the subject, thereby treating asthma or the autoimmune or fibrotic disease in the subject.

4. The method of claim 1, wherein the cells expressing higher than or lower than baseline expression are T-cells, optionally, CD4+ T-cells or CD8+ T-cells.

5. The method of claim 1, wherein the T-cells are tissue resident memory (TRM) cells.

6. The method of claim 1, wherein the agent is an antibody.

7. The method of claim 1, wherein the one or more genes comprises AMICA1.

8.-9. (canceled)

10. The method of claim 2, wherein the agent is an antibody, a small molecule, a protein, a peptide, a ligand mimetic or a nucleic acid.

11. The method of claim 1, further comprising administering to the subject an additional therapy for asthma or an autoimmune or fibrotic disease.

12. (canceled)

13. A method of diagnosing asthma or an autoimmune or fibrotic disease in a subject, comprising contacting a sample isolated from the subject with an agent that detects the presence of one or more genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in the sample isolated from the subject, wherein the presence of the one or more genes at higher or lower than baseline expression levels is a diagnostic indicator of asthma or the autoimmune or fibrotic disease or wherein the absence of the one or more genes at higher or lower than baseline expression levels is not diagnostic indicator of asthma or the autoimmune or fibrotic disease.

14. (canceled)

15. A method of diagnosing asthma or an autoimmune or fibrotic disease in a subject comprising contacting tissue-resident memory cells (TRMs) isolated from the subject or a sample isolated from the subject, with an antibody or agent that recognizes and binds one or more proteins encoded by a gene comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 to determine the frequency of TRMs expressing these proteins, wherein a high frequency of TRMs expressing these proteins is diagnostic of asthma or the autoimmune or fibrotic disease.

16. A method of determining the density of tissue-resident memory cells (TRMs) in a sample isolated from a subject comprising measuring expression of one or more genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in the sample, wherein higher or lower than baseline expression indicates higher density of TRMs in the sample thereof.

17. A method of determining prognosis of a subject having an autoimmune or fibrotic disease comprising measuring the density of tissue-resident memory cells (TRM) in a sample isolated from the subject, wherein a low density of TRM indicates a more positive prognosis or wherein a high density or TRM indicates a more negative prognosis optionally wherein the more negative prognosis comprises a decreased probability in the reduction of symptoms of the autoimmune or fibrotic disease and a lack of low density TRM indicates a more positive prognosis, optionally increased probability in the reduction of symptoms of the autoimmune or fibrotic or fibrotic disease.

18. A method of determining prognosis of a subject suffering from an autoimmune or fibrotic disease comprising contacting tissue-resident memory cells (TRMs) isolated from the subject with one or more of: an antibody or agent that recognizes and binds one or more proteins encoded by a gene comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1, wherein a low density of TRMs indicates a more positive prognosis or wherein a high density of TRMs indicates a more negative prognosis, optionally wherein the more negative prognosis comprises a decreased probability in the reduction of symptoms of the autoimmune or fibrotic disease, and wherein the more positive prognosis comprises an increased probability in the reduction of symptoms of the autoimmune or fibrotic disease.

19. A method of determining prognosis of a subject having an autoimmune or fibrotic disease comprising contacting tissue-resident memory cells (TRMs) isolated from the subject with an antibody or agent that recognizes and binds one or more proteins encoded by a genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs expressing these proteins, wherein a low density of TRMs indicates a more positive prognosis or wherein a high density of TRMs indicates a more negative prognosis, optionally wherein the more negative prognosis comprises a decreased probability in the reduction of symptoms of the autoimmune or fibrotic disease, and wherein the more positive prognosis comprises an increased probability in the reduction of symptoms of the autoimmune or fibrotic disease.

20. A method of determining prognosis of a subject having an autoimmune or fibrotic disease comprising contacting tissue-resident memory cells (TRMs) isolated from the subject with an antibody or agent that recognizes and binds CD103 to determine the frequency of CD103+ TRMs or an antibody that recognizes and binds a protein encoded by a genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs expressing the protein, wherein a low density of TRMs indicates a more positive prognosis or wherein a high density of TRMs indicates a more negative prognosis, optionally wherein the more negative prognosis comprises a decreased probability in the reduction of symptoms of the autoimmune or fibrotic disease, and wherein the more positive prognosis comprises an increased probability in the reduction of symptoms of the autoimmune or fibrotic disease.

21. A method of determining the responsiveness of a subject having asthma or an autoimmune or fibrotic disease to immunotherapy comprising contacting tissue-resident memory cells (TRMs) isolated from the subject with an antibody or agent that recognizes and binds one or more proteins encoded by a genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 to determine the frequency of TRMs in the subject, wherein a high frequency of TRMs indicates lack of responsiveness to immunotherapy.

22. A method of determining the responsiveness of a subject having asthma or an autoimmune or fibrotic disease to immunotherapy comprising contacting tissue-resident memory cells (TRMs) isolated from the subject with an antibody or agent that recognizes and binds one or more proteins encoded by a genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 and, to determine the frequency of TRMs expressing these proteins, wherein a low frequency of TRMs expressing these proteins indicates responsiveness to immunotherapy.

23. A method of determining prognosis of a subject having an autoimmune or fibrotic disease comprising measuring the density of CD103 or proteins encoded by one or more genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, or UMAP1 in a sample isolated from the subject, wherein a low density of TRM indicates a more positive prognosis or wherein a high density or TRM indicates a more negative prognosis optionally wherein the more negative prognosis comprises a decreased probability in the reduction of symptoms of the autoimmune or fibrotic disease and a lack of low density TRM indicates a more positive prognosis, optionally increased probability in the reduction of symptoms of the autoimmune or fibrotic disease.

24. A method of identifying a subject that will or is likely to respond to asthma therapy or an autoimmune or fibrotic disease therapy, comprising contacting a sample isolated from the subject with an agent that detects the presence of one or more genes comprising AMICA1, CD103, CD69, ITGAE, CD69, ITGA1, ZNF683, GZMA, GZMB, GZMH, CXCR4, CREM, PDE4B, DUSP1, DUSP2, MAF, DUSP4, TNFAIP3, HLA-DRB1, HOPX, HLA-DRA, FASLG, HLA-DPB1, HLA-DPA1, IFNG, LIGHT, CCL3, CCL4, CCL5, CLU, FKBP5, IL13, IL4, IL17, IL17A, IL21, TNF, CXADR, AREG, IL6ST, IL27RA, CXCR6, KLRC2, CLIC3, SLAMF6, KLRD1, KLRC4, and UMAP1 in the sample, wherein the presence of the one or more genes at higher or lower than baseline expression levels indicates that the subject is likely to respond to the asthma therapy or the autoimmune or fibrotic disease therapy.

25.-39. (canceled)
